SEQUENCE LISTING (74 sequences)

SEQ ID NO: 1 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 223 aa, Homo sapiens
MALPVTALLLPLALLLHAARPQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSAKPTTTPAPRPPTPAPTIASQPLSLRPEACRPAAGGAVHTRGLDFACDIYIWAPLAGTCGVLLLSLVITLY

SEQ ID NO: 3 - protein, 233 aa, Homo sapiens
MSTESMIRDVELAEEALPKKTGGPQGSRRCLFLSLFSFLIVAGATTLFCLLHFGVIGPQREEFPRDLSLISPLAQPVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 5 - protein, 41 aa, Homo sapiens
RSKRSRLLHSDYMNMTPRRPGPTRKHYQPYAPPRDFAAYRS

SEQ ID NO: 6 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 7 - DNA, 123 bp, Artificial Sequence
  Codon Optimized CD28 intracellular signaling domain
cggagcaagaggtcccgcctgctgcacagcgactatatgaacatgaccccacggagacccggccctacacggaaacattaccagccctatgctccaccccgggacttcgcagcttacagaagt

SEQ ID NO: 8 - protein, 22 aa, Homo sapiens
ERVQPLEENVGNAARPRFERNK

SEQ ID NO: 9 - protein, 17 aa, Homo sapiens
LEENVGNAARPRFERNK

SEQ ID NO: 10 - protein, 8 aa, Homo sapiens
RPRFERNK

SEQ ID NO: 11 - DNA, 66 bp, Homo sapiens
gaaagggtccaacccctggaagagaatgtgggaaatgcagccaggccaagattcgagaggaacaag

SEQ ID NO: 12 - DNA, 66 bp, Artificial Sequence
  Codon optimized OX40L intracellular signaling domain
gaaagagtgcagcccctggaagagaatgtcgggaatgccgctcgcccaagatttgaaaggaacaaa

SEQ ID NO: 13 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 15 - DNA, 336 bp, Artificial Sequence
  Codon optimized CD3zeta signaling domain
cgagtgaagttcagcaggtccgccgacgctcctgcataccagcagggacagaaccagctgtataacgagctgaatctgggccggagagaggaatacgacgtgctggacaaaaggcggggccgggaccccgaaatgggagggaagccacgacggaaaaacccccaggagggcctgtacaatgagctgcaaaaggacaaaatggccgaggcttattctgaaatcgggatgaagggagagagaaggcgcggaaaaggccacgatggcctgtaccaggggctgagcaccgctacaaaggacacctatgatgcactgcacatgcaggccctgccccctcgg

SEQ ID NO: 16 - protein, 9 aa, Artificial Sequence
  2A cleavage motif
  misc_feature (5)..(5): Xaa can be any naturally occurring amino acid
GDVEXNPGP

SEQ ID NO: 17 - protein, 21 aa, Artificial Sequence
  T2A cleavage site
GSGEGRGSLLTCGDVEENPGP

SEQ ID NO: 18 - protein, 63 aa, Artificial Sequence
  T2A cleavage site
GGCTCAGGTGAGGGGCGCGGGAGCCTGCTGACTTGTGGGGATGTAGAGGAAAATCCTGGTCCT

SEQ ID NO: 19 - protein, 22 aa, Artificial Sequence
  P2A cleavage site
GSGATNFSLLKQAGDVEENPGP

SEQ ID NO: 20 - protein, 23 aa, Artificial Sequence
  E2A cleavage site
GSGQCTNYALLKLAGDVESNPGP

SEQ ID NO: 21 - protein, 25 aa, Artificial Sequence
  F2A cleavage site
GSGVKQTLNFDLLKLAGDVESNPGP

SEQ ID NO: 22 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 23 - DNA, 489 bp, Homo sapiens
atgagaatcagcaaaccacacctccggagcatatcaatccagtgttacttgtgccttcttttgaactcccatttcctcaccgaggcaggcattcatgtgttcatattggggtgctttagtgctgggcttccgaaaacggaagctaactgggtaaacgtcatcagtgaccttaaaaaaattgaggatcttatccaatcaatgcacatcgacgcgactctctacacagaatctgacgtacacccgtcatgcaaagtcacggcaatgaagtgttttcttctcgagctccaagtaatttccctggagtctggcgatgcctccatccacgatacggttgaaaatctgattatattggccaacaatagcctcagttctaacggtaacgtgactgaaagtggctgcaaagagtgcgaagagctcgaagaaaagaatatcaaggagttcctccaatcatttgttcacattgtgcaaatgtttatcaacacctcttga

SEQ ID NO: 24 - DNA, 489 bp, Homo sapiens
atgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 25 - protein, 175 aa, Artificial Sequence
  CD28/OX40L/CD3zeta
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 358 aa, Artificial Sequence
  CD28/OX40L/CD3zeta/T2A/IL1-5
RSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 27 - protein, 21 aa, Homo sapiens
MALPVTALLLPLALLLHAARP

SEQ ID NO: 28 - DNA, 63 bp, Homo sapiens
atggccttaccagtgaccgccttgctcctgccgctggccttgctgctccacgccgccaggccg

SEQ ID NO: 29 - DNA, 63 bp, Artificial Sequence
  Codon Optimized CD8alpha signal sequence
atggcacttcctgttacagccctcctgctcccactggctttgctgctgcatgctgcacgaccg

SEQ ID NO: 30 - protein, 242 aa, Artificial Sequence
  anti-CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - DNA, 726 bp, Artificial Sequence
  anti-CD19 scFv
gacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagc

SEQ ID NO: 32 - protein, 12 aa, Artificial Sequence
  IgG1 hinge
EPKSCDKTHTCP

SEQ ID NO: 33 - DNA, 36 bp, Artificial Sequence
  IgG1 hinge
gagccaaagtcttgcgacaagactcatacctgcccg

SEQ ID NO: 34 - protein, 219 aa, Artificial Sequence
  Spacer (IgG1, CH2 (mu) - CH3
PCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - DNA, 336 bp, Artificial Sequence
  Spacer (IgG1, CH2 (mu) - CH3
ccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaag

SEQ ID NO: 36 - protein, 220 aa, Artificial Sequence
  Spacer (IgG1, CH2 (mu) - CH3
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 657 bp, Artificial Sequence
  Spacer (IgG1, CH2 (mu) - CH3
ccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaag

SEQ ID NO: 38 - protein, 27 aa, Artificial Sequence
  CD28 transmembrane domain
FWVLVVVGGVLACYSLLVTVAFIIFWV

SEQ ID NO: 39 - DNA, 81 bp, Artificial Sequence
  CD28 transmembrane domain
ttctgggtactggtggttgtgggcggcgttctggcttgctattcactcctcgttacggttgcatttataatattttgggta

SEQ ID NO: 40 - protein, 24 aa, Artificial Sequence
  CD8 transmembrane domain
IYIWAPLAGTCGVLLLSLVITLYC

SEQ ID NO: 41 - DNA, 72 bp, Artificial Sequence
  CD8 transmembrane domain
atctacatttgggcccctctggctggaacatgtggcgtgctgctgctgtccctggtcattactctgtattgt

SEQ ID NO: 42 - protein, 697 aa, Artificial Sequence
  CD19 CAR-WT-28 (with signal)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 2091 bp, Artificial Sequence
  CD19 CAR-WT-28
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccagagttgctgggtggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatataatagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagttctgggtactggtggttgtgggcggcgttctggcttgctattcactcctcgttacggttgcatttataatattttgggtacgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccaga

SEQ ID NO: 44 - protein, 880 aa, Artificial Sequence
  CD19 CAR-WT-28 with T2A and IL-15
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 45 - DNA, 2643 bp, Artificial Sequence
  CD19 CAR-WT-28 with T2A and IL-15
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccagagttgctgggtggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatataatagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagttctgggtactggtggttgtgggcggcgttctggcttgctattcactcctcgttacggttgcatttataatattttgggtacgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccagaggaagtggggaaggccgaggttcattgctcacctgtggcgatgtggaagaaaatccaggtccgatgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 46 - protein, 696 aa, Artificial Sequence
  CD19 CAR-MU-28 (with signal)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 2088 bp, Artificial Sequence
  CD19 CAR-MU-28
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagttctgggtactggtggttgtgggcggcgttctggcttgctattcactcctcgttacggttgcatttataatattttgggtacgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccaga

SEQ ID NO: 48 - protein, 879 aa, Artificial Sequence
  CD19 CAR-MU-28 with T2A and IL-15
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 2640 bp, Artificial Sequence
  CD19 CAR-MU-28 with T2A and IL-15
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagttctgggtactggtggttgtgggcggcgttctggcttgctattcactcctcgttacggttgcatttataatattttgggtacgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccagaggaagtggggaaggccgaggttcattgctcacctgtggcgatgtggaagaaaatccaggtccgatgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 50 - protein, 694 aa, Artificial Sequence
  CD19 CAR-WT-8 (with signal)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 51 - DNA, 2082 bp, Artificial Sequence
  CD19 CAR-WT-8
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccagagttgctgggtggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatataatagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagatctacatttgggcccctctggctggaacatgtggcgtgctgctgctgtccctggtcattactctgtattgtcgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccaga

SEQ ID NO: 52 - protein, 877 aa, Artificial Sequence
  CD19 CAR-WT-8 with T2A and IL-15 (with signal)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - DNA, 2634 bp, Artificial Sequence
  CD19 CAR-WT-8 with T2A and IL-15
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccagagttgctgggtggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatataatagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagatctacatttgggcccctctggctggaacatgtggcgtgctgctgctgtccctggtcattactctgtattgtcgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccagaggaagtggggaaggccgaggttcattgctcacctgtggcgatgtggaagaaaatccaggtccgatgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 54 - protein, 693 aa, Artificial Sequence
  CD19 CAR-MU-8 (with signal)
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - DNA, 2079 bp, Artificial Sequence
  CD19 CAR-MU-8
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagatctacatttgggcccctctggctggaacatgtggcgtgctgctgctgtccctggtcattactctgtattgtcgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccaga

SEQ ID NO: 56 - protein, 876 aa, Artificial Sequence
  CD19 CAR-MU-8 with T2A and IL-15
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 57 - DNA, 2631 bp, Artificial Sequence
  CD19 CAR-MU-8 with T2A and IL-15
atggcacttcctgttacagccctcctgctcccactggctttgctgctgcatgctgcacgaccggacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagatctacatttgggcccctctggctggaacatgtggcgtgctgctgctgtccctggtcattactctgtattgtcgctcaaagcgctcaagattgctccattctgattacatgaacatgacccctcgccggccgggtcccactcgcaaacactaccaaccttatgcccctccgcgggatttcgcggcctacaggtcagagagagtacaacctcttgaggagaatgtcggcaacgctgccaggccgcggtttgagcgaaataagcgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccagaggaagtggggaaggccgaggttcattgctcacctgtggcgatgtggaagaaaatccaggtccgatgcgcataagtaagcctcatctgcggtccatttctatacaatgttatctgtgcttgcttttgaactcccactttcttacggaagcaggcattcatgtgttcattctgggttgtttttctgccgggctgcccaaaaccgaggccaactgggtcaacgtgatcagcgacctcaagaagatcgaggatttgattcaaagtatgcatatagacgccacactctatactgagtccgacgttcacccgagttgtaaagttacggctatgaagtgctttttgttggaactccaggtgatttcccttgaatccggcgatgcgagcatccacgatacggtagagaatcttattattctggcgaataattctctgtcttcaaatgggaatgtaactgagagcggttgtaaagaatgcgaagaacttgaagaaaagaatatcaaggaatttcttcagagtttcgtgcatattgttcaaatgttcatcaacacatcctga

SEQ ID NO: 58 - protein, 10 aa, Artificial Sequence
  anti-CD19 scFv  CDRL1
RASQDISKYL

SEQ ID NO: 59 - protein, 7 aa, Artificial Sequence
  anti-CD19 scFv  CDRL2
HTSRLHS

SEQ ID NO: 60 - protein, 9 aa, Artificial Sequence
  anti-CD19 scFv  CDRL3
QQGNTLPYT

SEQ ID NO: 61 - protein, 5 aa, Artificial Sequence
  anti-CD19 scFv  CDRH1
DYGVS

SEQ ID NO: 62 - protein, 16 aa, Artificial Sequence
  anti-CD19 scFv  CDRH2
VIWGSETTYYNSALKS

SEQ ID NO: 63 - protein, 12 aa, Artificial Sequence
  anti-CD19 scFv  CDRH3
HYYYGGSYAMDY

SEQ ID NO: 64 - protein, 107 aa, Artificial Sequence
  anti-CD19 scFv  VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 65 - protein, 120 aa, Artificial Sequence
  anti-CD19 scFv  VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 66 - protein, 556 aa, Homo sapiens [sequence omitted]

SEQ ID NO: 67 - protein, 15 aa, Artificial Sequence
  spacer
GGGGSGGGGSGGGGS

SEQ ID NO: 68 - protein, 609 aa, Homo sapiens [sequence omitted]

SEQ ID NO: 69 - protein, 676 aa, Artificial Sequence
  CD19 CAR-WT-28
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 70 - protein, 675 aa, Artificial Sequence
  CD19 CAR-MU-28
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 673 aa, Artificial Sequence
  CD19 CAR-WT-8
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 672 aa, Artificial Sequence
  CD19 CAR-MU-8
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCRSKRSRLLHSDYMNMTPRRPGPTRKHYQPYAPPRDFAAYRSERVQPLEENVGNAARPRFERNKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 653 aa, Artificial Sequence
  Anti-CD19 CAR (CD28-CD3z-IL15)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSEPKSCDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - DNA, 1959 bp, Artificial Sequence
  Anti-CD19 CAR (CD28-CD3z-IL15)
gacattcaaatgacacaaactacgagctccctctccgcctccctcggtgacagggtgaccatctcatgtcgggctagtcaggacataagcaaatacttgaactggtaccagcaaaagccagacgggaccgtgaagctgctcatctatcacactagccgccttcactctggggtcccctctcgctttagcggttccggctctggtacagactattcccttactatttcaaatcttgagcaggaagatatcgctacatatttttgtcaacaggggaatactcttccgtatacattcgggggcggaacaaaacttgaaattacaggcggcggtggttcaggaggtggtgggagcggtggggggggatcagaagttaagctgcaagaatcagggcccggactggtagcgcccagtcaaagtctttctgttacttgtacagtatccggggtgtcccttccagattacggggtatcttggattaggcaacctcctcggaaaggattggagtggttgggtgttatctggggcagtgaaaccacttactataatagtgctcttaaaagtagactcaccataataaaggataatagcaagagccaggtattcctgaaaatgaacagcttgcaaacggatgataccgctatctactactgcgccaaacactattattatggcggtagttacgctatggattattggggccaaggaacgtccgtcaccgttagtagcgagccaaagtcttgcgacaagactcatacctgcccgccttgtcccgcaccacctgtggcaggtcccagcgtgtttctgtttccgcccaagcccaaggacacactcatgatctctaggacacccgaggttacatgcgtcgtggttgatgtttctcatgaagaccccgaagtgaagtttaactggtatgtcgacggagttgaggtccataatgctaaaacaaaaccacgcgaggaacaatatcaaagcacctatcgggtcgttagtgtccttactgtacttcaccaggactggctcaacggaaaggaatataagtgcaaagtcagcaataaagccttgccagcgcccatcgaaaaaactatcagcaaggccaagggccaacccagagaaccccaagtgtacacactccctcctagccgggatgaattgaccaaaaatcaagtctctctcacttgtctggtaaagggattctatccaagtgatattgctgtcgaatgggagagcaacgggcaaccggagaacaattacaaaacgacacctcccgtccttgatagtgacgggtccttcttcctgtattcaaagcttacagtcgacaaaagtcgctggcagcaggggaatgtctttagctgtagtgtcatgcacgaagctttgcataaccactacacgcaaaagtctcttagcctgtccccaggaaagttctgggtactggtggttgtgggcggcgttctggcttgctattcactcctcgttacggttgcatttataatattttgggtacgctcaaagcgctcaagattgctccattctgattacatgaacatgacccctcgccggccgggtcccactcgcaaacactaccaaccttatgcccctccgcgggatttcgcggcctacaggtcacgagtcaagttttctcgaagtgccgacgctcctgcctatcagcaaggccagaatcaactgtataacgagcttaacttgggccggagggaggagtacgatgtgctggataagcgcaggggacgggacccggaaatgggcggaaagcccaggcggaaaaacccacaggaaggactctacaatgaactccaaaaggacaaaatggcagaggcctattcagagattgggatgaagggagaaaggaggcgaggaaaagggcacgacggcttgtatcagggcttgtctacggccactaaagacacctatgatgccctgcatatgcaggcacttccccccaga

(19) United States
(12) Patent Application Publication
(10) Pub. No.: US 2024/0366663 A1
(43) Pub. Date: Nov. 7, 2024

(54) CHIMERIC ANTIGEN RECEPTOR COMPRISING AN ANTI-CD19 ANTIBODY OR ANTIGEN-BINDING FRAGMENT THEREOF AND NATURAL KILLER CELLS COMPRISING THE SAME

(71) Applicants: Artiva Biotherapeutics, Inc., San Diego, CA (US); GC Cell Corporation, Yongin-si Gyeonggi-do (KR)

(72) Inventors: Yusun KIM, Gyeonggi-do (KR); Sungyoo CHO, Gyeonggi-do (KR); Yu Kyeong HWANG, Gyeonggi-do (KR); Peter FLYNN, Cardiff by the Sea, CA (US); Jason B. LITTEN, Poway, CA (US); Thomas James FARRELL, La Jolla, CA (US); John Kin Chuan LIM, San Diego, CA (US)

(21) Appl. No.: 18/285,634

(22) PCT Filed: Apr. 6, 2022
(86) PCT No.: PCT/US2022/023654

§ 371 Date: Oct. 4, 2023

Related U.S. Application Data
(60) Provisional application No. 63/172,416, filed on Apr. 8, 2021.

(51) Int. Cl. A61K 35/17; A61K 39/00; A61P 35/00; C07K 16/28

(52) U.S. Cl. CPC A61K 35/17; A61K 39/4613; A61K 39/4631; A61K 39/464412; A61P 35/00; C07K 16/2803; A61K 2239/13; A61K 2239/21; A61K 2239/22; C07K 2317/565

(57) ABSTRACT

Provided herein, among other things, are anti-CD 19 chimeric antigen receptor (CAR)s and natural killer cells expressing the same.

### CLAIM OF PRIORITY

This application claims the benefit of U.S. Provisional Application Ser. No. 63/172,416, filed on Apr. 8, 2021. The entire contents of the foregoing are incorporated herein by reference.

### BACKGROUND

Targeted therapies, including the use of adoptive cell therapies such as chimeric antigen receptor T cells (CAR Ts), have revolutionized cancer treatment. These cell therapies may be autologous (CAR T cells manufactured using a patient's own T cells) or allogeneic (CAR T cells manufactured using T cells from healthy donors). Autologous CAR-T therapies targeting CD19-specific hematologic malignancies have resulted in significant remission responses. Currently, there are multiple autologous CAR T therapies approved to treat CD19-specific hematologic cancers.

Whilst transformative in their ability to treat targeted hematologic cancers, challenging obstacles have arisen in the clinic since 2017, when the first CAR-T therapies targeting CD19 B-cell malignancies were approved by the United States Food and Drug Administration, with the use of autologous CAR-T cell products. CAR T cell manufacturing is a resource-intensive process that can result in failure to produce a viable autologous cell therapy for some patients. The average manufacturing time of 3 weeks that is needed for autologous CAR T cell products may be too long for critically ill patients. Finally, due to the complex nature of the manufacture and delivery of CAR-T cell product, which require close monitoring at top-tier cancer and medical centers, access to this treatment option may be out of reach, both financially and geographically, for most patients. Importantly, even for those patients who have access to this innovative treatment, CAR-T cell products confer a risk of serious and potentially deadly adverse effects. These adverse effects include cytokine release syndrome (CRS) and neurotoxicity, which can be difficult to manage or control.

Allogeneic CAR-T cell therapies, which utilize cells from healthy donors, may overcome some of the manufacturing and logistical challenges of autologous CAR-T cell therapies. However, these “off-the-shelf” CAR T cell therapies also have issues that include a potentially higher risk of graft-versus-host disease (GVHD) and ineffectiveness due to rapid clearance by the patient's immune system.

Natural killer (NK) cells are cytolytic cells of the innate immune system with an intrinsic ability to lyse tumor cells and virus-infected cells. Prior antigen exposure is not required for NK cells to identify and lyse tumor cells. Natural killer cells have the inherent ability to bridge between innate immunity and engender a multi-clonal adaptive immune response resulting in long-term anticancer immune memory. Receptor engagement by NK cells drives effector function through degranulation of lytic granules, activation of programmed cell death receptors on target cells, and secretion of immune modulatory cytokines. The effector function of NK cells is governed through the balance of activating and inhibitory receptor signaling. Classically, NK cells are defined as CD56⁺ and CD3⁻ cells that are subdivided in to CD56^{bright}CD16⁻, cytokine secreting cells and CD56^{dim}CD16⁺ cytolytic cells (Carson 1997, Cooper 2001).

Natural killer cells also engage tumor cells through antibody dependent cellular cytotoxicity (ADCC), a key component of the innate immune system. Antibody-coated target cells are killed by cells with Fc receptors that recognize the constant region of the bound antibody. Engagement of CD16 (FCγRIII) with antibody-opsonized tumor cells is sufficient to elicit cytotoxicity and cytokine release response by resting NK cells. Activated NK cells secrete cytokines and chemokines, such as interferon gamma (IFNγ); tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system. ADCC is recognized as a potent mechanism of NK cell action, particularly in combination with antibodies belonging to immunoglobulin G1 (IgG1) and IG3 subclasses. To initiate ADCC, NK cells engage with antibodies via the CD16 receptor.

Similar to T cells, allogeneic NK cells engineered to express CARs with anti-tumor activity may provide an important treatment option for cancer patients. NK cells do not suffer from some of the shortcomings of allogeneic CAR-T cells, which often retain expression of endogenous T cell receptors in addition to engineered chimeric antigen receptors. As a result, allogeneic CAR-NK cell treatments can be administered safely to patients without many of the risks associated with allogeneic T cell therapies, including GVHD. However, CAR-NK cells face many of the same challenges as other allogeneic cell therapies, including product sourcing, scalability, persistence, and dose-to-dose variability.

CD19 is a human antigen belonging to the immunoglobulin superfamily. This type I transmembrane protein is present on both healthy and malignant B cells. Thus, there is an unmet need for an allogeneic anti-CD19 CAR NK cell therapy that provides efficacy against CD19-specific hematologic cancers but, unlike autologous CAR-T cell therapies, offers a better safety profile for patients, can be manufactured in bulk, cryopreserved, shipped, stored, and administered in community hospitals rather than elite academic centers.

The present invention addresses these and other deficiencies in the art.

### SUMMARY

NK cells are immune cells that can engage tumor cells through a complex array of receptors on their cell surface, as well as through antibody-dependent cellular cytotoxicity (ADCC). NK cells may have an advantage over other immune cells, such as the T cells used in CAR-T cell therapy and other cell therapies. Autologous CAR-T cells must be engineered from a patient's own cells. Such engineering can take time, during which period the patient's disease may progress significantly. Such patients may require a bridging therapy to sustain them until their autologous CAR-T cells are ready. Not all patients qualify for autologous CAR-T therapy. For example, some patients may be too sick or may not have sufficient numbers of T cells suitable for engineering purposes. Not all manufacturing runs of autologous CAR-T cells result in sufficient cell numbers or sufficiently active cells to be therapeutically effective. When such manufacturing runs are successful, patients typically only receive a single dose of autologous CAR-T treatment. Because the risk of acute side effects like ICANS and CRS are greatest immediately after administering CAR-T cells, repeat dosing is potentially too risky if the patient will only see marginal benefit from a second, third, or further dose. Additionally, because autologous CAR-T treatments must be unique for each patient, the costs of such treatments can make them unaffordable for many patients who would otherwise benefit from them.

In an exemplary advantage, NK cells can be used as allogeneic therapies, meaning that NK cells from one donor can be safely used in one or many patients without the requirement for HLA matching, gene editing, or other genetic manipulations. As a result, allogeneic CAR-NK cells can be manufactured in bulk, cryopreserved, shipped throughout the world, and administered on demand at the point of care. Thus, the allogeneic cell therapies can be administered to a patient immediately, without the need to wait for the patient's own cells to be engineered and administered and without the need for a bridging therapy. Because the allogeneic therapies described herein can be manufactured in bulk using campaign-manufacturing methods, the costs associated with manufacturing and delivering the allogeneic therapies described herein has the promise to be significantly lower than those of autologous CAR-T therapies. Campaign manufacturing also reduces variability between batches and allows a patient to receive multiple doses of CAR-NK cells made from a single batch derived from a single donor where preferable.

The ability to offer repeat dosing may allow patients to experience or maintain a deeper or prolonged response from the therapy. For example, patients can receive response-based dosing, during which the patient continues to receive doses of CAR-NK cell therapy for as long as the patient derives a benefit. The number of doses and the number of cells administered in each dose can also be tailored to the individual patient. In such cases, the patient is not limited by the number of cells he or she can provide during the cell harvests associated with autologous CAR-T therapy. Thus, the CAR-NK cell therapies described herein can be tailored to each patient based on that patient's own response. In some cases, the therapy can also be reinitiated if the patient relapses.

Allogeneic NK cells may provide an important treatment option for cancer patients. In one exemplary advantage, NK cells have been well tolerated without evidence of graft-versus-host disease, neurotoxicity or cytokine release syndrome associated with other cell-based therapies. In another exemplary advantage, NK cells do not require prior antigen exposure to identify and lyse tumor cells. In another exemplary advantage, NK cells have the inherent ability to bridge between innate immunity and engender a multi-clonal adaptive immune response resulting in long-term anticancer immune memory. All of these features contribute to the potential for NK cell efficacy as cancer treatment options.

For example, NK cells can recruit and activate other components of the immune system. Activated NK cells secrete cytokines and chemokines, such as interferon gamma (IFNγ); tumor necrosis factor alpha (TNFα); and macrophage inflammatory protein 1 (MIP1) that signal and recruit T cells to tumors. Through direct killing of tumor cells, NK cells also expose tumor antigens for recognition by the adaptive immune system.

Additionally, umbilical cord blood units with preferred characteristics for enhanced clinical activity (e.g., high-affinity CD16 and Killer cell Immunoglobulin-like Receptor (KIR) B-haplotype) can be selected by utilizing a diverse umbilical cord blood bank as a source for NK cells.

The administration of the NK cells engineered to express CARs described herein, can enhance the targeting and activity of the engineered NK cells in patients. Engineered NK cells, e.g., the CAR-NK cells described herein, have an advantage over autologous cell therapies, e.g., T cells used in CAR-T cell therapy, because the NK cells can be used as allogeneic therapies. Thus, NK cells from one donor, such as a single cord blood donor or NK cells derived from a single unit of cord blood, can be safely used in one or many patients.

The CAR-NK cells described herein, e.g., AB-202, are an off-the-shelf allogenic anti-CD19 CAR-NK cell product manufactured from cord blood, useful in the treatment of disease, e.g., cancer, e.g., CD-19+ cancers, e.g., B-cell malignancies. The CAR-NK cells described herein, e.g., AB-202, are not plagued by the unwanted side effects or manufacturing challenges of autologous T cell therapies.

Patients treated with anti-CD19 CAR-T cell therapies experience relapse, which, in up to 75% of patients, is due to the loss of CD19 expression. The CAR-NK cells described herein can retain CD16 expression, including expression of the 158 V/V variant of CD16. Thus, in some cases, the CAR-NK cells can be used in combination with traditional antibody therapy. In one example, the antibody therapy can comprise rituximab or obinutuzumab. Such antibodies can elicit an ADCC response from NK cells by binding to CD16 expressed on the NK cell surface. Thus, in some cases, the method of treatment can include a dual targeting approach comprising combining the use of the CAR-NK cells targeting CD19 described herein with an anti-CD20 antibody therapy.

Thus, provided herein, amongst other things, are anti-CD19 chimeric antigen receptor (CAR)s and natural killer cells expressing the same.

Provided herein are anti-CD19 chimeric antigen receptor(s) (CARs) comprising: an extracellular antigen binding domain comprising an anti-CD19 antibody or antigen binding fragment thereof; and an intracellular signaling region comprising an OX40L intracellular signaling domain.

In some embodiments, the OX40L intracellular signaling domain comprises an amino acid sequence set forth in SEQ ID NO: 8, SEQ ID NO: 9, or SEQ ID NO: 10. In some embodiments, the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10.

In some embodiments, the anti-CD19 antibody or antigen binding fragment thereof comprises a light chain complementarity determining region 1 (CDRL1) comprising SEQ ID NO: 58, a light chain complementarity determining region 2 (CDRL2) comprising SEQ ID NO: 59; a light chain complementarity determining region 3 (CDRL3) comprising SEQ ID NO: 60, a heavy chain complementarity determining region 1 (CDRH1 comprising SEQ ID NO: 61; a heavy chain complementarity determining region 2 (CDRH2) comprising SEQ ID NO: 62; and a heavy chain complementarity determining region 3 (CDRH3) comprising SEQ ID NO: 63.

In some embodiments, the anti-CD19 antibody or antigen binding fragment thereof comprises a light chain variable (V_{L}) region comprising SEQ ID NO: 64 and a heavy chain variable (V_{H}) region comprising SEQ ID NO: 65. In some embodiments, the anti-CD19 antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 64 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 65.

In some embodiments, the anti-CD19 antibody or antigen binding fragment thereof is an antigen binding fragment. In some embodiments, the antigen binding fragment comprises a single chain Fv (scFv). In some embodiments, the V_{L} region is amino-terminal to the V_{H} region. In some embodiments, the V_{L} region is carboxy-terminal to the V_{H} region. In some embodiments, the V_{L} region is joined to the V_{H} region via a flexible linker. In some embodiments, the flexible linker comprises the amino acid sequence set forth in SEQ ID NO: 67. In some embodiments, the scFv comprises the amino acid sequence set forth in SEQ ID NO: 30. In some embodiments, the scFv comprises an amino acid sequence having or having at least 85%, 86%, 87%, 88%, 89%, 90%, 91%, 92%, 93%, 94%, 95%, 96%, 97%, 98%, or 99% identity to SEQ ID NO: 30. In some embodiments, the anti-CD19 CAR specifically binds to a B-lymphocyte antigen CD19 (CD19) protein. In some embodiments, the CD19 protein comprises the amino acid sequence of SEQ ID NO: 66.

In some embodiments, the CAR comprises transmembrane region, optionally a CD28 transmembrane domain. In some embodiments, the CD28 transmembrane domain comprises the amino acid sequence set forth in SEQ ID NO: 38.

In some embodiments, the anti-CD19 CAR further comprises a hinge domain between the extracellular antigen binding domain and the transmembrane domain. In some embodiments, the hinge domain comprises at least a portion of an IgG1 hinge domain. In some embodiments, the IgG1 hinge domain comprises an amino acid sequence set forth in SEQ ID NO: 32. In some embodiments, the IgG1 hinge domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 32.

In some embodiments, the intracellular signaling region further comprises a CD28 intracellular signaling domain. In some embodiments, the intracellular signaling region further comprises a CD3-zeta (CD3ζ) signaling domain. In some embodiments, the intracellular signaling region further comprises a CD28 intracellular signaling domain and a CD3-zeta signaling domain. In some embodiments, the CD28 intracellular signaling domain comprises an amino acid sequence set forth in SEQ ID NO: 5. In some embodiments, the CD28 intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 5. In some embodiments, the CD3-zeta intracellular signaling domain comprises an amino acid sequence set forth in SEQ ID NO: 13. In some embodiments, the CD3-zeta intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 13. In some embodiments, the intracellular signaling region comprises an amino acid sequence set forth in SEQ ID NO: 25. In some embodiments, the intracellular signaling region comprises an amino acid sequence having or having at least 85%, 86%, 87%, 88%, 89%, 90%, 91%, 92%, 93%, 94%, 95%, 96%, 97%, 98%, or 99% identity to SEQ ID NO: 25.

In some embodiments, the anti-CD19 CAR comprises an amino sequence set forth in SEQ ID NO: 42 or SEQ ID NO: 46. In some embodiments, the anti-CD19 CAR comprises an amino sequence set forth in SEQ ID NO: 69, SEQ ID NO: 70, or SEQ ID NO: 71.

Also provided herein are nucleic acid(s) encoding the anti-CD19 CAR(s) described herein.

In some embodiments, the nucleic acid encoding the anti-CD19 CAR comprises the nucleic acid sequence set forth in SEQ ID NO: 43 or SEQ ID NO: 47. In some embodiments, the nucleic acid further comprises a nucleic acid encoding an IL-15. In some embodiments, the IL-15 comprises the amino acid sequence set forth in SEQ ID NO: 22. In some embodiments, the IL-15 is encoded by a nucleic acid comprising SEQ ID NO: 23 or SEQ ID NO: 24. In some embodiments, the polynucleotide encodes a polyprotein comprising the CAR and the IL-15. In some embodiments, the polynucleotide further comprises a nucleic acid encoding a self-cleaving peptide, optionally a T2A self-cleaving peptide. In some embodiments, the CAR is joined to the IL-15 by the self-cleaving peptide. In some embodiments, the self-cleaving peptide is capable of inducing ribosomal skipping between the CAR and the IL-15. In some embodiments, the self-cleaving peptide comprises the sequence set forth in SEQ ID NO: 17. In some embodiments, the self-cleaving peptide is encoded by the sequence set forth in SEQ ID NO: 18.

In some embodiments, the polynucleotide further comprises a nucleic acid encoding a signal sequence. In some embodiments, the signal sequence comprises the amino acid sequence set forth in SEQ ID NO: 27. In some embodiments, the nucleic acid encoding the signal sequence comprises the nucleic acid sequence set forth in SEQ ID NO: 28.

In some embodiments, the polynucleotide encodes a polyprotein comprising the amino acid sequence set forth in SEQ ID NO: 44 or SEQ ID NO: 48. In some embodiments, the polynucleotide comprises the nucleic acid sequence set forth in SEQ ID NO: 43 or SEQ ID NO: 49.

Also provided herein are vector(s) comprising the polynucleotide(s) described herein. In some embodiments, the vector is a viral vector. In some embodiments, the viral vector is a retroviral vector or a lentiviral vector.

Also provided herein are cell(s) comprising the polynucleotide(s) and/or vector(s) described herein. Also provided herein are cell(s) expressing the chimeric antigen receptor(s) encoded by the polynucleotide(s) and/or vector(s) described herein. Also provided herein are cell(s) expressing the chimeric antigen receptor(s) and the IL-15 encoded by the polynucleotide(s) and/or vector(s) described herein.

In some embodiments, the cell is a lymphocyte. In some embodiments, the lymphocyte is a natural killer (NK) cell. In some embodiments, the lymphocyte is a T cell.

In some embodiments, the cell is a human cell. In some embodiments, the cell is a primary cell obtained from a subject. In some embodiments, the cell is a primary cell obtained from cord blood. In some embodiments, the cell comprises a KIR-B haplotype. In some embodiments, the cell express CD16 having the V/V polymorphism at F158.

Also provided herein are population(s) of cells comprising a plurality of the cells described herein. In some embodiments, at least 10%, 20%, 30%, 40%, 50%, 60%, 70%, 80%, 85%, 90%; 95%, 96%, 97%, 98%, or 99% of the cells comprise CAR(s) described herein, polynucleotide(s) described herein, and/or vector(s) described herein. In some embodiments, at least 10%, 20%, 30%, 40%, 50%, 60%, 70%, 80%, 85%, 90%; 95%, 96%, 97%, 98%, or 99% of the cells express a CAR and IL-15 encoded by the polynucleotide(s) and/or vector(s) described herein.

Also described herein are pharmaceutical composition(s) comprising the population(s) of cells described herein. In some embodiments, the pharmaceutical composition further comprises a pharmaceutically acceptable excipient. In some embodiments, the pharmaceutical composition further comprises (a) human albumin; (b) dextran; (c) glucose; (d) DMSO; and (e) a buffer. In some embodiments, the pharmaceutical composition comprises from 30 to 50 mg/mL human albumin. In some embodiments, the pharmaceutical composition comprises 50 mg/mL human albumin. In some embodiments, the pharmaceutical composition comprises 20 to 30 mg/mL dextran. In some embodiments, the pharmaceutical composition comprises 25 mg/mL dextran. In some embodiments, the dextran is Dextran 40. In some embodiments, the pharmaceutical composition comprises from 12 to 15 mg/mL glucose. In some embodiments, the pharmaceutical composition comprises 12.5 mg/mL glucose. In some embodiments, the pharmaceutical composition comprises less than 27.5 g/L glucose. In some embodiments, the pharmaceutical composition comprises from 50 to 60 mg/mL DMSO. In some embodiments, the pharmaceutical composition comprises 55 mg/mL DMSO. In some embodiments, the pharmaceutical composition comprises 40 to 60% v/v buffer. In some embodiments, the buffer is phosphate buffered saline. In some embodiments, the pharmaceutical composition comprises: (a) about 40 mg/mL human albumin; (b) about 25 mg/mL Dextran 40; (c) about 12.5 mg/mL glucose; (d) about 55 mg/mL DMSO; and (e) about 0.5 mL/mL phosphate buffered saline. In some embodiments, the pharmaceutical composition further comprises 0.5 mL/mL water.

Also provided herein are frozen vial(s) comprising the pharmaceutical composition(s) described herein.

Also provided herein are method(s) of treatment comprising administering cell(s) described herein, population(s) of cell(s) described herein, and/or composition(s) described herein to a subject having a disease or condition associated with CD19. Also provided herein are use(s) of the cell(s) described herein, population(s) of cell(s) described herein, and/or composition(s) described herein in the manufacture of a medicament for treating a disease or condition associated with CD19. Also provided herein are use(s) of the cell(s) described herein, population(s) of cell(s) described herein, and/or composition(s) described herein for treating a disease or condition associated with CD19.

In some embodiments, the disease or condition associated with CD19 is cancer. In some embodiments, the cancer is a CD19+ cancer. In some embodiments, the CD19 cancer is or comprises a hematopoietic neoplastic disorders expressing CD19. In some embodiments, the cancer is or comprises neoplastic cells of hematopoietic origin. In some embodiments, the cancer arises from a myeloid, lymphoid, or erythroid lineage, or a precursor cell thereof. In some embodiments, the cancer is or comprises a poorly-differentiated acute leukemia. In some embodiments, the cancer is or comprises a erythroblastic leukemia or an acute megakaryoblastic leukemia. In some embodiments, the cancer is a myeloma. In some embodiments, the cancer is or comprises an acute promyeloid leukemia (APML), acute myelogenous leukemia (AML) or chronic myelogenous leukemia (CML). In some embodiments, the cancer is a lymphoid malignancy or lymphoma. In some embodiments, the cancer is or comprises an acute lymphoblastic leukemia (ALL), chronic lymphocytic leukemia (CLL), prolymphocytic leukemia (PLL), hairy cell leukemia (HLL), or Waldenstrom's macroglobulinemia (WM). In some embodiments, the cancer is or comprises a non-Hodgkin lymphoma or a variant thereof, peripheral T cell lymphomas, adult T cell leukemia/lymphoma (ATL), cutaneous T-cell lymphoma (CTCL), large granular lymphocytic leukemia (LGF), Hodgkin's disease, or Reed-Sternberg disease.

In some embodiments, the method further comprises administering a lymphodepleting chemotherapy to the subject prior to treatment. In some embodiments, the lymphodepleting chemotherapy is non-myeloablative chemotherapy. In some embodiments, the lymphodepleting chemotherapy comprises treatment with at least one of cyclophosphamide and fludarabine. In some embodiments, the lymphodepleting chemotherapy comprises treatment with cyclophosphamide and fludarabine. In some embodiments, between 100 and 500 mg/m² cyclophosphamide is administered per day. In some embodiments, 250 mg/m² cyclophosphamide is administered per day. In some embodiments, 500 mg/m² cyclophosphamide is administered per day. In some embodiments, between 10 and 50 mg/m² of fludarabine is administered per day. In some embodiments, 30 mg/m² of fludarabine is administered per day.

In some embodiments, the method or use further comprises administering IL-2 to the subject. In some embodiments, the patient is administered 1×10⁶ IU/m² of IL-2. In some embodiments, the patient is administered 1×10⁷ IU of IL-2. In some embodiments, the patient is administered 6×10⁷ IU of IL-2. In some embodiments, administration of IL-2 occurs within 1-4 hours of administration of the cell(s) described herein, the population(s) of cells described herein, and/or the composition(s) described herein. In some embodiments, administration of IL-2 occurs at least 1-4 hours after the administration of the cell(s), population(s) of cells, and/or composition(s).

In some embodiments, the method or use comprises administering the cell(s), population(s) of cells, and/or composition(s) a plurality of times. In some embodiments, the method or use administering the cell(s), population(s) of cells, and/or composition(s) three, four times, or eight times. In some embodiments, the method or use administering the cell(s), population(s) of cells, and/or composition(s) every week, every two weeks, every three weeks, or every four weeks.

In some embodiments, the method or use further comprises administering rituximab to the subject. In some embodiments, the method or use further comprises administering obinutuzumab to the subject. In some embodiments, the method or use further comprises administering ofatumumab to the subject. In some embodiments, the method or use further comprises administering a checkpoint inhibitor to the subject. In some embodiments, the checkpoint inhibitor inhibits CTLA-4, PD-1, or PD-L1. In some embodiments, the checkpoint inhibitor is or comprises ipilimumab. In some embodiments, the checkpoint inhibitor is or comprises nivolumab. In some embodiments, the checkpoint inhibitor is or comprises pembrolizumab. In some embodiments, the checkpoint inhibitor is or comprises cemiplimab. In some embodiments, the checkpoint inhibitor is or comprises atezolizumab. In some embodiments, the checkpoint inhibitor is or comprises avelumab. In some embodiments, the checkpoint inhibitor is or comprises durvalumab.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative and are not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an exemplary embodiment of a method for NK cell expansion and stimulation.

FIG. 2 shows examples of different manufacturing schemes for master cell bank (MCB) and drug product (DP) manufacturing.

FIG. 3 shows phenotypes of expanded and stimulated population of NK cells.

FIG. 4 shows AB-202 purity and identity.

FIG. 5 shows AB-202 purity and identity. Bars, from left to right: AB101, TD3-2S, TD3-3S, TD10-25, TD10-3S.

FIG. 6 shows AB-202 purity and identity. Bars, from left to right: AB101, TD3-2S, TD3-3S, TD10-25, TD10-3S.

FIG. 7 shows AB-202 in Vitro Cytotoxicity.

FIG. 8 shows AB-202 in Vitro Cytotoxicity.

FIG. 9 shows AB-202 in Vitro Cytotoxicity.

FIG. 10 shows ADCC assay with Rituximab.

FIG. 11 shows ADCC assay with Obinutuzumab.

FIG. 12 shows cord blood natural killer cell (CBNK) ADCC using Rituximab.

FIG. 13 shows AB-202 ADCC using Rituximab.

FIG. 14 shows CBNK ADCC with Obinutuzumab.

FIG. 15 shows AB-202 ADCC with Obinutuzumab.

FIG. 16 shows degranulation of AB-202 cells in response to tumor cell lines.

FIG. 17 shows intracellular cytokine secretion of AB-202 cells in response to stimulation by various B cell tumor lines.

FIG. 18 shows intracellular cytokine secretion of AB-202 cells in response to stimulation by various B cell tumor lines.

FIG. 19 shows intracellular cytokine secretion of AB-202 cells in response to stimulation by various B cell tumor lines.

FIG. 20 shows an in vivo efficacy study design.

FIG. 21 shows anti-tumor activity of NK cells (CBNK (non-engineered cord blood NK cells) and AB-202)) in a mouse xenograft model.

FIG. 22 shows in vivo efficacy of AB-202 in an NSG xenograft model.

### DETAILED DESCRIPTION

Provided herein are, amongst other things, Natural Killer (NK) cells, e.g., CAR-NK cells, methods for producing the NK cells, pharmaceutical compositions comprising the NK cells, and methods of treating patients suffering, e.g., from cancer, with the NK cells.

### I. EXPANSION AND STIMULATION OF NATURAL KILLER CELLS

In some embodiments, natural killer cells are expanded and stimulated, e.g., by culturing and stimulation with feeder cells.

NK cells can be expanded and stimulated as described, for example, in US 2020/0108096 or WO 2020/101361, both of which are incorporated herein by reference in their entirety. Briefly, the source cells can be cultured on modified HuT-78 (ATCC® TIB-161™) cells that have been engineered to express 4-1BBL, membrane bound IL-21, and a mutant TNFα as described in US 2020/0108096.

Suitable NK cells can also be expanded and stimulated as described herein.

In some embodiments, NK cells are expanded and stimulated by a method comprising: (a) providing NK cells, e.g., a composition comprising NK cells, e.g., CD3(−) cells; and (b) culturing in a medium comprising feeder cells and/or stimulation factors, thereby producing a population of expanded and stimulated NK cells.

### A. Natural Killer Cell Sources

In some embodiments, the NK cell source is selected from the group consisting of peripheral blood, peripheral blood lymphocytes (PBLs), peripheral blood mononuclear cells (PBMCs), bone marrow, umbilical cord blood (cord blood), isolated NK cells, NK cells derived from induced pluripotent stem cells, NK cells derived from embryonic stem cells, and combinations thereof.

In some embodiments, the NK cell source is a single unit of cord blood.

In some embodiments, the natural killer cell source, e.g., single unit of cord blood, comprises from or from about 1×10⁷ to or to about 1×10⁹ total nucleated cells. In some embodiments, the natural killer cell source, e.g., single unit of cord blood, comprises from or from about 1×10⁸ to or to about 1.5×10⁸ total nucleated cells. In some embodiments, the natural killer cell source, e.g., single unit of cord blood, comprises 1×10⁸ total nucleated cells. In some embodiments, the natural killer cell source, e.g., single unit of cord blood, comprises about 1×10⁸ total nucleated cells. In some embodiments, the natural killer cell source, e.g., single unit of cord blood, comprises 1×10⁹ total nucleated cells. In some embodiments, the natural killer cell source, e.g., single unit of cord blood, comprises about 1×10⁹ total nucleated cells.

In some embodiments, the NK cell source, e.g., the cord blood unit, comprises from about 20% to about 80% CD16+ cells. In some embodiments, the NK cell source, e.g., the cord blood unit, comprises from or from about 20% to or to about 80%, from about 20% to or to about 70%, from about 20% to or to about 60%, from about 20% to or to about 50%, from about 20% to or to about 40%, from about 20% to or to about 30%, from about 30% to or to about 80%, from about 30% to or to about 70%, from about 30% to or to about 60%, from about 30% to or to about 50%, from about 30% to or to about 40%, from about 40% to or to about 80%, from about 40% to or to about 70%, from about 40% to or to about 60%, from about 40% to or to about 50%, from about 50% to or to about 80%, from about 50% to or to about 70%, from about 50% to or to about 60%, from about 60% to or to about 80%, from about 60% to or to about 70%, or from about 70% to or to about 80% CD16+ cells. In some embodiments, the NK cell source, e.g., the cord blood unit, comprises less than or equal to 80% CD16+ cells. Alternately, some NK cell sources may comprise CD16+ cells at a concentration of greater than 80%.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% MLG2A+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2C+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKG2D+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp46+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp30+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% DNAM-1+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% NKp44+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD25+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD62L+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD69+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CXCR3+ cells.

In some embodiments, the NK cell source, e.g., the cord blood unit, comprises less than or equal to 40%, e.g., less than or equal to 30%, e.g., less than or equal to 20%, e.g., less than or equal to 10%, e.g., less than or equal to 5% CD57+ cells.

In some embodiments, NK cells in the NK cell source comprise a KIR B allele of the KIR receptor family. See, e.g., Hsu et al., “The Killer Cell Immunoglobulin-Like Receptor (KIR) Genomic Region: Gene-Order, Haplotypes and Allelic Polymorphism,”Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010).

In some embodiments, NK cells in the NK cell source comprise the 158 V/V variant of CD16 (i.e. homozygous CD16 158V polymorphism). See, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).

In some embodiments, NK cells in the cell source comprises both the KIR B allele of the KIR receptor family and the 158 V/V variant of CD16. In some embodiments, the NK cells in the cell source are not genetically engineered. In some embodiments, the NK cells in the cell source do not comprise a CD16 transgene. In some embodiments, the NK cells in the cell source do not express an exogenous CD16 protein.

In some embodiments, the NK cell source is CD3(+) depleted. In some embodiments, the method comprises depleting the NK cell source of CD3(+) cells. In some embodiments, depleting the NK cell source of CD3(+) cells comprises contacting the NK cell source with a CD3 binding antibody or antigen binding fragment thereof. In some embodiments, the CD3 binding antibody or antigen binding fragment thereof is selected from the group consisting of OKT3, UCHT1, HIT3a, SK7, and fragments thereof. In some embodiments, the CD3 binding antibody or antigen binding fragment thereof is OKT3 or an antigen binding fragment thereof. In some embodiments, the antibody or antigen binding fragment thereof is attached to a bead, e.g., a magnetic bead. In some embodiments, the depleting the composition of CD3(+) cells comprises contacting the composition with a CD3 targeting antibody or antigen binding fragment thereof attached to a bead and removing the bead-bound CD3(+) cells from the composition. The composition can be depleted of CD3 cells by immunomagnetic selection, for example, using a CliniMACS T cell depletion set ((LS Depletion set (162-01) Miltenyi Biotec).

In some embodiments, the NK cell source CD56+ enriched, e.g., by gating on CD56 expression. In some embodiments, the NK cell source is both CD56+ enriched and CD3(+) depleted, e.g., by selecting for cells with CD56+CD3− expression. In some embodiments, the NK cell source comprises both the KIR B allele of the KIR receptor family and the 158 V/V variant of CD16 and is + enriched and CD3(+) depleted, e.g., by selecting for cells with CD56+CD3− expression.

### B. Feeder Cells

Disclosed herein are feeder cells for the expansion of NK cells. These feeder cells advantageously allow NK cells to expand to numbers suitable for the preparation of a pharmaceutical composition as discussed herein. In some cases, the feeder cells allow the expansion of NK cells without the loss of CD16 expression, which often accompanies cell expansion on other types of feeder cells or using other methods. In some cases, the feeder cells make the expanded NK cells more permissive to freezing such that a higher proportion of NK cells remain viable after a freeze/thaw cycle or such that the cells remain viable for longer periods of time while frozen. In some cases, the feeder cells allow the NK cells to retain high levels of cytotoxicity, including ADCC, extend survival, increase persistence, and enhance or retain high levels of CD16. In some cases, the feeder cells allow the NK cells to expand without causing significant levels of exhaustion or senescence.

Feeder cells can be used to stimulate the NK cells and help them to expand more quickly, e.g., by providing substrate, growth factors, and/or cytokines. NK cells can be stimulated using various types of feeder cells, including, but not limited to peripheral blood mononuclear cells (PBMC), Epstein-Barr virus-transformed B-lymphoblastoid cells (e.g., EBV-LCL), myelogenous leukemia cells (e.g., K562), and CD4(+) T cells (e.g., HuT), and derivatives thereof. In some embodiments, the feeder cells are inactivated, e.g., by γ-irradiation or mitomycin-c treatment. Suitable feeder cells for use in the methods described herein are described, for example, in US 2020/0108096, which is hereby incorporated by reference in its entirety.

In some embodiments, the feeder cell(s) are inactivated CD4(+) T cell(s). In some embodiments, the inactivated CD4(+) T cell(s) are HuT-78 cells (ATCC® TIB-161TM) or variants or derivatives thereof. In some embodiments, the HuT-78 derivative is H9 (ATCC@HTB-176™). In some embodiments, the inactivated CD4(+) T cell(s) express OX40L. In some embodiments, the inactivated CD4(+) T cell(s) are HuT-78 cells or variants or derivatives thereof that express OX40L (SEQ ID NO: 4) or a variant thereof. In some embodiments, the feeder cells are HuT-78 cells engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof. In some embodiments, the inactivated CD4(+) T cell(s) are HuT-78 (ATCC® TIB-161′) cells or variants or derivatives thereof that express an ortholog of OX40L, or variant thereof. In some embodiments, the feeder cells are HuT-78 cells engineered to express at least one gene selected from the group consisting of an 4-1BBL ortholog or variant thereof, a membrane bound IL-21 ortholog or variant thereof, and mutant TNFalpha ortholog, or variant thereof. In some embodiments, the feeder cells are HuT-78 cell(s) that express OX40L (SEQ ID NO: 4) and are engineered to express 4-1BBL (SEQ ID NO: 1), membrane bound IL-21 (SEQ ID NO: 2), and mutant TNFalpha (SEQ ID NO: 3) (“eHut-78 cells”) or variants or derivatives thereof.

In some embodiments, the feeder cells are expanded, e.g., from a frozen stock, before culturing with NK cells, e.g., as described in Example 2.

### C. Stimulating Factors

NK cells can also be stimulated using one or more stimulation factors other than feeder cells, e.g., signaling factors, in addition to or in place of feeder cells.

In some embodiments, the stimulating factor, e.g., signaling factor, is a component of the culture medium, as described herein. In some embodiments, the stimulating factor, e.g., signaling factor, is a supplement to the culture medium, as described herein.

In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof. In some embodiments, the cytokine is IL-2. In some embodiments, the cytokines are a combination of IL-2 and IL-15. In some embodiments, the cytokines are a combination of IL-2, IL-15, and IL-18. In some embodiments, the cytokines are a combination of IL-2, IL-18, and IL-21.

### D. Culturing

The NK cells can be expanded and stimulated by co-culturing an NK cell source and feeder cells and/or other stimulation factors. Suitable NK cell sources, feeder cells, and stimulation factors are described herein.

In some cases, the resulting population of expanded natural killer cells is enriched and/or sorted after expansion. In some cases, the resulting population of expanded natural killer cells is not enriched and/or sorted after expansion

Also described herein are compositions comprising the various culture compositions described herein, e.g., comprising NK cells. For example, a composition comprising a population of expanded cord blood-derived natural killer cells comprising a KIR-B haplotype and homozygous for a CD16 158V polymorphism and a plurality of engineered HuT78 cells.

Also described herein are vessels, e.g., vials, cryobags, and the like, comprising the resulting populations of expanded natural killer cells. In some cases, a plurality of vessels comprising portions of the resulting populations of expanded natural killer cells, e.g., at least 10, e.g., 20, 30, 40, 50, 60, 70, 80, 90, 100, 125, 150, 175, 200, 250, 300, 400, 500, 600, 700, 800, 900, 1000, 1100, or 1200 vessels.

Also described herein are bioreactors comprising the various culture compositions described herein, e.g., comprising NK cells. For example, a culture comprising natural killer cells from a natural killer cell source, e.g., as described herein, and feeder cells, e.g., as described herein. Also described herein are bioreactors comprising the resulting populations of expanded natural killer cells.

### 1. Culture Medium

Disclosed herein are culture media for the expansion of NK cells. These culture media advantageously allow NK cells to expand to numbers suitable for the preparation of a pharmaceutical composition as discussed herein. In some cases, the culture media allows NK cells to expand without the loss of CD16 expression that often accompanies cell expansion on other helper cells or in other media.

In some embodiments, the culture medium is a basal culture medium, optionally supplemented with additional components, e.g., as described herein.

In some embodiments, the culture medium, e.g., the basal culture medium, is a serum-free culture medium. In some embodiments, the culture medium, e.g., the basal culture medium, is a serum-free culture medium supplemented with human plasma and/or serum.

Suitable basal culture media include, but are not limited to, DMEM, RPMI 1640, MEM, DMEM/F12, Ham's F12, SCGM (CellGenix®, 20802-0500 or 20806-0500), LGM-3™ (Lonza, CC-3211), TexMACS™ (Miltenyi Biotec, 130-097-196), ALyS™ 505NK-AC (Cell Science and Technology Institute, Inc., 01600P02), ALyS™ 505NK-EX (Cell Science and Technology Institute, Inc., 01400P10), CTST™ AIM-VT™ SFM (ThermoFisher Scientific, A3830801), CTS™ OpTmizer™ (ThermoFisher Scientific, A1048501, ABS-001, StemXxVivo, SCGM, B0, HPLM, ExCellerate, X-VIVO 15, CTS expander, and combinations thereof.

The culture medium may comprise additional components, or be supplemented with additional components, such as growth factors, signaling factors, nutrients, antigen binders, and the like. Supplementation of the culture medium may occur by adding each of the additional component or components to the culture vessel either before, concurrently with, or after the medium is added to the culture vessel. The additional component or components may be added together or separately. When added separately, the additional components need not be added at the same time.

In some embodiments, the culture medium comprises plasma, e.g., human plasma. In some embodiments, the culture medium is supplemented with plasma, e.g., human plasma. In some embodiments, the plasma, e.g., human plasma, comprises an anticoagulant, e.g., trisodium citrate.

In some embodiments, the medium comprises and/or is supplemented with from or from about 0.5% to or to about 10% v/v plasma, e.g., human plasma. In some embodiments, the medium is supplemented with from or from about 0.5% to or to about 9%, from or from about 0.5% to or to about 8%, from or from about 0.5% to or to about 7%, from or from about 0.5% to or to about 6%, from or from about 0.5% to or to about 5%, from or from about 0.5% to or to about 4%, from or from about 0.5% to or to about 3%, from or from about 0.5% to or to about 2%, from or from about 0.5% to or to about 1%, from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 2% to or to about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 4% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v plasma, e.g., human plasma. In some embodiments, the culture medium comprises and/or is supplemented with from 0.8% to 1.2% v/v human plasma. In some embodiments, the culture medium comprises and/or is supplemented with 1.0% v/v human plasma. In some embodiments, the culture medium comprises and/or is supplemented with about 1.0% v/v human plasma.

In some embodiments, the culture medium comprises serum, e.g., human serum. In some embodiments, the culture medium is supplemented with serum, e.g., human serum. In some embodiments, the serum is inactivated, e.g., heat inactivated. In some embodiments, the serum is filtered, e.g., sterile-filtered.

In some embodiments, the culture medium comprises glutamine. In some embodiments, the culture medium is supplemented with glutamine. In some embodiments, the culture medium comprises and/or is supplemented with from or from about 2.0 to or to about 6.0 mM glutamine. In some embodiments, the culture medium comprises and/or is supplemented with from or from about 2.0 to or to about 5.5, from or from about 2.0 to or to about 5.0, from or from about 2.0 to or to about 4.5, from or from about 2.0 to or to about 4.0, from or from about 2.0 to or to about 3.5, from or from about 2.0 to or to about 3.0, from or from about 2.0 to or to about 2.5, from or from about 2.5 to or to about 6.0, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3.0 to or to about 6.0, from or from about 3.0 to or to about 5.5, from or from about 3.0 to or to about 5.0, from or from about 3.0 to or to about 4.5, from or from about 3.0 to or to about 4.0, from or from about 3.0 to or to about 3.5, from or from about 3.5 to or to about 6.0, from or from about 3.5 to or to about 5.5, from or from about 3.5 to or to about 5.0, from or from about 3.5 to or to about 4.5, from or from about 3.5 to or to about 4.0, from or from about 4.0 to or to about 6.0, from or from about 4.0 to or to about 5.5, from or from about 4.0 to or to about 5.0, from or from about 4.0 to or to about 4.5, from or from about 4.5 to or to about 6.0, from or from about 4.5 to or to about 5.5, from or from about 4.5 to or to about 5.0, from or from about 5.0 to or to about 6.0, from or from about 5.0 to or to about 5.5, or from or from about 5.5 to or to about 6.0 mM glutamine. In some embodiments, the culture medium comprises and/or is supplemented with from 3.2 mM glutamine to 4.8 mM glutamine. In some embodiments, the culture medium comprises and/or is supplemented with 4.0 mM glutamine. In some embodiments, the culture medium comprises and/or is supplemented with about 4.0 mM glutamine.

In some embodiments, the culture medium comprises one or more cyotkines. In some embodiments, the culture medium is supplemented with one or more cyotkines. In some embodiments, the cytokine is selected from IL-2, IL-12, IL-15, IL-18, IL-21, and combinations thereof.

In some embodiments, the culture medium comprises and/or is supplemented with IL-2. In some embodiments, the culture medium comprises and/or is supplemented with from or from about 150 to or to about 2,500 IU/mL IL-2. In some embodiments, the culture medium comprises and/or is supplemented with from or from about 200 to or to about 2,250, from or from about 200 to or to about 2,000, from or from about 200 to or to about 1,750, from or from about 200 to or to about 1,500, from or from about 200 to or to about 1,250, from or from 200 to or to about 1,000, from or from about 200 to or to about 750, from or from about 200 to or to about 500, from or from about 200 to or to about 250, from or from about 250 to or to about 2,500, from or from about 250 to or to about 2,250, from or from about 250 to or to about 2,000, from or from about 250 to or to about 1,750, from or from about 250 to or to about 1,500, from or from about 250 to or to about 1,250, from or from about 250 to or to about 1,000, from or from about 250 to or to about 750, from or from about 250 to or to about 500, from or from about 500 to or to about 2,500, from or from about 500 to or to about 2,250, from or from about 500 to or to about 2,000, from or from about 500 to or to about 1,750, from or from about 500 to or to about 1,500, from or from about 500 to or to about 1,250, from or from about 500 to or to about 1,000, from or from about 500 to or to about 750, from or from about 750 to or to about 2,250, from or from about 750 to or to about 2,000, from or from about 750 to or to about 1,750, from or from about 750 to or to about 1,500, from or from about 750 to or to about 1,250, from or from about 750 to or to about 1,000, from or from about 1,000 to or to about 2,500, from or from about 1,000 to or to about 2,250, from or from about 1,000 to or to about 2,000, from or from about 1,000 to or to about 1,750, from or from about 1,000 to or to about 1,500, from or from about 1,000 to or to about 1,250, from or from about 1,250 to or to about 2,500, from or from about 1,250 to or to about 2,250, from or from about 1,250 to or to about 2,000, from or from about 1,250 to or to about 1,750, from or from about 1,250 to or to about 1,500, from or from about 1,500 to or to about 2,500, from or from about 1,500 to or to about 2,250, from or from about 1,500 to or to about 2,000, from or from about 1,500 to or to about 1,750, from or from about 1,750 to or to about 2,500, from or from about 1,750 to or to about 2,250, from or from about 1,750 to or to about 2,000, from or from about 2,000 to or to about 2,500, from or from about 2,000 to or to about 2,250, or from or from about 2,250 to or to about 2,500 IU/mL IL-2.

In some embodiments, the culture medium comprises and/or is supplemented with from 64 μg/L to 96 μg/L IL-2. In some embodiments, the culture medium comprises and/or is supplemented with 80 μg/L IL-2 (approximately 1,333 IU/mL). In some embodiments, the culture medium comprises and/or is supplemented with about 80 μg/L IL-2. In some embodiments, the culture medium comprises and/or is supplemented with from 10 μg/L to 100 μg/L IL-21. In some embodiments, the culture medium comprises and/or is supplemented with 20 μg/L IL-21. In some embodiments, the culture medium comprises and/or is supplemented with about 20 μg/L. In some embodiments, the culture medium comprises and/or is supplemented with a combination of IL-2 and IL-15. In some embodiments, the culture medium comprises and/or is supplemented with a combination of IL-2, IL-15, and IL-18. In some embodiments, the culture medium comprises and/or is supplemented with a combination of IL-2, IL-18, and IL-21.

In some embodiments, the culture medium comprises and/or is supplemented with glucose. In some embodiments, the culture medium comprises and/or is supplemented with from or from about 0.5 to or to about 3.5 g/L glucose. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L glucose. In some embodiments, the culture medium comprises and/or is supplemented with from 1.6 to 2.4 g/L glucose. In some embodiments, the culture medium comprises and/or is supplemented with 2.0 g/L glucose. In some embodiments, the culture medium comprises about 2.0 g/L glucose.

In some embodiments, the culture medium comprises and/or is supplemented with sodium pyruvate. In some embodiments, the culture medium comprises and/or is supplemented with from or from about 0.1 to or to about 2.0 mM sodium pyruvate. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 mM sodium pyruvate. In some embodiments, the culture medium comprises from 0.8 to 1.2 mM sodium pyruvate. In some embodiments, the culture medium comprises 1.0 mM sodium pyruvate. In some embodiments, the culture medium comprises about 1.0 mM sodium pyuruvate.

In some embodiments, the culture medium comprises and/or is supplemented with sodium hydrogen carbonate. In some embodiments, the culture medium comprises and/or is supplemented with from or from about 0.5 to or to about 3.5 g/L sodium hydrogen carbonate. In some embodiments, the culture medium comprises and/or is supplemented with from or from about 0.5 to or to about 3.0, from or from about 0.5 to or to about 2.5, from or from about 0.5 to or to about 2.0, from or from about 0.5 to or to about 1.5, from or from about 0.5 to or to about 1.0, from or from about 1.0 to or to about 3.0, from or from about 1.0 to or to about 2.5, from or from about 1.0 to or to about 2.0, from or from about 1.0 to or to about 1.5, from or from about 1.5 to or to about 3.0, from or from about 1.5 to or to about 2.5, from or from about 1.5 to or to about 2.0, from or from about 2.0 to or to about 3.0, from or from about 2.0 to or to about 2.5, or from or from about 2.5 to or to about 3.0 g/L sodium hydrogen carbonate. In some embodiments, the culture medium comprises and/or is supplemented with from 1.6 to 2.4 g/L sodium hydrogen carbonate. In some embodiments, the culture medium comprises and/or is supplemented with 2.0 g/L sodium hydrogen carbonate. In some embodiments, the culture medium comprises about 2.0 g/L sodium hydrogen carbonate.

In some embodiments, the culture medium comprises and/or is supplemented with albumin, e.g., human albumin, e.g., a human albumin solution described herein. In some embodiments, the culture medium comprises and/or is supplemented with from or from about 0.5% to or to about 3.5% v/v of a 20% albumin solution, e.g., a 20% human albumin solution. In some embodiments, the culture medium comprises and/or is supplemented with from or from about 0.5% to or to about 3.0%, from or from about 0.5% to or to about 2.5%, from or from about 0.5% to or to about 2.0%, from or from about 0.5% to or to about 1.5%, from or from about 0.5% to or to about 1.0%, from or from about 1.0% to or to about 3.0%, from or from about 1.0% to or to about 2.5%, from or from about 1.0% to or to about 2.0%, from or from about 1.0% to or to about 1.5%, from or from about 1.5% to or to about 3.0%, from or from about 1.5% to or to about 2.5%, from or from about 1.5% to or to about 2.0%, from or from about 2.0% to or to about 3.0%, from or from about 2.0% to or to about 2.5%, or from or from about 2.5% to or to about 3.0% v/v of a 20% albumin solution, e.g., a 20% human albumin solution. In some embodiments, the culture medium comprises and/or is supplemented with from 1.6% to 2.4% v/v of a 20% albumin solution, e.g., a 20% human albumin solution. In some embodiments, the culture medium comprises and/or is supplemented with 2.0% v/v of a 20% albumin solution, e.g., a 20% human albumin solution. In some embodiments, the culture medium comprises about 2.0% v/v of a 20% albumin solution, e.g., a 20% human albumin solution.

In some embodiments, the culture medium comprises and/or is supplemented with from or from about 2 to or to about 6 g/L albumin, e.g., human albumin. In some embodiments, the culture medium comprises and/or is supplemented with from or from about 2 to or to about 5.5, from or from about 2 to or to about 5.0, from or from about 2 to or to about 4.5, from or from about 2 to or to about 4, from or from about 2 to or to about 3.5, from or from about 2 to or to about 3, from or from about 2 to or to about 2.5, from or from about 2.5 to or to about 6, from or from about 2.5 to or to about 5.5, from or from about 2.5 to or to about 5.5, from or from about 2.5 to or to about 5.0, from or from about 2.5 to or to about 4.5, from or from about 2.5 to or to about 4.0, from or from about 2.5 to or to about 3.5, from or from about 2.5 to or to about 3.0, from or from about 3 to or to about 6, from or from about 3 to or to about 5.5, from or from about 3 to or to about 5, from or from about 3 to or to about 4.5, from or from about 3 to or to about 4, from or from about 3 to or to about 3.5, from or from about 3.5 to or to about 6, from or from about 3.5 to or to about 5.5, from or from about 3.5 to or to about 5, from or from about 3.5 to or to about 4.5, from or from about 3.5 to or to about 4, from or from about 4 to or to about 6, from or from about 4 to or to about 5.5, from or from about 4 to or to about 5, from or from about 4 to or to about 4.5, from or from about 4.5 to or to about 6, from or from about 4.5 to or to about 5.5, from or from about 4.5 to or to about 5, from or from about 5 to or to about 6, from or from about 5 to or to about 5.5, or from or from about 5.5 to or to about 6 g/L albumin, e.g., human albumin. In some embodiments, the culture medium comprises and/or is supplemented with from 3.2 to 4.8 g/L albumin, e.g., human albumin. In some embodiments, the culture medium comprises 4 g/L albumin, e.g., human albumin. In some embodiments, the culture medium comprises about 4 g/L albumin, e.g., human albumin

In some embodiments, the culture medium is supplemented with Poloxamer 188. In some embodiments, the culture medium comprises and/or is supplemented with from or from about 0.1 to or to about 2.0 g/L Poloxamer 188. In some embodiments, the culture medium comprises and/or is supplemented with from or from about 0.1 to or to about 1.8, from or from about 0.1 to or to about 1.6, from or from about 0.1 to or to about 1.4, from or from about 0.1 to or to about 1.2, from or from about 0.1 to or to about 1.0, from or from about 0.1 to or to about 0.8, from or from about 0.1 to or to about 0.6, from or from about 0.1 to or to about 0.4, from or from about 0.1 to or to about 0.2, from or from about 0.2 to or to about 2.0, from or from about 0.2 to or to about 1.8, from or from about 0.2 to or to about 1.6, from or from about 0.2 to or to about 1.4, from or from about 0.2 to or to about 1.2, from or from about 0.2 to or to about 1.0, from or from about 0.2 to or to about 0.8, from or from about 0.2 to or to about 0.6, from or from about 0.2 to or to about 0.4, from or from about 0.4 to or to about 2.0, from or from about 0.4 to or to about 1.8, from or from about 0.4 to or to about 1.6, from or from about 0.4 to or to about 1.4, from or from about 0.4 to or to about 1.2, from or from about 0.4 to or to about 1.0, from or from about 0.4 to or to about 0.8, from or from about 0.4 to or to about 0.6, from or from about 0.6 to or to about 2.0, from or from about 0.6 to or to about 1.8, from or from about 0.6 to or to about 1.6, from or from about 0.6 to or to about 1.4, from or from about 0.6 to or to about 1.2, from or from about 0.6 to or to about 1.0, from or form about 0.6 to or to about 0.8, from or from about 0.8 to or to about 2.0, from or from about 0.8 to or to about 1.8, from or from about 0.8 to or to about 1.6, from or from about 0.8 to or to about 1.4, from or from about 0.8 to or to about 1.4, from or from about 0.8 to or to about 1.2, from or from about 0.8 to or to about 1.0, from or from about 1.0 to or to about 2.0, from or from about 1.0 to or to about 1.8, from or from about 1.0 to or to about 1.6, from or from about 1.0 to or to about 1.4, from or from about 1.0 to or to about 1.2, from or from about 1.2 to or to about 2.0, from or from about 1.2 to or to about 1.8, from or from about 1.2 to or to about 1.6, from or from about 1.2 to or to about 1.4, from or from about 1.4 to or to about 2.0, from or from about 1.4 to or to about 1.8, from or from about 1.4 to or to about 1.6, from or from about 1.6 to or to about 2.0, from or from about 1.6 to or to about 1.8, or from or from about 1.8 to or to about 2.0 g/L Poloxamer 188. In some embodiments, the culture medium comprises from 0.8 to 1.2 g/L Poloxamer 188. In some embodiments, the culture medium comprises 1.0 g/L Poloxamer 188. In some embodiments, the culture medium comprises about 1.0 g/L Poloxamer 188.

In some embodiments, the culture medium comprises and/or is supplemented with one or more antibiotics.

A first exemplary culture medium is set forth in Table 1.

TABLE 1
Exemplary Culture Medium #1

| | | |
|---|---|---|
| | Exemplary | Exemplary |
| Component | Concentration Range | Concentration |

| | | |
|---|---|---|
| CellgroSCGM | undiluted | undiluted |
| liquid medium | | |

| | | | | |
|---|---|---|---|---|
| Human Plasma | 0.8-1.2% | (v/v) | 1.0% | v/v |
| Glutamine | 3.2-4.8 | mM | 4.0 | mM |
| IL-2 | 64-96 | μg/L | 80 | μg/L |

A second exemplary culture medium is set forth in Table 2.

TABLE 2
Exemplary Culture Medium #2

| | | |
|---|---|---|
| | Exemplary | Exemplary |
| Component | Concentration Range | Concentration |

| | | | | |
|---|---|---|---|---|
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen | 1.6-2.4 | g/L | 2.0 | g/L |
| Carbonate | | | | |
| IL-2 | 64-96 | μg/L | 80 | μg/L |
| Albumin 20% solution | 1.6-2.5% | v/v | 2.0% | v/v |
| | (3.2 to 4.8 | g/L) | (4.0 | g/L) |
| Poloxamer 188 | 0.8-1.2 | g/L | 1.0 | g/L |

A third exemplary culture medium is set forth in Table 3.

TABLE 3
Exemplary Culture Medium #3

| | | |
|---|---|---|
| | Exemplary | Exemplary |
| Component | Concentration Range | Concentration |

| | | | | |
|---|---|---|---|---|
| RPMI1640 | 7.6-13.2 | g/L | 10.4 | g/L |
| Human Plasma | 0.8-1.2% | (v/v) | 1.0% | v/v |
| Glucose | 1.6-2.4 | g/L | 2.0 | g/L |
| Glutamine | 3.2-4.8 | mM | 4.0 | mM |
| Sodium Pyruvate | 0.8-1.2 | mM | 1.0 | mM |
| Sodium Hydrogen | 1.6-2.4 | g/L | 2.0 | g/L |
| Carbonate | | | | |
| IL-2 | 64-96 | μg/L | 80 | μg/L |
| Albumin 20% solution | 1.6-2.5% | v/v | 2.0% | v/v |
| | (3.2 to 4.8 | g/L) | (4.0 | g/L) |
| Poloxamer 188 | 0.8-1.2 | g/L | 1.0 | g/L |
| IL-21 | 10-30 | μg/L | 20 | μg/L |

### 2. CD3 Binding Antibodies

In some embodiments, the culture medium comprises and/or is supplemented with a CD3 binding antibody or antigen binding fragment thereof. In some embodiments, the CD3 binding antibody or antigen binding fragment thereof is selected from the group consisting of OKT3, UCHT1, and HIT3a, or variants thereof. In some embodiments, the CD3 binding antibody or antigen binding fragment thereof is OKT3 or an antigen binding fragment thereof.

In some embodiments, the CD3 binding antibody or antigen binding fragment thereof and feeder cells are added to the culture vessel before addition of NK cells and/or culture medium.

In some embodiments, the culture medium comprises and/or is supplemented with from or from about 5 ng/mL to or to about 15 ng/mL OKT3. In some embodiments, the culture medium comprises and/or is supplemented with from or from about 5 to or to about 12.5, from or from about 5 to or to about 10, from or from about 5 to or to about 7.5, from or from about 7.5 to or to about 15, from or from about 7.5 to or to about 12.5, from or from about 7.5 to or to about 10, from or from about 10 to or to about 15, from or from about 10 to or to about 12.5, or from or from about 12.5 to or to about 15 ng/mL OKT3. In some embodiments, the culture medium comprises and/or is supplemented with 10 ng/mL OKT3. In some embodiments, the culture medium comprises and/or is supplemented with about 10 ng/mL OKT3.

### 3. Culture Vessels

A number of vessels are consistent with the disclosure herein. In some embodiments, the culture vessel is selected from the group consisting of a flask, a bottle, a dish, a multiwall plate, a roller bottle, a bag, and a bioreactor.

In some embodiments, the culture vessel is treated to render it hydrophilic. In some embodiments, the culture vessel is treated to promote attachment and/or proliferation. In some embodiments, the culture vessel surface is coated with serum, collagen, laminin, gelatin, poy-L-lysine, fibronectin, extracellular matrix proteins, and combinations thereof.

In some embodiments, different types of culture vessels are used for different stages of culturing.

In some embodiments, the culture vessel has a volume of from or from about 100 mL to or to about 1,000 L. In some embodiments, the culture vessel has a volume of or about 125 mL, of or about 250 mL, of or about 500 mL, of or about 1 L, of or about 5 L, of about 10 L, or of or about 20 L.

In some embodiments, the culture vessel is a bioreactor. In some embodiments, the bioreactor is a rocking bed (wave motion) bioreactor. In some embodiments, the bioreactor is a stirred tank bioreactor. In some embodiments, the bioreactor is a rotating wall vessel. In some embodiments, the bioreactor is a perfusion bioreactor. In some embodiments, the bioreactor is an isolation/expansion automated system. In some embodiments, the bioreactor is an automated or semi-automated bioreactor. In some embodiments, the bioreactor is a disposable bag bioreactor.

In some embodiments, the bioreactor has a volume of from about 100 mL to about 1,000 L. In some embodiments, the bioreactor has a volume of from about 10 L to about 1,000 L. In some embodiments, the bioreactor has a volume of from about 100 L to about 900 L. In some embodiments, the bioreactor has a volume of from about 10 L to about 800 L. In some embodiments, the bioreactor has a volume of from about 10 L to about 700 L, about 10 L to about 600 L, about 10 L to about 500 L, about 10 L to about 400 L, about 10 L to about 300 L, about 10 L to about 200 L, about 10 L to about 100 L, about 10 L to about 90 L, about 10 L to about 80 L, about 10 L to about 70 L, about 10 L to about 60 L, about 10 L to about 50 L, about 10 L to about 40 L, about 10 L to about 30 L, about 10 L to about 20 L, about 20 L to about 1,000 L, about 20 L to about 900 L, about 20 L to about 800 L, about 20 L to about 700 L, about 20 L to about 600 L, about 20 L to about 500 L, about 20 L to about 400 L, about 20 L to about 300 L, about 20 L to about 200 L, about 20 L to about 100 L, about 20 L to about 90 L, about 20 L to about 80 L, about 20 L to about 70 L, about 20 L to about 60 L, about 20 L to about 50 L, about 20 L to about 40 L, about 20 L to about 30 L, about 30 L to about 1,000 L, about 30 L to about 900 L, about 30 L to about 800 L, about 30 L to about 700 L, about 30 L to about 600 L, about 30 L to about 500 L, about 30 L to about 400 L, about 30 L to about 300 L, about 30 L to about 200 L, about 30 L to about 100 L, about 30 L to about 90 L, about 30 L to about 80 L, about 30 L to about 70 L, about 30 L to about 60 L, about 30 L to about 50 L, about 30 L to about 40 L, about 40 L to about 1,000 L, about 40 L to about 900 L, about 40 L to about 800 L, about 40 L to about 700 L, about 40 L to about 600 L, about 40 L to about 500 L, about 40 L to about 400 L, about 40 L to about 300 L, about 40 L to about 200 L, about 40 L to about 100 L, about 40 L to about 90 L, about 40 L to about 80 L, about 40 L to about 70 L, about 40 L to about 60 L, about 40 L to about 50 L, about 50 L to about 1,000 L, about 50 L to about 900 L, about 50 L to about 800 L, about 50 L to about 700 L, about 50 L to about 600 L, about 50 L to about 500 L, about 50 L to about 400 L, about 50 L to about 300 L, about 50 L to about 200 L, about 50 L to about 100 L, about 50 L to about 90 L, about 50 L to about 80 L, about 50 L to about 70 L, about 50 L to about 60 L, about 60 L to about 1,000 L, about 60 L to about 900 L, about 60 L to about 800 L, about 60 L to about 700 L, about 60 L to about 600 L, about 60 L to about 500 L, about 60 L to about 400 L, about 60 L to about 300 L, about 60 L to about 200 L, about 60 L to about 100 L, about 60 L to about 90 L, about 60 L to about 80 L, about 60 L to about 70 L, about 70 L to about 1,000 L, about 70 L to about 900 L, about 70 L to about 800 L, about 70 L to about 700 L, about 70 L to about 600 L, about 70 L to about 500 L, about 70 L to about 400 L, about 70 L to about 300 L, about 70 L to about 200 L, about 70 L to about 100 L, about 70 L to about 90 L, about 70 L to about 80 L, about 80 L to about 1,000 L, about 80 L to about 900 L, about 80 L to about 800 L, about 80 L to about 700 L, about 80 L to about 600 L, about 80 L to about 500 L, about 80 L to about 400 L, about 80 L to about 300 L, about 80 L to about 200 L, about 80 L to about 100 L, about 80 L to about 90 L, about 90 L to about 1,000 L, about 90 L to about 900 L, about 90 L to about 800 L, about 90 L to about 700 L, about 90 L to about 600 L, about 90 L to about 500 L, about 90 L to about 400 L, about 90 L to about 300 L, about 90 L to about 200 L, about 90 L to about 100 L, about 100 L to about 1,000 L, about 100 L to about 900 L, about 100 L to about 800 L, about 100 L to about 700 L, about 100 L to about 600 L, about 100 L to about 500 L, about 100 L to about 400 L, about 100 L to about 300 L, about 100 L to about 200 L, about 200 L to about 1,000 L, about 200 L to about 900 L, about 200 L to about 800 L, about 200 L to about 700 L, about 200 L to about 600 L, about 200 L to about 500 L, about 200 L to about 400 L, about 200 L to about 300 L, about 300 L to about 1,000 L, about 300 L to about 900 L, about 300 L to about 800 L, about 300 L to about 700 L, about 300 L to about 600 L, about 300 L to about 500 L, about 300 L to about 400 L, about 400 L to about 1,000 L, about 400 L to about 900 L, about 400 L to about 800 L, about 400 L to about 700 L, about 400 L to about 600 L, about 400 L to about 500 L, about 500 L to about 1,000 L, about 500 L to about 900 L, about 500 L to about 800 L, about 500 L to about 700 L, about 500 L to about 600 L, about 600 L to about 1,000 L, about 600 L to about 900 L, about 600 L to about 800 L, about 600 L to about 700 L, about 700 L to about 1,000 L, about 700 L to about 900 L, about 700 L to about 800 L, about 800 L to about 1,000 L, about 800 L to about 900 L, or about 900 L to about 1,000 L. In some embodiments, the bioreactor has a volume of about 50 L.

In some embodiments, the bioreactor has a volume of from 100 mL to 1,000 L. In some embodiments, the bioreactor has a volume of from 10 L to 1,000 L. In some embodiments, the bioreactor has a volume of from 100 L to 900 L. In some embodiments, the bioreactor has a volume of from 10 L to 800 L. In some embodiments, the bioreactor has a volume of from 10 L to 700 L, 10 L to 600 L, 10 L to 500 L, 10 L to 400 L, 10 L to 300 L, 10 L to 200 L, 10 L to 100 L, 10 L to 90 L, 10 L to 80 L, 10 L to 70 L, 10 L to 60 L, 10 L to 50 L, 10 L to 40 L, 10 L to 30 L, 10 L to 20 L, 20 L to 1,000 L, 20 L to 900 L, 20 L to 800 L, 20 L to 700 L, 20 L to 600 L, 20 L to 500 L, 20 L to 400 L, 20 L to 300 L, 20 L to 200 L, 20 L to 100 L, 20 L to 90 L, 20 L to 80 L, 20 L to 70 L, 20 L to 60 L, 20 L to 50 L, 20 L to 40 L, 20 L to 30 L, 30 L to 1,000 L, 30 L to 900 L, 30 L to 800 L, 30 L to 700 L, 30 L to 600 L, 30 L to 500 L, 30 L to 400 L, 30 L to 300 L, 30 L to 200 L, 30 L to 100 L, 30 L to 90 L, 30 L to 80 L, 30 L to 70 L, 30 L to 60 L, 30 L to 50 L, 30 L to 40 L, 40 L to 1,000 L, 40 L to 900 L, 40 L to 800 L, 40 L to 700 L, 40 L to 600 L, 40 L to 500 L, 40 L to 400 L, 40 L to 300 L, 40 L to 200 L, 40 L to 100 L, 40 L to 90 L, 40 L to 80 L, 40 L to 70 L, 40 L to 60 L, 40 L to 50 L, 50 L to 1,000 L, 50 L to 900 L, 50 L to 800 L, 50 L to 700 L, 50 L to 600 L, 50 L to 500 L, 50 L to 400 L, 50 L to 300 L, 50 L to 200 L, 50 L to 100 L, 50 L to 90 L, 50 L to 80 L, 50 L to 70 L, 50 L to 60 L, 60 L to 1,000 L, 60 L to 900 L, 60 L to 800 L, 60 L to 700 L, 60 L to 600 L, 60 L to 500 L, 60 L to 400 L, 60 L to 300 L, 60 L to 200 L, 60 L to 100 L, 60 L to 90 L, 60 L to 80 L, 60 L to 70 L, 70 L to 1,000 L, 70 L to 900 L, 70 L to 800 L, 70 L to 700 L, 70 L to 600 L, 70 L to 500 L, 70 L to 400 L, 70 L to 300 L, 70 L to 200 L, 70 L to 100 L, 70 L to 90 L, 70 L to 80 L, 80 L to 1,000 L, 80 L to 900 L, 80 L to 800 L, 80 L to 700 L, 80 L to 600 L, 80 L to 500 L, 80 L to 400 L, 80 L to 300 L, 80 L to 200 L, 80 L to 100 L, 80 L to 90 L, 90 L to 1,000 L, 90 L to 900 L, 90 L to 800 L, 90 L to 700 L, 90 L to 600 L, 90 L to 500 L, 90 L to 400 L, 90 L to 300 L, 90 L to 200 L, 90 L to 100 L, 100 L to 1,000 L, 100 L to 900 L, 100 L to 800 L, 100 L to 700 L, 100 L to 600 L, 100 L to 500 L, 100 L to 400 L, 100 L to 300 L, 100 L to 200 L, 200 L to 1,000 L, 200 L to 900 L, 200 L to 800 L, 200 L to 700 L, 200 L to 600 L, 200 L to 500 L, 200 L to 400 L, 200 L to 300 L, 300 L to 1,000 L, 300 L to 900 L, 300 L to 800 L, 300 L to 700 L, 300 L to 600 L, 300 L to 500 L, 300 L to 400 L, 400 L to 1,000 L, 400 L to 900 L, 400 L to 800 L, 400 L to 700 L, 400 L to 600 L, 400 L to 500 L, 500 L to 1,000 L, 500 L to 900 L, 500 L to 800 L, 500 L to 700 L, 500 L to 600 L, 600 L to 1,000 L, 600 L to 900 L, 600 L to 800 L, 600 L to 700 L, 700 L to 1,000 L, 700 L to 900 L, 700 L to 800 L, 800 L to 1,000 L, 800 L to 900 L, or 900 L to 1,000 L. In some embodiments, the bioreactor has a volume of 50 L.

### 4. Cell Expansion and Stimulation

In some embodiments, the natural killer cell source, e.g., single unit of cord blood, is co-cultured with feeder cells to produce expanded and stimulated NK cells.

In some embodiments, the co-culture is carried out in a culture medium described herein, e.g., exemplary culture medium #1 (Table 1), exemplary culture medium #2 (Table 2), or exemplary culture medium #3 (Table 3).

In some embodiments, the natural killer cell source, e.g., single unit of cord blood, comprises from or from about 1×10⁷ to or to about 1×10⁹ total nucleated cells prior to expansion. In some embodiments, the natural killer cell source, e.g., single unit of cord blood, comprises from or from about 1×10⁸ to or to about 1.5×10⁸ total nucleated cells prior to expansion. In some embodiments, the natural killer cell source, e.g., single unit of cord blood, comprises 1×10⁸ total nucleated cells prior to expansion. In some embodiments, the natural killer cell source, e.g., single unit of cord blood, comprises about 1×10⁸ total nucleated cells prior to expansion. In some embodiments, the natural killer cell source, e.g., single unit of cord blood, comprises 1×10⁹ total nucleated cells prior to expansion. In some embodiments, the natural killer cell source, e.g., single unit of cord blood, comprises about 1×10⁹ total nucleated cells prior to expansion.

In some embodiments, cells from the co-culture of the natural killer cell source, e.g., single unit of cord blood and feeder cells are harvested and frozen, e.g., in a cryopreservation composition described herein. In some embodiments, the frozen cells from the co-culture are an infusion-ready drug product. In some embodiments, the frozen cells from the co-culture are used as a master cell bank (MCB) from which to produce an infusion-ready drug product, e.g., through one or more additional co-culturing steps, as described herein. Thus, for example, a natural killer cell source can be expanded and stimulated as described herein to produce expanded and stimulated NK cells suitable for use in an infusion-ready drug product without generating any intermediate products. A natural killer cell source can also be expanded and stimulated as described herein to produce an intermediate product, e.g., a first master cell bank (MCB). The first MCB can be used to produce expanded and stimulated NK cells suitable for use in an infusion-ready drug product, or, alternatively, be used to produce another intermediate product, e.g., a second MCB. The second MCB can be used to produce expanded and stimulated NK cells suitable for an infusion-ready drug product, or alternatively, be used to produce another intermediate product, e.g., a third MCB, and so on.

In some embodiments, the ratio of feeder cells to cells of the natural killer cell source or MCB cells inoculated into the co-culture is from or from about 1:1 to or to about 4:1. In some embodiments, the ratio of feeder cells to cells of the natural killer cell source or MCB cells is from or from about 1:1 to or to about 3.5:1, from or from about 1:1 to or to about 3:1, from or from about 1:1 to or to about 2.5:1, from or from about 1.1 to or to about 2:1, from or from about 1:1 to or to about 1.5:1, from or from about 1.5:1 to or to about 4:1, from or from about 1.5:1 to or to about 3.5:1, from or from about 1.5:1 to or to about 3:1, from or from about 1.5:1 to or to about 2.5:1, from or from about 1.5:1 to or to about 2:1, from or from about 2:1 to or to about 4:1, from or from about 2:1 to or to about 3.5:1, from or from about 2:1 to or to about 3:1, from or from about 2:1 to or to about 2.5:1, from or from about 2.5:1 to or to about 4:1, from or from about 2.5:1 to or to about 3.5:1, from or from about 2.5:1 to or to about 3:1, from or from about 3:1 to or to about 4:1, from or from about 3:1 to or to about 3.5:1, or from or from about 3.5:1 to or to about 4:1. In some embodiments, the ratio of feeder cells to cells of the natural killer cell source or MCB inoculated into the co-culture is 2.5:1. In some embodiments, the ratio of feeder cells to cells of the natural killer cell source or MCB inoculated into the co-culture is about 2.5:1.

In some embodiments, the co-culture is carried out in a disposable culture bag, e.g., a 1 L disposable culture bag. In some embodiments, the co-culture is carried out in a bioreactor, e.g., a 50 L bioreactor. In some embodiments, culture medium is added to the co-culture after the initial inoculation.

In some embodiments, the co-culture is carried out for 1, 2, 3, 4, 5, 6, 7, 8, 9, 10, 11, 12, 13, 14, 15, 16, 17, 18, 19, 20, 21, 22, 23, 24 or more days. In some embodiments, the co-culture is carried out for a maximum of 16 days.

In some embodiments, the co-culture is carried out at 37° C. or about 37° C. In some embodiments, the co-culture is carried out at pH 7.9 or about pH 7.9. In some embodiments, the co-culture is carried out at a dissolved oxygen (DO) level of 50% or more.

In some embodiments, exemplary culture medium #1 (Table 1) is used to produce a MCB and exemplary culture medium #2 (Table 2) is used to produce cells suitable for an infusion-ready drug product.

In some embodiments, the co-culture of the natural killer cell source, e.g., single unit of cord blood, with feeder cells yields from or from about 50×10⁸ to or to about 50×10¹² cells, e.g., MCB cells or infusion-ready drug product cells. In some embodiments, the expansion yields from or from about 50×10⁸ to or to about 25×10¹⁰, from or from about 10×10⁸ to or to about 1×10¹⁰, from or from about 50×10⁸ to or to about 75×10⁹, from or from about 50×10⁸ to or to about 50×10⁹, from or from about 50×10⁸ to or to about 25×10⁹, from or from about 50×10⁸ to or to about 1×10⁹, from or from about 50×10⁸ to or to about 75×10⁸, from or from about 75×10⁸ to or to about 50×10¹⁰, from or from about 75×10⁸ to or to about 25×10¹⁰, from or from about 75×10⁸ to or to about 1×10¹⁰, from or from about 75×10⁸ to or to about 75×10⁹, from or from about 75×10⁸ to or to about 50×10⁹, from or from about 75×10⁸ to or to about 25×10⁹, from or from about 75×10⁸ to or to about 1×10⁹, from or from about 1×10⁹ to or to about 50×10¹⁰, from or from about 1×10⁹ to or to about 25×10¹⁰, from or from about 1×10⁹ to or to about 1×10¹⁰, from or from about 1×10⁹ to or to about 75×10⁹, from or from about 1×10⁹ to or to about 50×10⁹, from or from about 1×10⁹ to or to about 25×10⁹, from or from about 25×10⁹ to or to about 50×10¹⁰, from or from about 25×10⁹ to or to about 25×10¹⁰, from or from about 25×10⁹ to or to about 1×10¹⁰, from or from about 25×10⁹ to or to about 75×10⁹, from or from about 25×10⁹ to or to about 50×10⁹, from or from about 50×10⁹ to or to about 50×10¹⁰, from or from about 50×10⁹ to or to about 25×10¹⁰, from or from about 50×10⁹ to or to about 1×10¹⁰, from or from about 50×10⁹ to or to about 75×10⁹, from or from about 75×10⁹ to or to about 50×10¹⁰, from or from about 75×10⁹ to or to about 25×10¹⁰, from or from about 75×10⁹ to or to about 1×10¹⁰, from or from about 1×10¹⁰ to or to about 50×10¹⁰, from or from about 1×10¹⁰ to or to about 25×10¹⁰, or from or from about 25×10¹⁰ to or to about 50×10¹⁰ cells, e.g., e.g., MCB cells or infusion-ready drug product cells.

In some embodiments, the expansion yields from or from about 60 to or to about 100 vials, each comprising from or from about 600 million to or to about 1 billion cells, e.g., MCB cells or infusion-ready drug product cells. In some embodiments, the expansion yields 80 or about 80 vials, each comprising or consisting of 800 million or about 800 million cells, e.g., MCB cells or infusion-ready drug product cells.

In some embodiments, the expansion yields from or from about a 100 to or to about a 500 fold increase in the number of cells, e.g., the number of MCB cells relative to the number of cells, e.g., NK cells, in the natural killer cell source. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of MCB cells relative to the number of cells, e.g., NK cells, in the natural killer cell source.

In some embodiments, the expansion yields from or from about a 100 to or to about a 70,000 fold increase in the number of cells, e.g., the number of MCB cells relative to the number of cells, e.g., NK cells, in the natural killer cell source. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of MCB cells relative to the number of cells, e.g., NK cells, in the natural killer cell source.

In some embodiments, the co-culture of the MCB cells and feeder cells yields from or from about 500 million to or to about 1.5 billion cells, e.g., NK cells suitable for use in an MCB and/or in an infusion-ready drug product. In some embodiments, the co-culture of the MCB cells and feeder cells yields from or from about 500 million to or to about 1.5 billion, from or from about 500 million to or to about 1.25 billion, from or from about 500 million to or to about 1 billion, from or from about 500 million to or to about 750 million, from or from about 750 million to or to about 1.5 billion, from or from about 500 million to or to about 1.25 billion, from or from about 750 million to or to about 1 billion, from or from about 1 billion to or to about 1.5 billion, from or from about 1 billion to or to about 1.25 billion, or from or from about 1.25 billion to or to about 1.5 billion cells, e.g., NK cells suitable for use in an MCB and/or an infusion-ready drug product.

In some embodiments, the co-culture of the MCB cells and feeder cells yields from or from about 50 to or to about 150 vials of cells, e.g., infusion-ready drug product cells, each comprising from or from about 750 million to or to about 1.25 billion cells, e.g., NK cells suitable for use in an MCB and/or an infusion-ready drug product. In some embodiments, the co-culture of the MCB cells and feeder cells yields 100 or about 100 vials, each comprising or consisting of 1 billion or about 1 billion cells, e.g., NK cells suitable for use in an MCB and/or an infusion-ready drug product.

In some embodiments, the expansion yields from or from about a 100 to or to about a 500 fold increase in the number of cells, e.g., the number of NK cells suitable for use in an MCB and/or an infusion-ready drug product relative to the number of starting MCB cells. In some embodiments, the expansion yields from or from about a 100 to or to about a 500, from or from about a 100 to or to about a 400, from or from about a 100 to or to about a 300, from or from about a 100 to or to about a 200, from or from about a 200 to or to about a 450, from or from about a 200 to or to about a 400, from or from about a 100 to or to about a 350, from or from about a 200 to or to about a 300, from or from about a 200 to or to about a 250, from or from about a 250 to or to about a 500, from or from about a 250 to or to about a 450, from or from about a 200 to or to about a 400, from or from about a 250 to or to about a 350, from or from about a 250 to or to about a 300, from or from about a 300 to or to about a 500, from or from about a 300 to or to about a 450, from or from about a 300 to or to about a 400, from or from about a 300 to or to about a 350, from or from about a 350 to or to about a 500, from or from about a 350 to or to about a 450, from or from about a 350 to or to about a 400 fold increase in the number of cells, e.g., the number of NK cells suitable for use in an MCB and/or an infusion-ready drug product relative to the number of starting MCB cells.

In some embodiments, the expansion yields from or from about a 100 to or to about a 70,000 fold increase in the number of cells, e.g., the number of NK cells suitable for use in an MCB and/or an infusion-ready drug product relative to the number of starting MCB cells. In some embodiments, the expansion yields at least a 10,000 fold, e.g., 15,000 fold, 20,000 fold, 25,000 fold, 30,000 fold, 35,000 fold, 40,000 fold, 45,000 fold, 50,000 fold, 55,000 fold, 60,000 fold, 65,000 fold, or 70,000 fold increase in the number of cells, e.g., the number of NK cells suitable for use in an MCB and/or an infusion-ready drug product relative to the number of starting MCB cells.

In embodiments where the cells are engineered during expansion and stimulation, as described herein, not all of the expanded and stimulated cells will necessarily be engineered successfully, e.g., transduced successfully, e.g., transduced successfully with a vector comprising a heterologous protein, e.g., a heterologous protein comprising a CAR and/or IL-15 as described herein. Thus, the methods described herein can further comprise sorting engineered cells, e.g., engineered cells described herein, away from non-engineered cells.

In some embodiments, the engineered cells, e.g., transduced cells, are sorted from the non-engineered cells, e.g., the non-transduced cells using a reagent specific to an antigen of the engineered cells, e.g., an antibody that targets an antigen of the engineered cells but not the non-engineered cells. In some embodiments, the antigen of the engineered cells is a component of a CAR, e.g., a CAR described herein.

Systems for antigen-based cell separation of cells are available commercially, e.g., the CliniMACS® sorting system (Miltenyi Biotec).

In some embodiments, the engineered cells, e.g., transduced cells, are sorted from the non-engineered cells, e.g., the non-transduced cells using flow cytometry.

In some embodiments, the sorted engineered cells are used as an MCB. In some embodiments, the sorted engineered cells are used as a component in an infusion-ready drug product.

In some embodiments, the engineered cells, e.g., transduced cells, are sorted from the non-engineered cells, e.g., the non-transduced cells using a microfluidic cell sorting method. Microfluidic cell sorting methods are described, for example, in Dalili et al., “A Review of Sorting, Separation and Isolation of Cells and Microbeads for Biomedical Applications: Microfluidic Approaches,” Analyst 144:87 (2019).

In some embodiments, from or from about 1% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein. In some embodiments, from or from about 1% to or to about 90%, from or from about 1% to or to about 80%, from or from about 1% to or to about 70%, from or from about 1% to or to about 60%, from or from about 1% to or to about 50%, from or from about 1% to or to about 40%, from or from about 1% to or to about 30%, from or from about 1% to or to about 20%, from or from about 1% to or to about 10%, from or from about 1% to or to about 5%, from or from about 5% to or to about 99%, from or from about 5% to or to about 90%, from or from about 5% to or to about 80%, from or from about 5% to or to about 70%, from or from about 5% to or to about 60%, from or from about 5% to or to about 50%, from or from about 5% to or to about 40%, from or from about 5% to or to about 30%, from or from about 5% to or to about 20%, from or from about 5% to or to about 10%, from or from about 10% to or to about 99%, from or from about 10% to or to about 90%, from or from about 10% to or to about 80%, from or from about 10% to or to about 70%, from or from about 10% to or to about 60%, from or from about 10% to or to about 50%, from or from about 10% to or to about 40%, from or from about 10% to or to about 30%, from or from about 10% to or to about 20%, from or from about 20% to or to about 99%, from or from about 20% to or to about 90%, from or from about 20% to or to about 80%, from or from about 20% to or to about 70%, from or from about 20% to or to about 60%, from or from about 20% to or to about 50%, from or from about 20% to or to about 40%, from or from about 20% to or to about 30%, from or from about 30% to or to about 99%, from or from about 30% to or to about 90%, from or from about 30% to or to about 80%, from or from about 30% to or to about 70%, from or from about 30% to or to about 60%, from or from about 30% to or to about 50%, from or from about 30% to or to about 40%, from or from about 40% to or to about 99%, from or from about 40% to or to about 90%, from or from about 40% to or to about 80%, from or from about 40% to or to about 70%, from or from about 40% to or to about 70%, from or from about 40% to or to about 60%, from or from about 40% to or to about 50%, from or from about 50% to or to about 99%, from or from about 50% to or to about 90%, from or from about 50% to or to about 80%, from or from about 50% to or to about 70%, from or from about 50% to or to about 60%, from or from about 60% to or to about 99%, from or from about 60% to or to about 90%, from or from about 60% to or to about 80%, from or from about 60% to or to about 70%, from or from about 70% to or to about 99%, from or from about 70% to or to about 90%, from or from about 70% to or to about 80%, from or from about 80% to or to about 99%, from or from about 80% to or to about 90%, or from or from about 90% to or to about 99% of the expanded and stimulated cells are engineered successfully, e.g., transduced successfully, e.g., transduced successfully with a vector comprising a heterologous protein, e.g., a heterologous protein comprising a CAR and/or IL-15 as described herein.

In some embodiments, frozen cells of a first or second MCB are thawed and cultured. In some embodiments, a single vial of frozen cells of the first or second MCB e.g., a single vial comprising 800 or about 800 million cells, e.g., first or second MCB cells, are thawed and cultured. In some embodiments, the frozen first or second MCB cells are cultured with additional feeder cells to produce cells suitable for use either as a second or third MCB or in an infusion-ready drug product. In some embodiments, the cells from the co-culture of the first or second MCB are harvested and frozen.

In some embodiments, the cells from the co-culture of the natural killer cell source, a first MCB, or a second MCB are harvested, and frozen in a cryopreservation composition, e.g., a cryopreservation composition described herein. In some embodiments, the cells are washed after harvesting. Thus, provided herein is a pharmaceutical composition comprising activated and stimulated NK cells, e.g., activated and stimulated NK cells produced by the methods described herein, e.g., harvested and washed activated and stimulated NK cells produced by the methods described herein and a cryopreservation composition, e.g., a cryopreservation composition described herein.

In some embodiments, the cells are mixed with a cryopreservation composition, e.g., as described herein, before freezing. In some embodiments, the cells are frozen in cryobags. In some embodiments, the cells are frozen in cryovials.

In some embodiments, the method further comprises isolating NK cells from the population of expanded and stimulated NK cells.

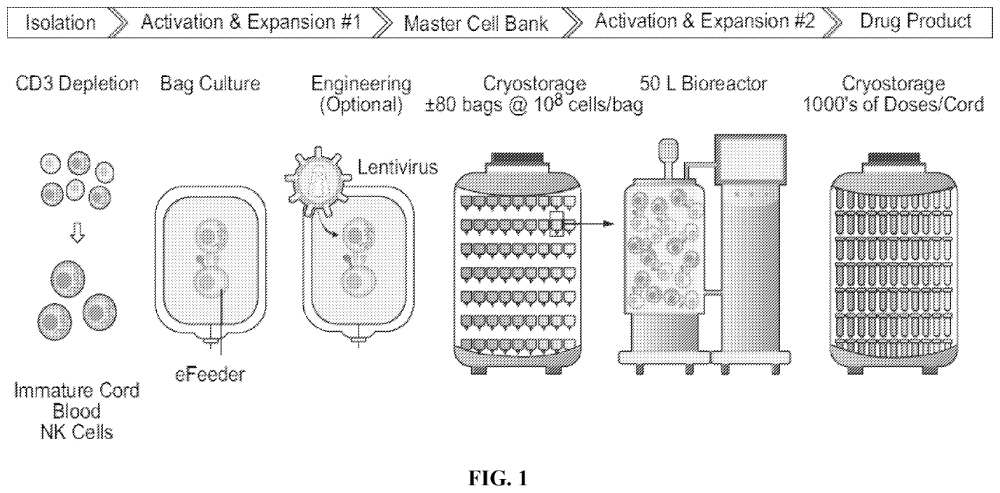

An exemplary process for expanding and stimulating NK cells is shown in FIG. 1.

### 5. Engineering

In some embodiments, the method further comprises engineering NK cell(s), e.g., to express a heterologous protein, e.g., a heterologous protein described herein, e.g., a heterologous protein comprising a CAR and/or IL-15.

In some embodiments, engineering the NK cell(s) to express a heterologous protein described herein comprises transforming or transducing, e.g., stably transforming or transducing the NK cells with a vector comprising a polynucleic acid encoding a heterologous protein described herein. Suitable vectors are described herein.

In some embodiments, engineering the NK cell(s) to express a heterologous protein described herein comprises introducing the heterologous protein via gene editing (e.g., zinc finger nuclease (ZFN) gene editing, ARCUS gene editing, CRISPR-Cas9 gene editing, or megaTAL gene editing) combined with adeno-associated virus (AAV) technology.

In some embodiments, the NK cell(s) are engineered to express a heterologous protein described herein, e.g., during or after culturing the composition in a medium comprising feeder cells. For example, in some cases, engineering (e.g., transduction) occurs during the expansion and stimulation process described herein, e.g., during co-culturing NK cell source(s) and feeder cell(s) as described herein, e.g., at day 1, 2, 3, 4, 5, 6, 7, 8, 9, 10, 11, 12, 13, 14, 15, 16, 17, or 18 of co-culturing.

In some embodiments, the method further comprises engineering NK cell(s), e.g., to express, over-express, knock-out, or knock-down gene(s) or gene product(s).

In some embodiments, the natural killer cells are not genetically engineered.

In some embodiments, the NK cell(s) are engineered (e.g., transduced) in a culture medium supplemented with a stimulating factor (e.g., as described herein). Such cytokines can be used to provide growth or survival signals to the NK cells during the engineering process or to increase transduction efficiency. In some embodiments, the stimulation factor(s) are cytokine(s). In some embodiments, the cytokine(s) are selected from the group consisting of IL-2, IL-12, IL-15, IL-18, IL-21, IL-23, IL-27, IFN-α, IFNβ, and combinations thereof.

In some embodiments, the cytokine is IL-21. IL-21 can be used, for example, at a final concentration of between 10 and 100 ng/mL, including, for example, at or at about 10, 15, 20, 25, 30, 34, 40, 45, 50, 55, 60, 70, 80, 90, or 100 ng/mL. In some embodiments, the cytokine is IL-2. In some embodiments, the cytokines are a combination of IL-2 and IL-21. In some embodiments, the cytokines are a combination of IL-2, IL-18, and IL-21.

In some embodiments, the stimulating factor is added to the culture medium at the time of engineering (e.g., transduction). In some embodiments, the stimulating factor is added to the culture medium after the time of engineered (e.g., transducing), e.g., from 1 to 48 hours after engineering, e.g., from 1 to 36, 1 to 24, 1 to 12, 12 to 28, 12 to 36, 12 to 24, 24 to 48, 24 to 36, or 36 to 48 hours after engineering. In some embodiments, the stimulating factor is added to the culture medium both at the time of transduction and after the time of engineering (e.g., from 1 to 48 hours after transduction).

In some embodiments, the culture is supplemented with the stimulating factor after culturing in a medium comprising feeder cells. Thus, in some cases, the culture medium will contain feeder cells at the time of engineering (e.g., transduction). In some cases, the feeder cells are removed from the culture prior to supplementation with the stimulating factor or engineering. In some cases, the feeder cells are not removed from the culture prior to supplementation with the stimulating factor or engineering. In some cases, no additional feeder cells are added to the culture during engineering, whether or not any residual feeder cells are removed. In some cases, both additional feeder cells and a stimulating factor are added to the culture during engineering. In some cases, additional feeder cells are not added to the culture during engineering but stimulating factors are added to the culture during engineering.

### E. Properties of Expanded and Stimulated NK Cells

After having been ex vivo expanded and stimulated, e.g., as described herein, the expanded and stimulated NK cell populations not only have a number/density (e.g., as described above) that could not occur naturally in the human body, but they also differ in their phenotypic characteristics, (e.g., gene expression and/or surface protein expression) with the starting source material or other naturally occurring populations of NK cells.

In some cases, the starting NK cell source is a sample derived from a single individual, e.g., a single cord blood unit that has not been ex vivo expanded. Therefore, in some cases, the expanded and stimulated NK cells share a common lineage, i.e., they all result from expansion of the starting NK cell source, and, therefore, share a genotype via clonal expansion of a population of cells that are, themselves, from a single organism. Yet, they could not occur naturally at the density achieved with ex vivo expansion and also differ in phenotypic characteristics from the starting NK cell source.

In some cases, the population of expanded and stimulated NK cells comprises at least 100 million expanded natural killer cells, e.g., 200 million, 250 million, 300 million, 400 million, 500 million, 600 million, 700 million, 750 million, 800 million, 900 million, 1 billion, 2 billion, 3 billion, 4 billion, 5 billion, 6 billion, 7 billion, 8 billion, 9 billion, 10 billion, 15 billion, 20 billion, 25 billion, 50 billion, 75 billion, 80 billion, 9-billion, 100 billion, 200 billion, 250 billion, 300 billion, 400 billion, 500 billion, 600 billion, 700 billion, 800 billion, 900 billion, 1 trillion, 2 trillion, 3 trillion, 4 trillion, 5 trillion, 6 trillion, 7 trillion, 8 trillion, 9 trillion, or 10 trillion expanded natural killer cells.

In some embodiments, the expanded and stimulated NK cells comprise at least 80%, e.g., at least 90%, at least 95%, at least 99%, or 100% CD56+CD3− cells. In some embodiments, the expanded and stimulated NK cells are not genetically engineered. In some embodiments, the expanded and stimulated NK cells do not comprise a CD16 transgene. In some embodiments, the expanded and stimulated NK cells do not express an exogenous CD16 protein.

The expanded and stimulated NK cells can be characterized, for example, by surface expression, e.g., of one or more of CD16, CD56, CD3, CD38, CD14, CD19, NKG2D, NKp46, NKp30, DNAM-1, and NKp44.

The surface protein expression levels stated herein, in some cases are achieved without positive selection on the particular surface protein referenced. For example, in some cases, the NK cell source, e.g., a single cord unit, comprises both the KIR B allele of the KIR receptor family and the 158 V/V variant of CD16 and is + enriched and CD3(+) depleted, e.g., by gating on CD56+CD3− expression, but no other surface protein expression selection is carried out during expansion and stimulation.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKG2D+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp46+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp30+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% DNAM-1+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% NKp44+ cells.

In some embodiments, the expanded and stimulated NK cells, e.g., from expansion and stimulation of a single cord blood unit, e.g., as described above, comprise at least 60%, e.g., at least 70%, at least 80%, at least 90% at least 95%, at least 99%, or 100% CD94+(KLRD1) cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD3+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD14+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD19+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CXCR+ cells.

In some embodiments, the expanded and stimulated NK cells, e.g., from expansion and stimulation of a single cord blood unit, e.g., as described above, comprises less than or equal to 20%, e.g., less than or equal to 10%, less than or equal to 5%, less than or equal to 1% or 0% CD122+(IL2RB) cells.

As described herein, the inventors have demonstrated that, surprisingly, the NK cells expanded and stimulated by the methods described herein express CD16 at high levels throughout the expansion and stimulation process, resulting in a cell population with high CD16 expression. The high expression of CD16 obviates the need for engineering the expanded cells to express CD16, which is important for initiating ADCC, and, therefore, a surprising and unexpected benefit of the expansion and stimulation methods described herein. Thus, in some embodiments, the expanded and stimulated NK cells, e.g., from expansion and stimulation of a single cord blood unit, e.g., as described above, comprise 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise 50% or more, e.g., 55%, 60%, 65%, 70%, 75%, 80%, 85%, 90%, or 95% CD16+NK cells.

In some embodiments, the percentage of expanded and stimulated NK cells, e.g., from expansion and stimulation of a single cord blood unit, e.g., as described above, expressing CD16 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKG2D is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp30 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing DNAM-1 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp44 is the same or higher than the percentage of natural killer cells in the seed cells from umbilical cord blood.

In some embodiments, the percentage of expanded and stimulated NK cells, e.g., from expansion and stimulation of a single cord blood unit, e.g., as described above, expressing NKp46 is the same or higher than the percentage of natural killer cells in the seed cells from umbilical cord blood.

As described herein, the inventors have also demonstrated that, surprisingly, the NK cells expanded and stimulated by the methods described herein express CD38 at low levels. CD38 is an effective target for certain cancer therapies (e.g., multiple myeloma and acute myeloid leukemia). See, e.g., Jiao et al., “CD38: Targeted Therapy in Multiple Myeloma and Therapeutic Potential for Solid Cancerrs,” Expert Opinion on Investigational Drugs 29(11):1295-1308 (2020). Yet, when an anti-CD38 antibody is administered with NK cells, because NK cells naturally express CD38, they are at risk for increased fratricide. The NK cells expanded and stimulated by the methods described herein, however, express low levels of CD38 and, therefore, overcome the anticipated fratricide. While other groups have resorted to engineering methods such as genome editing to reduce CD38 expression (see, e.g., Gurney et al., “CD38 Knockout Natural Killer Cells Expressing an Affinity Optimized CD38 Chimeric Antigen Receptor Successfully Target Acute Myeloid Leukemia with Reduced Effector Cell Fratricide,” Haematologica doi:10.3324/haematol.2020.271908 (2020), the NK cells expanded and stimulated by the methods described herein express low levels of CD38 without the need for genetic engineering, which provides a surprising and unexpected benefits, e.g., for treating CD38+ cancers with the NK cells expanded and stimulated as described herein, e.g., in combination with a CD38 antibody.

Thus, in some embodiments, the expanded and stimulated NK cells, e.g., from expansion and stimulation of a single cord blood unit, e.g., as described above, comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise less than or equal to 80% CD38+ cells, e.g., less than or equal to 75%, 70%, 65%, 60%, 55%, 50%, 45%, 40%, 35%, 30%, 25%, or 20% CD38+ cells, and 50% or more, e.g., 55%, 60%, 65%, 70%, 75%, 80%, 85%, 90%, or 95% CD16+NK cells.

In some embodiments, the expanded and stimulated NK cells, e.g., from expansion and stimulation of a single cord blood unit, e.g., as described above, comprises both the KIR B allele of the KIR receptor family and the 158 V/V variant of CD16 and comprise: i) 50% or more, e.g., 55%, 60%, 65%, 70%, 75%, 80%, 85%, 90%, or 95% CD16+NK cells; and/or ii) less than or equal to 80% CD38+ cells, e.g., less than or equal to 75%, 70%, 65%, 60%, 55%, 50%, 45%, 40%, 35%, 30%, 25%, or 20% CD38+ cells; and/or iii) at least 60%, e.g., at least 70%, at least 80%, at least 90% at least 95%, at least 99%, or 100% NKG2D+ cells; and/or iv) at least 60%, e.g., at least 70%, at least 80%, at least 90% at least 95%, at least 99%, or 100% NKp46+ cells; and/or v) at least 60%, e.g., at least 70%, at least 80%, at least 90% at least 95%, at least 99%, or 100% NKp30+ cells; and/or vi) at least 60%, e.g., at least 70%, at least 80%, at least 90% at least 95%, at least 99%, or 100% DNAM-1+ cells; and/or vii) at least 60%, e.g., at least 70%, at least 80%, at least 90% at least 95%, at least 99%, or 100% NKp44+ cells; and/or viii) at least 60%, e.g., at least 70%, at least 80%, at least 90% at least 95%, at least 99%, or 100% CD94+(KLRD1) cells; and/or ix) less than or equal to 20%, e.g., less than or equal to 10%, less than or equal to 5%, less than or equal to 1% or 0% CD3+ cells; and/or x) less than or equal to 20%, e.g., less than or equal to 10%, less than or equal to 5%, less than or equal to 1% or 0% CD14+ cells; and/or xi) less than or equal to 20%, e.g., less than or equal to 10%, less than or equal to 5%, less than or equal to 1% or 0% CD19+ cells; and/or xii) less than or equal to 20%, e.g., less than or equal to 10%, less than or equal to 5%, less than or equal to 1% or 0% CXCR+ cells; and/or xiii) less than or equal to 20%, e.g., less than or equal to 10%, less than or equal to 5%, less than or equal to 1% or 0% CD122+(IL2RB) cells.

In some embodiments, feeder cells do not persist in the expanded and stimulated NK cells, though, residual signature of the feeder cells may be detected, for example, by the presence of residual cells (e.g., by detecting cells with a particular surface protein expression) or residual nucleic acid and/or proteins that are expressed by the feeder cells.

For example, in some cases, the methods described herein include expanding and stimulating natural killer cells using engineered feeder cells, e.g., eHuT-78 feeder cells described above, which are engineered to express sequences that are not expressed by cells in the natural killer cell source, including the natural killer cells. For example, the engineered feeder cells can be engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof.

While these feeder cells may not persist in the expanded and stimulated NK cells, the expanded and stimulated NK cells may retain detectable residual amounts of cells, proteins, and/or nucleic acids from the feeder cells. Thus, their residual presence in the expanded and stimulated NK cells may be detected, for example, by detecting the cells themselves (e.g., by flow cytometry), proteins that they express, and/or nucleic acids that they express.

Thus, also described herein is a population of expanded and stimulated NK cells comprising residual feeder cells (live cells or dead cells) or residual feeder cell cellular impurities (e.g., residual feeder cell proteins or portions thereof, and/or genetic material such as a nucleic acid or portion thereof). In some cases, the expanded and stimulated NK cells comprise more than 0% and, but 0.3% or less residual feeder cells, e.g., eHuT-78 feeder cells.

In some cases, the expanded and stimulated NK cells comprise residual feeder cell nucleic acids, e.g., encoding residual 4-1BBL (UniProtKB P41273, SEQ ID NO: 1), membrane bound IL-21 (SEQ ID NO: 2), and/or mutant TNFalpha (SEQ ID NO: 3) or portion(s) thereof. In some cases, the membrane bound IL-21 comprises a CD8 transmembrane domain

In some cases, the expanded and stimulated NK cells comprise a % residual feeder cells of more than 0% and less than or equal to 0.2%, as measured, e.g., by the relative proportion of a feeder cell specific protein or nucleic acid sequence (that is, a protein or nucleic acid sequence not expressed by the natural killer cells) in the sample. For example, by qPCR, e.g., as described herein.

In some embodiments, the residual feeder cells are CD4(+) T cells. In some embodiments, the residual feeder cells are engineered CD4(+) T cells. In some embodiments, the residual feeder cell cells are engineered to express at least one gene selected from the group consisting of 4-1BBL (UniProtKB P41273, SEQ ID NO: 1), membrane bound IL-21 (SEQ ID NO: 2), and mutant TNFalpha (SEQ ID NO: 3) (“eHut-78 cells”), or variants thereof. Thus, in some cases, the feeder cell specific protein is 4-1BBL (UniProtKB P41273, SEQ ID NO: 1), membrane bound IL-21 (SEQ ID NO: 2), and/or mutant TNFalpha (SEQ ID NO: 3). And, therefore, the feeder cell specific nucleic acid is a nucleic acid encoding 4-1BBL (UniProtKB P41273, SEQ ID NO: 1), membrane bound IL-21 (SEQ ID NO: 2), and/or mutant TNFalpha (SEQ ID NO: 3), or portion thereof. In some cases, the membrane bound IL-21 comprises a CD8 transmembrane domain.

In some embodiments, the residual feeder cells are detected by the method described in.

A wide variety of different methods can be used to analyze and detect the presence of nucleic acids or protein gene products in a biological sample. As used herein, “detecting” can refer to a method used to discover, determine, or confirm the existence or presence of a compound and/or substance (e.g., a cell, a protein and/or a nucleic acid). In some embodiments, a detecting method can be used to detect a protein. In some embodiments, detecting can include chemiluminescence or fluorescence techniques. In some embodiments, detecting can include immunological-based methods (e.g., quantitative enzyme-linked immunosorbent assays (ELISA), Western blotting, or dot blotting) wherein antibodies are used to react specifically with entire proteins or specific epitopes of a protein. In some embodiments, detecting can include immunoprecipitation of the protein (Jungblut et al., J Biotechnol.31; 41(2-3):111-20 (1995); Franco et al., Eur J Morphol. 39(1):3-25 (2001)). In some embodiments, a detecting method can be used to detect a nucleic acid (e.g., DNA and/or RNA). In some embodiments, detecting can include Northern blot analysis, nuclease protection assays (NPA), in situ hybridization, or reverse transcription-polymerase chain reaction (RT-PCR) (Raj et al., Nat. Methods 5, 877-879 (2008); Jin et al., J Clin Lab Anal. 11(1):2-9 (1997); Ahmed, JEnviron Sci Health C Environ Carcinog Ecotoxicol Rev. 20(2):77-116 (2002)).

Thus, also described herein, are methods for detecting a population of expanded and stimulated NK cells, e.g., expanded and stimulated using the methods described herein, that have been co-cultured with engineered feeder cells, e.g., eHuT-78 feeder cells described herein.

### II. ANTI-CD19 CAR-NK

Provided herein are engineered cells, e.g., engineered natural killer cells, e.g., CAR-NK cells, e.g., anti-CD19 CAR-NK cells. In some embodiments, the CAR-NK cells are engineered to express IL-15.

In some embodiments, the natural killer cells are engineered, e.g., transduced, during expansion and stimulation, e.g., expansion and stimulation described herein. In some embodiments, the natural killer cells are engineered during expansion and stimulation, e.g., during production of a MCB, as described herein. In some embodiments, the natural killer cells are engineered during expansion and stimulation, e.g., during production of NK cells suitable for use in an injection-ready drug product and/or during production of a MCB, as described above. Thus, in some embodiments, the NK cell(s) are host cells and provided herein are NK host cell(s) expressing a heterogeneous protein, e.g., as described herein.

In some embodiments, the natural killer cells are engineered prior to expansion and stimulation. In some embodiments, the natural killer cells are engineered after expansion and stimulation.

In some embodiments, the NK cells are engineered by transducing with a vector. Suitable vectors are described herein, e.g., lentiviral vectors, e.g., a lentiviral vectors comprising a heterologous protein, e.g., as described herein. In some embodiments, the NK cells are transduced during production of a first MCB, as described herein.

In some embodiments, the NK cell(s) are transduced at a multiplicity of infection of from or from about 1 to or to about 40 viral particles per cell. In some embodiments, the NK cell(s) are transduced at a multiplicity of infection of or of about 1, of or of about 5, of or of about 10, of or of about 15, of or of about 20, of or of about 25, of or of about 30, of or of about 35, or of or of about 40 viral particles per cell.

### A. Chimeric Antigen Receptors

In some embodiments, the heterologous protein is a fusion protein, e.g., a fusion protein comprising a chimeric antigen receptor (CAR) is introduced into the NK cell, e.g., during the expansion and stimulation process.

In some embodiments, the CAR comprises one or more of: a signal sequence, an extracellular domain, a hinge, a transmembrane domain, and one or more intracellular signaling domain sequences. In some embodiments, the CAR further comprises a spacer sequence.

In some embodiments, the CAR comprises (from N- to C-terminal): a signal sequence, an extracellular domain, a hinge, a spacer, a transmembrane domain, a first signaling domain sequence, a second signaling domain sequence, and a third signaling domain sequence.

In some embodiments, the CAR comprises (from N- to C-terminal): a signal sequence, an extracellular domain, a hinge, a transmembrane domain, a first signaling domain sequence, a second signaling domain sequence, and a third signaling domain sequence.

The signal sequence can be cleaved from a mature CAR protein. Such cleavage can be mediated by a signal peptidase and can occur either during or after completion of translocation to generate the mature protein. Thus, in some embodiments, the CAR comprises (from N- to C-terminal): an extracellular domain, a hinge, a spacer, a transmembrane domain, a first signaling domain sequence, a second signaling domain sequence, and a third signaling domain sequence.

In some embodiments, the CAR comprises (from N- to C-terminal): an extracellular domain, a hinge, a transmembrane domain, a first signaling domain sequence, a second signaling domain sequence, and a third signaling domain sequence.

In some embodiments the extracellular domain comprises an antibody or antigen-binding portion thereof.

In some embodiments, one or more of the intracellular signaling domain sequence(s) is a CD28 intracellular signaling sequence. In some embodiments, the CD28 intracellular signaling sequence comprises or consists of SEQ ID NO: 5.

In some embodiments, one or more of the intracellular signaling domain sequence(s) is an OX40L signaling sequence. See, e.g., Matsumura et al., “Intracellular Signaling of gp34, the OX40 Ligand: Induction of c-jun and c-fos mRNA Expression Through gp34 upon Binding of Its Receptor, OX40,” J. Immunol 163:3007-11 (1999), which is hereby incorporated by reference in its entirety. In some embodiments, the OX40L signaling sequence comprises or consists of SEQ ID NO: 8, SEQ ID NO: 9, or SEQ ID NO: 10.

In some embodiments, one or more of the intracellular signaling sequence(s) is a CD3ζ intracellular signaling domain sequence. In some embodiments, the CD3ζ intracellular signaling sequence comprises of consists of SEQ ID NO: 13.

In some embodiments, the CAR comprises a CD28 intracellular signaling sequence (SEQ ID NO: 5), an OX40L intracellular signaling sequence (SEQ ID NO: 8, SEQ ID NO: 9, or SEQ ID NO: 10), and a CD3ζ intracellular signaling sequence (SEQ ID NO: 13).

In some embodiments, the CAR comprises an intracellular signaling domain comprising or consisting of SEQ ID NO: 25.

In some embodiments, the CAR does not comprise an OX40L intracellular signaling domain sequence.

In some embodiments, the CAR comprises a CD28 intracellular signaling sequence (SEQ ID NO: 5), and a CD3ζ intracellular signaling sequence (SEQ ID NO: 13), but not an OX40L intracellular signaling domain sequence.

In some embodiments, the signal sequence is a CD8α signal sequence. In some embodiments, the signal sequence comprises or consists of SEQ ID NO: 27.

In some embodiments, the extracellular domain comprises a single-chain variable fragment (scFv). In some embodiments, the extracellular domain comprises an anti-CD19 antibody or antigen binding fragment thereof. In some embodiments, the extracellular domain comprises an anti-CD19 scFv. In some embodiments, the anti-CD19 scFv comprises or consists of SEQ ID NO: 30.

In some embodiments, the hinge comprises or consists of an IgG1 hinge. In some embodiments, the IgG1 hinge comprises or consists of SEQ ID NO: 32.

In some embodiments, the spacer comprises or consists of an IgG1 CH2 and/or CH3 sequence or variant thereof, or combinations thereof. In some embodiments, the spacer comprises or consists of SEQ ID NO: 34. In some embodiments, the spacer comprises or consists of SEQ ID NO: 36.

In some embodiments, the transmembrane domain is a CD28 transmembrane domain. In some embodiments, the CD28 transmembrane domain comprises of consists of SEQ ID NO: 38.

In some embodiments, the transmembrane domain is a CD8 transmembrane domain. In some embodiments, the CD8 transmembrane domain comprises or consists of SEQ ID NO: 40.

In some embodiments, the fusion protein comprises or consists of SEQ ID NO: 42, SEQ ID NO: 46, SEQ ID NO: 50, or SEQ ID NO: 54. In some embodiments, the fusion protein comprises or consists of the amino sequence set forth in SEQ ID NO: 69, SEQ ID NO: 70, or SEQ ID NO: 71.

### B. IL-15

In some embodiments, the NK cell is engineered to express IL-15, e.g., human IL-15 (UniProtKB #P40933; NCBI Gene ID #3600), e.g., soluble human IL-15 or an ortholog thereof, or a variant of any of the foregoing. In some embodiments, the IL-15 is expressed as part of a fusion protein further comprising a cleavage site. In some embodiments, the IL-15 is expressed as part of a polyprotein comprising a self-cleaving peptide such as a T2A ribosomal skip sequence site. See, e.g., Radcliffe & Mitrophanous, “Multiple Gene Products from a Single Vector: ‘Self-Cleaving’ 2A Peptides,” Gene Therapy 11:1673-4 (2004); see also Liu et al., “Systematic Comparison of 2A Peptides for Cloning Multi-Genes in a Polycistronic Vector,” Scientific Reports 7(1):2193 (2017).

In some embodiments, the IL-15 comprises or consists of SEQ ID NO: 22.

In some embodiments, the self-cleaving peptide is a 2A self-cleaving peptide. In some embodiments, the self-cleaving peptide is a T2A, P2A, E2A, or F2A self-cleaving peptide. In some embodiments, the self-cleaving peptide comprises SEQ ID NO: 16. In some embodiments, the self-cleaving peptide comprises or consists of SEQ ID NO: 16, SEQ ID NO: 17, SEQ ID NO: 19, SEQ ID NO: 20, or SEQ ID NO: 21.

In some embodiments, the T2A cleavage site comprises or consists of SEQ ID NO: 17.

In some embodiments, the IL-15 is expressed as part of a fusion protein comprising a CAR, e.g., a CAR described herein.

In some embodiments, the fusion protein comprises (oriented from N-terminally to C-terminally): a CAR comprising, a cleavage site, and IL-15.

In some embodiments, the fusion protein comprises SEQ ID NO: 26.

In some embodiments, the fusion protein comprises or consists of SEQ ID NO: 44, SEQ ID NO: 48, SEQ ID NO: 52, or SEQ ID NO: 56.

### C. Inhibitory Receptors

In some embodiments, the NK cell is engineered to alter, e.g., reduce, expression of one or more inhibitor receptor genes.

In some embodiments, the inhibitory receptor gene is a HLA-specific inhibitory receptor. In some embodiments, the inhibitory receptor gene is a non-HLA-specific inhibitory receptor.

In some embodiments, the inhibitor receptor gene is selected from the group consisting of KIR, CD94/NKG2A, LILRB1, PD-1, IRp60, Siglec-7, LAIR-1, and combinations thereof.

### D. Polynucleic Acids, Vectors, and Host Cells

Also provided herein are polynucleic acids encoding the fusion protein(s) or portions thereof, e.g., the polynucleotide sequences encoding the polypeptides described herein, as shown in the Table of sequences provided herein

Also provided herein are vector(s) comprising the polynucleic acids, and cells, e.g., NK cells, comprising the vector(s).

In some embodiments, the vector is a lentivirus vector. See, e.g., Milone et al., “Clinical Use of Lentiviral Vectors,” Leukemia 32:1529-41 (2018). In some embodiments, the vector is a retrovirus vector. In some embodiments, the vector is a gamma retroviral vector. In some embodiments, the vector is a non-viral vector, e.g., a piggyback non-viral vector (PB transposon, see, e.g., Wu et al., “piggyback is a Flexible and Highly Active Transposon as Compared to Sleeping Beauty, Tol2, and Mosi in Mammalian Cells,” PNAS 103(41):15008-13 (2006)), a sleeping beauty non-viral vector (SB transposon, see, e.g., Hudecek et al., “Going Non-Viral: the Sleeping Beauty Transposon System Breaks on Through to the Clinical Side,” Critical Reviews in Biochemistry and Molecular Biology 52(4):355-380 (2017)), or an mRNA vector.

### III. CRYOPRESERVATION

### A. Cryopreservation Compositions

Provided herein are cryopreservation compositions, e.g., cryopreservation compositions suitable for intravenous administration, e.g., intravenous administration of NK cells, e.g., the NK cells described herein. In some embodiments, a pharmaceutical composition comprises the cryopreservation composition and cells, e.g., the NK cells described herein.

### 1. Albumin

In some embodiments, the cryopreservation composition comprises albumin protein, e.g., human albumin protein (UniProtKB Accession P0278, SEQ ID NO: 68) or variant thereof. In some embodiments, the cryopreservation composition comprises an ortholog of an albumin protein, e.g., human albumin protein, or variant thereof. In some embodiments, the cryopreservation composition comprises a biologically active portion of an albumin protein, e.g., human albumin, or variant thereof.

In some embodiments, the albumin, e.g., human albumin, is provided as a solution, also referred to herein as an albumin solution or a human albumin solution. Thus, in some embodiments, the cryopreservation composition is or comprises an albumin solution, e.g., a human albumin solution. In some embodiments, the albumin solution is a serum-free albumin solution.

In some embodiments, the albumin solution is suitable for intravenous use.

In some embodiments, the albumin solution comprises from or from about 40 to or to about 200 g/L albumin. In some embodiments, the albumin solution comprises from or from about 40 to or to about 50 g/L albumin, e.g., human albumin. In some embodiments, the albumin solution comprises about 200 g/L albumin, e.g., human albumin. In some embodiments, the albumin solution comprises 200 g/L albumin, e.g., human albumin.

In some embodiments, the albumin solution comprises a protein composition, of which 95% or more is albumin protein, e.g., human albumin protein. In some embodiments, 96%, 97%, 98%, or 99% or more of the protein is albumin, e.g., human albumin.

In some embodiments, the albumin solution further comprises sodium. In some embodiments, the albumin solution comprises from or from about 100 to or to about 200 mmol sodium. In some embodiments, the albumin solution comprises from or from about 130 to or to about 160 mmol sodium.

In some embodiments, the albumin solution further comprises potassium. In some embodiments, the albumin solution comprises 3 mmol or less potassium. In some embodiments, the albumin solution further comprises 2 mmol or less potassium.

In some embodiments, the albumin solution further comprises one or more stabilizers. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the solution comprises less than 0.1 mmol of each of the one or more stabilizers per gram of protein in the solution. In some embodiments, the solution comprises from or from about 0.05 to or to about 0.1, e.g., from or from about 0.064 to or to about 0.096 mmol of each of the stabilizers per gram of protein in the solution. In some embodiments, the solution comprises less than 0.1 mmol of total stabilizer per gram of protein in the solution. In some embodiments, the solution comprises from or from about 0.05 to or to about 0.1, e.g., from or from about 0.064 to or to about 0.096 mmol of total stabilizer per gram of protein in the solution.

In some embodiments, the albumin solution consists of a protein composition, of which 95% or more is albumin protein, sodium, potassium, and one or more stabilizers selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan) in water.

In some embodiments, the cryopreservation composition comprises from or from about 10% v/v to or to about 50% v/v of an albumin solution, e.g., an albumin solution described herein. In some embodiments, the cryopreservation composition comprises from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of an albumin solution described herein. In some embodiments, the cryopreservation composition comprises about 10%, about 15%, about 20%, about 25%, about 30%, about 35%, about 40%, about 45%, or about 50% v/v of an albumin solution described herein. In some embodiments, the cryopreservation composition comprises 10%, 15%, 20%, 25%, 30%, 35%, 40%, 45%, or 50% v/v of an albumin solution described herein.

In some embodiments, the cryopreservation composition comprises from or from about 20 to or to about 100 g/L albumin, e.g., human albumin. In some embodiments, the cryopreservation composition comprises from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 100, from or from about 80 to or to about 90, or from or from about 90 to or to about 100 g/L albumin, e.g., human albumin.

In some embodiments, the cryopreservation composition comprises 20 g/L albumin, e.g., human albumin. In some embodiments, the cryopreservation composition comprises 40 g/L albumin, e.g., human albumin. In some embodiments, the cryopreservation composition comprises 70 g/L albumin, e.g., human albumin. In some embodiments, the cryopreservation composition comprises 100 g/L albumin, e.g., human albumin.

In some embodiments, the cryopreservation composition comprises about 20 g/L albumin, e.g., human albumin. In some embodiments, the cryopreservation composition comprises about 40 g/L albumin, e.g., human albumin. In some embodiments, the cryopreservation composition comprises about 70 g/L albumin, e.g., human albumin. In some embodiments, the cryopreservation composition comprises about 100 g/L albumin, e.g., human albumin.

In some embodiments, the cryopreservation composition further comprises a stabilizer, e.g., an albumin stabilizer. In some embodiments, the stabilizer(s) are selected from the group consisting of sodium caprylate, caprylic acid, (2S)-2-acetamido-3-(1H-indol-3-yl)propanoic acid (also referred to as acetyl tryptophan, N-Acetyl-L-tryptophan and Acetyl-L-tryptophan), 2-acetamido-3-(1H-indol-3-yl)propanoic acid (also referred to as N-acetyltryptophan, DL-Acetyltroptohan and N-Acetyl-DL-tryptophan). In some embodiments, the cryopreservation composition comprises less than 0.1 mmol of each of the one or more stabilizers per gram of protein, e.g., per gram of albumin protein, in the composition. In some embodiments, the cryopreservation composition comprises from or from about 0.05 to or to about 0.1, e.g., from or from about 0.064 to or to about 0.096 mmol of each of the stabilizers per gram of protein, e.g., per gram of albumin protein in the composition. In some embodiments, the cryopreservation composition comprises less than 0.1 mmol of total stabilizer per gram of protein, e.g., per gram of albumin protein in the cryopreservation composition. In some embodiments, the cryopreservation composition comprises from or from about 0.05 to or to about 0.1, e.g., from or from about 0.064 to or to about 0.096 mmol of total stabilizer per gram of protein, e.g., per gram of albumin protein, in the cryopreservation composition.

### 2. Dextran

In some embodiments, the cryopreservation composition comprises Dextran, or a derivative thereof.

Dextran is a polymer of anhydroglucose composed of approximately 95% α-D-(1-6) linkages (designated (C6H1005)n). Dextran fractions are supplied in molecular weights of from about 1,000 Daltons to about 2,000,000 Daltons. They are designated by number (Dextran X), e.g., Dextran 1, Dextran 10, Dextran 40, Dextran 70, and so on, where X corresponds to the mean molecular weight divided by 1,000 Daltons. So, for example, Dextran 40 has an average molecular weight of or about 40,000 Daltons.

In some embodiments, the average molecular weight of the dextran is from or from about 1,000 Daltons to or to about 2,000,000 Daltons. In some embodiments, the average molecular weight of the dextran is or is about 40,000 Daltons. In some embodiments, the average molecular weight of the dextran is or is about 70,000 Daltons.

In some embodiments, the dextran is selected from the group consisting of Dextran 40, Dextran 70, and combinations thereof. In some embodiments, the dextran is Dextran 40.

In some embodiments, the dextran, e.g., Dextran 40, is provided as a solution, also referred to herein as a dextran solution or a Dextran 40 solution. Thus, in some embodiments, the composition comprises a dextran solution, e.g., a Dextran 40 solution.

In some embodiments, the dextran solution is suitable for intravenous use.

In some embodiments, the dextran solution comprises about 5% to about 50% w/w dextran, e.g., Dextran 40. In some embodiments, the dextran solution comprises from or from about 5% to or to about 50%, from or from about 5% to or to about 45%, from or from about 5% to or to about 40%, from or from about 5% to or to about 35%, from or from about 5% to or to about 30%, from or from about 5% to or to about 25%, from or from about 5% to or to about 20%, from or from about 5% to or to about 15%, from or from about 5% to or to about 10%, from or from about 10% to or to about 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% w/w dextran, e.g., Dextran 40. In some embodiments, the dextran solution comprises 5%, 10, 1%, 20%, 25%, 30%, 35%, 40%, 45%, or 50% w/w dextran, e.g., Dextran 40. In some embodiments, the dextran solution comprises about 5%, about 10%, about 15%, about 20%, about 25%, about 30%, about 35%, about 40%, about 45%, or about 50% w/w dextran, e.g., Dextran 40.

In some embodiments, the dextran solution comprises from or from about 25 g/L to or to about 200 g/L dextran, e.g., Dextran 40. In some embodiments, the dextran solution comprises from or from about 35 to or to about 200, from or from about 25 to or to about 175, from or from about 25 to or to about 150, from or from about 25 to or to about 125, from or from about 25 to or to about 100, from or from about 25 to or to about 75, from or from about 25 to or to about 50, from or from about 50 to or to about 200, from or from about 50 to or to about 175, from or from about 50 to or to about 150, from or from about 50 to or to about 125, from or from about 50 to or to about 100, from or from about 50 to or to about 75, from or from about 75 to or to about 200, from or from about 75 to or to about 175, from or from about 75 to or to about 150, from or from about 75 to or to about 125, from or from about 75 to or to about 100, from or from about 100 to or to about 200, from or from about 100 to or to about 175, from or from about 100 to or to about 150, from or from about 100 to or to about 125, from or from about 125 to or to about 200, from or from about 125 to or to about 175, from or from about 125 to or to about 150, from or from about 150 to or to about 200, from or from about 150 to or to about 175, or from or from about 175 to or to about 200 g/L dextran e.g., Dextran 40. In some embodiments, the dextran solution comprises 25, 50, 75, 100, 125, 150, 175, or 200 g/L dextran, e.g., Dextran 40. In some embodiments, the dextran solution comprises 100 g/L dextran, e.g., Dextran 40. In some embodiments, the dextran solution comprises about 25, about 50, about 75, about 100, about 125, about 150, about 175, or about 200 g/L dextran, e.g., Dextran 40. In some embodiments, the dextran solution comprises about 100 g/L dextran, e.g., Dextran 40.

In some embodiments, the dextran solution further comprises glucose (also referred to as dextrose). In some embodiments, the dextran solution comprises from or from about 10 g/L to or to about 100 g/L glucose. In some embodiments, the dextran solution comprises from or from about 10 to or to about 100, from or from about 10 to or to about 90, from or from about 10 to or to about 80, from or from about 10 to or to about 70, from or from about 10 to or to about 60, from or from about 10 to or to about 50, from or from about 10 to or to about 40, from or from about 10 to or to about 30, from or from about 10 to or to about 20, from or from about 20 to or to about 100, from or from about 20 to or to about 90, from or from about 20 to or to about 80, from or from about 20 to or to about 70, from or from about 20 to or to about 60, from or from about 20 to or to about 50, from or from about 20 to or to about 40, from or from about 20 to or to about 30, from or from about 30 to or to about 100, from or from about 30 to or to about 90, from or from about 30 to or to about 80, from or from about 30 to or to about 70, from or from about 30 to or to about 60, from or from about 30 to or to about 50, from or from about 30 to or to about 40, from or from about 40 to or to about 100, from or from about 40 to or to about 90, from or from about 40 to or to about 80, from or from about 40 to or to about 70, from or from about 40 to or to about 60, from or from about 40 to or to about 50, from or from about 50 to or to about 100, from or from about 50 to or to about 90, from or from about 50 to or to about 80, from or from about 50 to or to about 70, from or from about 50 to or to about 60, from or from about 60 to or to about 100, from or from about 60 to or to about 90, from or from about 60 to or to about 80, from or from about 60 to or to about 70, from or from about 70 to or to about 100, from or from about 70 to or to about 90, from or from about 70 to or to about 80, from or from about 80 to or to about 90, from or from about 80 to or to about 100, from or from about 80 to or to about 90, or from or from about 90 to or to about 100 g/L glucose. In some embodiments, the dextran solution comprises 10, 20, 30, 40, 50, 60, 70, 80, 90, or 100 g/L glucose. In some embodiments, the dextran solution comprises 50 g/L glucose. In some embodiments, the dextran solution comprises about 10, about 20, about 30, about 40, about 50, about 60, about 70, about 80, about 90, or about about 100 g/L glucose. In some embodiments, the dextran solution comprises 50 g/L glucose.

In some embodiments, the dextran solution consists of dextran, e.g., Dextran 40, and glucose in water.

In some embodiments, the cryopreservation composition comprises from or from about 10% v/v to or to about 50% v/v of a dextran solution described herein. In some embodiments, the cryopreservation composition comprises from or from about 10% to 50%, from or from about 10% to or to about 45%, from or from about 10% to or to about 40%, from or from about 10% to or to about 35%, from or from about 10% to or to about 30%, from or from about 10% to or to about 25%, from or from about 10% to or to about 20%, from or from about 10% to or to about 15%, from or from about 15% to or to about 50%, from or from about 15% to or to about 45%, from or from about 15% to or to about 40%, from or from about 15% to or to about 35%, from or from about 15% to or to about 30%, from or from about 15% to or to about 25%, from or from about 15% to or to about 20%, from or from about 20% to or to about 50%, from or from about 20% to or to about 45%, from or from about 20% to or to about 40%, from or from about 20% to or to about 35%, from or from about 20% to or to about 30%, from or from about 20% to or to about 25%, from or from about 25% to or to about 50%, from or from about 25% to or to about 45%, from or from about 25% to or to about 40%, from or from about 25% to or to about 35%, from or from about 25% to or to about 30%, from or from about 30% to or to about 50%, from or from about 30% to or to about 45%, from or from about 30% to or to about 40%, from or from about 30% to or to about 35%, from or from about 35% to or to about 50%, from or from about 35% to or to about 45%, from or from about 35% to or to about 40%, from or from about 40% to or to about 50%, from or from about 40% to or to about 45%, or from or from about 45% to or to about 50% v/v of a dextran solution, e.g., a dextran solution described herein. In some embodiments, the cryopreservation composition comprises 10%, 15%, 20%, 25%, 30%, 35%, 40%, 45%, or 50% v/v of a dextran solution, e.g., a dextran solution described herein. In some embodiments, the cryopreservation composition comprises about 10%, about 15%, about 20%, about 25%, about 30%, about 35%, about 40%, about 45%, or about 50% v/v of a dextran solution, e.g., a dextran solution described herein.

In some embodiments, the cryopreservation composition comprises from or from about 10 to or to about about 50 g/L dextran, e.g., Dextran 40. In some embodiments, the cryopreservation composition comprises from or from about 10 to or to about 50, from or from about 10 to or to about 45, from or from about 10 to or to about 40, from or from about 10 to or to about 35, from or from about 10 to or to about 30, from or from about 10 to or to about 25, from or from about 10 to or to about 20, from or from about 10 to or to about 15, from or from about 15 to or to about 50, from or from about 15 to or to about 45, from or from about 15 to or to about 40, from or from about 15 to or to about 35, from or from about 15 to or to about 30, from or from about 15 to or to about 25, from or from about 15 to or to about 20, from or from about 20 to or to about 50, from or from about 20 to or to about 45, from or from about 20 to or to about 40, from or from about 20 to or to about 30, from or from about 20 to or to about 25, from or from about 25 to or to about 50, from or from about 25 to or to about 45, from or from about 25 to or to about 40, from or from about 25 to or to about 35, from or from about 25 to or to about 30, from or from about 30 to or to about 50, from or from about 30 to or to about 45, from or from about 30 to or to about 40, from or from about 30 to or to about 35, from or from about 35 to or to about 50, from or from about 35 to or to about 45, from or from about 35 to or to about 40, from or from about 40 to or to about 50, from or from about 40 to or to about 45, or from or from about 45 to or to about 50 g/L dextran, e.g., Dextran 40. In some embodiments, the cryopreservation composition comprises 10, 15, 20, 25, 30, 30, 35, 40, 45, or 50 g/L dextran, e.g., Dextran 40. In some embodiments, the cryopreservation composition comprises about 10, about 15, about 20, about 25, about 30, about 30, about 35, about 40, about 45, or about 50 g/L dextran, e.g., Dextran 40.

### 3. Glucose

In some embodiments, the cryopreservation composition comprises glucose.

In some embodiments, as described above, the cryopreservation composition comprises a Dextran solution comprising glucose.

In some embodiments, the cryopreservation composition comprises a Dextran solution that does not comprise glucose. In some embodiments, e.g., when the Dextran solution does not comprise glucose, glucose is added separately to the cryopreservation composition.

In some embodiments, the cryopreservation composition comprises from or from about 5 to or to about 25 g/L glucose. In some embodiments, the cryopreservation composition comprises from or from about 5 to or to about 25, from or from about 5 to or to about 20, from or from about 5 to or to about 15, from or from about 5 to or to about 10, from or from about 10 to or to about 25, from or from about 10 to or to about 20, from or from about 10 to or to about 15, from or from about 15 to or to about 25, from or from about 15 to or to about 20, or from or from about 20 to or to about 25 g/L glucose. In some embodiments, the cryopreservation composition comprises 5, 7.5, 10, 12.5, 15, 17.5, 20, 22.5, or 25 g/L glucose. In some embodiments, the cryopreservation composition comprises 12.5 g/L glucose. In some embodiments, the cryopreservation composition comprises about 5, about 7.5, about 10, about 12.5, about 15, about 17.5, about 20, about 22.5, or about 25 g/L glucose. In some embodiments, the cryopreservation composition comprises about 12.5 g/L glucose.

In some embodiments, the cryopreservation composition comprises less than 2.75% w/v glucose. In some embodiments, the cryopreservation composition comprises less than 27.5 g/L glucose. In some embodiments, the cryopreservation composition comprises less than 2% w/v glucose. In some embodiments, the cryopreservation composition comprises less than 1.5% w/v glucose. In some embodiments, the cryopreservation composition comprises about 1.25% w/v or less glucose.

### 4. Dimethyl Sulfoxide

In some embodiments, the cryopreservation composition comprises dimethyl sulfoxide (DMSO, also referred to as methyl sulfoxide and methylsulfinylmethane).

In some embodiments, the DMSO is provided as a solution, also referred to herein as a DMSO solution. Thus, in some embodiments, the cryopreservation composition comprises a DMSO solution.

In some embodiments, the DMSO solution is suitable for intravenous use.

In some embodiments, the DMSO solution comprises 1.1 g/mL DMSO. In some embodiments, the DMSO solution comprises about 1.1 g/mL DMSO.

In some embodiments, the cryopreservation composition comprises from or from about 1% to or to about 10% v/v of the DMSO solution. In some embodiments, the cryopreservation composition comprises from or from about 1% to or to about 10%, from or from about 1% to or to about 9%, from or from about 1% to or to about 8%, from or from about 1% to or to about 7%, from or from about 1% to or to about 6%, from or from about 1% to or to about 5%, from or from about 1% to or to about 4%, from or from about 1% to or to about 3%, from or from about 1% to or to about 2%, from or from about 2% to or to about 10%, from or from about 2% to or to about 9%, from or from about 8%, from or from about 2% to or to about 7%, from or from about 2% to or to about 6%, from or from about 2% to or to about 5%, from or from about 2% to or to about 4%, from or from about 2% to or to about 3%, from or from about 3% to or to about 10%, from or from about 3% to or to about 9%, from or from about 3% to or to about 8%, from or from about 3% to or to about 7%, from or from about 3% to or to about 6%, from or from about 3% to or to about 5%, from or from about 3% to or to about 4%, from or from about 4% to or to about 10%, from or from about 4% to or to about 9%, from or from about 4% to or to about 8%, from or from about 4% to or to about 7%, from or from about 4% to or to about 6%, from or from about 4% to or to about 5%, from or from about 5% to or to about 10%, from or from about 5% to or to about 9%, from or from about 5% to or to about 8%, from or from about 5% to or to about 7%, from or from about 5% to or to about 6%, from or from about 6% to or to about 10%, from or from about 6% to or to about 9%, from or from about 6% to or to about 8%, from or from about 6% to or to about 7%, from or from about 7% to or to about 10%, from or from about 7% to or to about 9%, from or from about 7% to or to about 8%, from or from about 8% to or to about 10%, from or from about 8% to or to about 9%, or from or from about 9% to or to about 10% v/v of the DMSO solution. In some embodiments, the cryopreservation composition comprises 1%, 2%, 3%, 4%, 5%, 6%, 7%, 8%, 9%, or 10% v/v of the DMSO solution. In some embodiments, the cryopreservation composition comprises 5% of the DMSO solution. In some embodiments, the cryopreservation composition comprises about 1%, about 2%, about 3%, about 4%, about 5%, about 6%, about 7%, about 8%, about 9%, or about 10% v/v of the DMSO solution. In some embodiments, the cryopreservation composition comprises about 5% of the DMSO solution.

In some embodiments, the cryopreservation composition comprises from or from about 11 to or to about 110 g/L DMSO. In some embodiments, from or from about the cryopreservation composition comprises from or from about 11 to or to about 110, from or from about 11 to or to about 99, from or from about 11 to or to about 88, from or from about 11 to or to about 77, from or from about 11 to or to about 66, from or from about 11 to or to about 55, from or from about 11 to or to about 44, from or from about 11 to or to about 33, from or from about 11 to or to about 22, from or from about 22 to or to about 110, from or from about 22 to or to about 99, from or from about 22 to or to about 88, from or from about 22 to or to about 77, from or from about 22 to or to about 77, from or from about 22 to or to about 66, from or from about 22 to or to about 55, from or from about 22 to or to about 44, from or from about 22 to or to about 33, from or from about 33 to or to about 110, from or from about 33 to or to about 99, from or from about 33 to or to about 88, from or from about 33 to or to about 77, from or from about 33 to or to about 66, from or from about 33 to or to about 55, from or from about 33 to or to about 44, from or from about 44 to or to about 110, from or from about 44 to or to about 99, from or from about 44 to or to about 88, from or from about 44 to or to about 77, from or from about 44 to or to about 66, from or from about 44 to or to about 55, from or from about 55 to or to about 110, from or from about 55 to or to about 99, from or from about 55 to or to about 88, from or from about 55 to or to about 77, from or from about 55 to or to about 66, from or from about 66 to or to about 110, from or from about 66 to or to about 99, from or from about 66 to or to about 88, from or from about 66 to or to about 77, from or from about 77 to or to about 119, from or from about 77 to or to about 88, from or from about 88 to or to about 110, from or from about 88 to or to about 99, or from or from about 99 to or to about 110 g/L DMSO. In some embodiments, the cryopreservation composition comprises 11, 22, 33, 44, 55, 66, 77, 88, 99, or 110 g/L DMSO. In some embodiments, the cryopreservation composition comprises 55 g/L DMSO. In some embodiments, the cryopreservation composition comprises about 11, about 22, about 33, about 44, about 55, about 66, about 77, about 88, about 99, or about 110 g/L DMSO. In some embodiments, the cryopreservation composition comprises about 55 g/L DMSO.

### 5. Buffers

In some embodiments, the cryopreservation composition comprises a buffer solution, e.g., a buffer solution suitable for intravenous administration.

Buffer solutions include, but are not limited to, phosphate buffered saline (PBS), Ringer's Solution, Tyrode's buffer, Hank's balanced salt solution, Earle's Balanced Salt Solution, saline, and Tris.

In some embodiments, the buffer solution is phosphate buffered saline (PBS).

### 6. Exemplary Cryopreservation Compositions

In some embodiments, the cryopreservation composition comprises or consists of: 1) albumin, e.g., human albumin, 2) dextran, e.g., Dextran 40, 3) DMSO, and 4) a buffer solution. In some embodiments, the cryopreservation composition further comprises glucose. In some embodiments, the cryopreservation composition consists of 1) albumin, e.g., human albumin, 2) dextran, e.g., Dextran 40, 3) glucose, 4) DMSO, and 5) a buffer solution.

In some embodiments, the cryopreservation composition comprises: 1) an albumin solution described herein, 2) a dextran solution described herein, 3) a DMSO solution described herein, and 4) a buffer solution.

In some embodiments, the cryopreservation composition consists of: 1) an albumin solution described herein, 2) a dextran solution described herein, 3) a DMSO solution described herein, and 4) a buffer solution.

In some embodiments, the cryopreservation composition does not comprise a cell culture medium.

In one embodiment, the cryopreservation composition comprises or comprises about 40 mg/mL human albumin, 25 mg/mL Dextran 40, 12.5 mg/mL glucose, and 55 mg/mL DMSO.

In one embodiment, the cryopreservation composition comprises or comprises about or consists of or consists of about 40 mg/mL human albumin, 25 mg/mL Dextran 40, 12.5 mg/mL glucose, 55 mg/mL DMSO, and 0.5 mL/mL 100% phosphate buffered saline (PBS) in water.

In one embodiment, the cyopreservation composition comprises or comprises about 32 mg/mL human albumin, 25 mg/mL Dextran 40, 12.5 mg/mC glucose, and 55 mg/mE DMO. 03091 In one embodiment, the cryopreservation composition comprises or comprises about or consists ofor consists of about 32 mg/m human albumin, 25 mg/mE Dextran 40, 12.5 mg/mE glucose, 55 mg/mL DMuO, and 0.54 mE/mE500/L cphosphate buffered saline (PBS) in water.

Exemplary Cryopreservation Compositions are shown in Table 4.

TABLE 4
Exemplary Cryopreservation Compositions

| | Concentration | Exemplary | Exemplary Range v/v % |
|---|---|---|---|
| Excipient | Range of | Solution | in Cryopreservation |
| Solution | Solution | Concentration | Composition |
| Albumin | 40-200 g/L albumin in water | 200 g/L albumin | 10%-50% |
| Solution | | | |
| Dextran 40 | 25-200 g/L Dextran 40; and | 100 g/L Dextran 40; | 10%-50% |
| Solution | 0-100 g/L glucose; in water | 50 g/L glucose | |
| DMSO | 11-110 g/L DMSO | 1,100 g/L DMSO | 1%-10% |
| | in water | | |
| Buffer | to volume | to volume | to volume |

TABLE 5
Exemplary Cryopreservation Composition #1

| | | Exemplary v/v % | Final Concentration | |
|---|---|---|---|---|
| Excipient | Solution | in Cryopreservation | in Cryopreservation | |
| Solution | Composition | Composition #1 | Composition #1 | |
| Albumin | 200 g/L albumin in water | 20% | 40 mg/mL albumin | |
| Solution | | | | |
| Dextran 40 | 100 g/L Dextran 40; and | 25% | 25 mg/mL Dextran 40; | |
| Solution | 50 g/L glucose; in water | | 12.5 mg/mL glucose | |
| DMSO | 100% DMSO (1,100 g/L) | 5% | 55 | mg/mL |
| Buffer | 100% Phosphate Buffered | 50% | 0.5 | mL/mL |
| | Saline (PBS) | | | |

TABLE 6
Exemplary Cryopreservation Composition #2

| | | Exemplary v/v % | Final Concentration | |
|---|---|---|---|---|
| Excipient | Solution | in Cryopreservation | in Cryopreservation | |
| Solution | Composition | Composition #2 | Composition #2 | |
| Albumin | 200 g/L albumin in water | 16% | 32 mg/mL albumin | |
| Solution | | | | |
| Dextran 40 | 100 g/L Dextran 40; and | 25% | 25 mg/mL Dextran 40; | |
| Solution | 50 g/L glucose; in water | | 12.5 mg/mL glucose | |
| DMSO | 100% DMSO (1,100 g/L) | 5% | 55 | mg/mL |
| Buffer | 100% Phosphate Buffered | 54% | 0.54 | mL/mL |
| | Saline (PBS) | | | |

### B. Methods of Cryopreserving

The cryopreservation compositions described herein can be used for cryopreserving cell(s), e.g., therapeutic cells, e.g., natural killer (NK) cell(s), e.g., the NK cell(s) described herein. In some embodiments, the cell(s) are an animal cell(s). In some embodiments, the cell(s) are human cell(s). In some embodiments, the cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof. In some embodiments, the immune cell(s) are natural killer (NK) cells. In some embodiments, the natural killer cell(s) are expanded and stimulated by a method described herein. In some embodiments, the NK cell(s) are CAR-NK cell(s), for example CAR-NK cell(s) described herein.

In some embodiments, cryopreserving the cell(s) comprises: mixing the cell(s) with a cryopreservation composition or components thereof described herein to produce a composition, e.g., a pharmaceutical composition; and freezing the mixture.

In some embodiments, cryopreserving the cell(s) comprises: mixing a composition comprising the cell(s) with a cryopreservation composition or components thereof described herein to produce a composition, e.g., a pharmaceutical composition; and freezing the mixture. In some embodiments, the composition comprising the cell(s) comprises: the cell(s) and a buffer. Suitable buffers are described herein.

In some embodiments, cryopreserving the cell(s) comprises: mixing a composition comprising the cell(s) and a buffer, e.g., PBS, with a composition comprising albumin, Dextran, and DMSO, e.g., as described herein; and freezing the mixture.

In some embodiments, cryopreserving the cell(s) comprises: mixing a composition comprising the cell(s) and a buffer, e.g., PBS 1:1 with a composition comprising 40 mg/mL albumin, e.g., human albumin, 25 mg/mL Dextran, e.g., Dextran 40, 12.5 mg/mL glucose and 55 mg/mL DMSO.

In some embodiments, the composition comprising the cell(s) and the buffer, e.g., PBS, comprises from or from about 2×10⁷ to or to about 2×10⁹ cells/mL. In some embodiments, the composition comprising the cell(s) and the buffer, e.g., PBS, comprises 2×10⁸ cells/mL. In some embodiments, the composition comprising the cell(s) and the buffer, e.g., PBS, comprising about 2×10⁸ cells/mL.

In some embodiments, cryopreserving the cell(s) comprising mixing: the cell(s), a buffer, e.g., PBS, albumin, e.g., human albumin, Dextran, e.g., Dextran 40, and DMSO; and freezing the mixture.

In some embodiments, the mixture comprises from or from about 1×10⁷ to or to about 1×10⁹ cells/mL. In some embodiments, the mixture comprises 1×10⁸ cells/mL. In some embodiments, the mixture comprises about 1×10⁸ cells/mL.

Suitable ranges for albumin, Dextran, and DMSO are set forth above.

In some embodiments, the composition is frozen at or below −135° C.

In some embodiments, the composition is frozen at a controlled rate.

### IV. PHARMACEUTICAL COMPOSITIONS

Provided herein are pharmaceutical compositions comprising the natural killer cells described herein and dosage units of the pharmaceutical compositions described herein.

In some cases, the dosage unit comprises between 100 million and 1.5 billion cells, e.g., 100 million, 200 million, 300 million, 400 million, 500 million, 600 million, 700 million, 800 million, 900 million, 1 billion, 1.1 billion, 1.2 billion, 1.3 billion, 1.4 billion, or 1.5 billion.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

In some embodiments, the pharmaceutical composition comprises: a) natural killer cell(s) described herein; and b) a cryopreservation composition.

Suitable cryopreservation compositions are described herein.

In some embodiments, the composition is frozen. In some embodiments, the composition has been frozen for at least three months, e.g., at least six months, at least nine months, at least 12 months, at least 15 months, at least 18 months, at least 24 months, or at least 36 months. In some embodiments, at least 60%, e.g., at least 70%, at least 80%, at least 90% at least 95%, at least 99%, or 100% of the natural killer cells are viable after being thawed.

In some embodiments, the pharmaceutical composition comprises: a) a cryopreservation composition described herein; and b) therapeutic cell(s). In some embodiments, the therapeutic cell(s) are animal cell(s). In some embodiments, the therapeutic cell(s) are human cell(s). In some embodiments, the therapeutic cell(s) are immune cell(s). In some embodiments, the immune cell(s) are selected from basophils, eosinophils, neutrophils, mast cells, monocytes, macrophages, neutrophils, dendritic cells, natural killer cells, B cells, T cells, and combinations thereof. In some embodiments, the immune cell(s) are natural killer (NK) cells. In some embodiments, the natural killer cell(s) are expanded and stimulated by a method described herein, e.g., the CAR-NKs described herein.

In some embodiments, the pharmaceutical composition further comprises: c) a buffer solution. Suitable buffer solutions are described herein, e.g., as for cryopreservation compositions.

In some embodiments, the pharmaceutical composition comprises from or from about 1×10⁷ to or to about 1×10⁹ cells/mL. In some embodiments, the pharmaceutical composition comprises 1×10⁸ cells/mL. In some embodiments, the pharmaceutical composition comprises about 1×10⁸ cells/mL.

In some embodiments, the pharmaceutical composition further comprises an antibody or antigen binding fragment thereof, e.g., an antibody described herein.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

### V. METHODS OF TREATMENT

The NK cells described herein, e.g., the CAR-NK cells described herein, find use for treating cancer or other proliferative disorders.

Thus, also provided herein are methods of treating a patient suffering from a disorder, e.g., a disorder associated with a cancer, e.g., a CD19+ cancer, comprising administering the NK cells, e.g., the NK cells described herein, e.g., the CAR-NK cells described herein.

Also provided herein are methods of preventing, reducing and/or inhibiting the recurrence, growth, proliferation, migration and/or metastasis of a cancer cell or population of cancer cells in a subject in need thereof, comprising administering the NK cells, e.g., the NK cells described herein, e.g., the CAR-NK cells described herein.

Also provided herein are methods of enhancing, improving, and/or increasing the response to an anticancer therapy in a subject in need thereof, comprising administering the NK cells, e.g., the NK cells described herein, e.g., the CAR-NK cells described herein.

Also provided herein are methods for inducing the immune system in a subject in need thereof comprising administering the NK cells, e.g., the NK cells described herein, e.g., the CAR-NK cells described herein.

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. Generally, the methods include administering a therapeutically effective amount of a treatment as described herein, to a subject who is in need of, or who has been determined to be in need of, such treatment. In some embodiments, the methods include administering a therapeutically effective amount of a treatment comprising NK cells, e.g., CAR-NK cells described herein.

As used herein, the terms “treatment,” “treat,” and “treating” refer to reversing, alleviating, delaying the onset of, or inhibiting the progress of a disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, among other things. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “inhibition”, as it relates to cancer and/or cancer cell proliferation, refer to the inhibition of the growth, division, maturation or viability of cancer cells, and/or causing the death of cancer cells, individually or in aggregate with other cancer cells, by cytotoxicity, nutrient depletion, or the induction of apoptosis.

As used herein, “delaying” development of a disease or disorder, or one or more symptoms thereof, means to defer, hinder, slow, retard, stabilize and/or postpone development of the disease, disorder, or symptom thereof. This delay can be of varying lengths of time, depending on the history of the disease and/or subject being treated. As is evident to one skilled in the art, a sufficient or significant delay can, in effect, encompass prevention, in that the subject does not develop the disease, disorder, or symptom thereof. For example, a method that “delays” development of cancer is a method that reduces the probability of disease development in a given time frame and/or reduces extent of the disease in a given time frame, when compared to not using the method. Such comparisons may be based on clinical studies, using a statistically significant number of subjects.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject and/or before a certain stage of the disease (e.g., administration of a therapeutic substance to a subject with a cancer that has not yet metastasized). The subject may be an individual at risk of developing the disease or disorder, or at risk of disease progression, e.g., cancer metastasis. Such as an individual who has one or more risk factors known to be associated with development or onset of the disease or disorder. For example, an individual may be have mutations associated with the development or progression of a cancer. Further, it is understood that prevention may not result in complete protection against onset of the disease or disorder. In some instances, prevention includes reducing the risk of developing the disease or disorder. The reduction of the risk may not result in complete elimination of the risk of developing the disease or disorder.

An “increased” or “enhanced” amount (e.g., with respect to antitumor response, cancer cell metastasis) refers to an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein. It may also include an increase of at least 10%, at least 20%, at least 30%, at least 40%, at least 50%, at least 60%, at least 70%, at least 80%, at least 90%, at least 100%, at least 150%, at least 200%, at least 500%, or at least 1000% of an amount or level described herein.

A “decreased” or “reduced” or “lesser” amount (e.g., with respect to tumor size, cancer cell proliferation or growth) refers to a decrease that is about 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7, 1.8, etc.) an amount or level described herein. It may also include a decrease of at least 10%, at least 20%, at least 30%, at least 40%, at least 50%, at least 60%, at least 70%, at least 80%, or at least 90%, at least 100%, at least 150%, at least 200%, at least 500%, or at least 1000% of an amount or level described herein.

### A. Disorders

Methods and manufactured compositions disclosed herein find use in targeting a number of disorders, such as cellular proliferative disorders. A benefit of the approaches herein is that allogenic cells are used to target specific cells. Unlike previous therapies, such as chemo or radiotherapy, using the approaches and pharmaceutical compositions herein, one is able to specifically target cells exhibiting detrimental proliferative activity, potentially without administering a systemic drug or toxin that impacts proliferating cells indiscriminately.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease.

In some embodiments, the cancer is selected from the group consisting of: acute lymphoblastic leukemia (ALL), acute myeloid leukemia (AML), adrenocortical carcinoma, Kaposi sarcoma, AIDS-related lymphoma, primary CNS lymphoma, anal cancer, appendix cancer, astrocytoma, typical teratoid/rhabdoid tumor, basal cell carcinoma, bile duct cancer, bladder cancer, bone cancer, brain tumor, breast cancer, bronchial tumor, Burkitt lymphoma, carcinoid, cardiac tumors, medulloblastoma, germ cell tumor, primary CNS lymphoma, cervical cancer, cholangiocarcinoma, chordoma, chronic lymphocytic leukemia (CLL), chronic myelogenous leukemia (CML), chronic myeloproliferative neoplasms, colorectal cancer, craniopharyngioma, cutaneous T-cell lymphoma, ductal carcinoma in situ, embryonal tumors, endometrial cancer, ependymoma, esophageal cancer, esthesioneuroblastoma, Ewing sarcoma, extracranial germ cell tumor, extragonadal germ cell tumor, eye cancer (e.g., intraocular melanoma or retinoblastoma), fallopian tube cancer, fibrous histiocytoma of bone, osteosarcoma, gallbladder cancer, gastric cancer, gastrointestinal carcinoid tumor, gastrointestinal stromal tumors (GIST), germ cell tumors, gestational trophoblastic disease, hairy cell leukemia, head and neck cancer, heart tumor, hepatocellular cancer, histiocytosis, Hodgkin lymphomas, hypopharyngeal cancer, intraocular melanoma, islet cell tumors, pancreatic neuroendocrine tumors, kidney (renal cell) carcinoma, Langerhans cell histiocytosis, laryngeal cancer, leukemia, lip and oral cavity cancer, liver cancer, lung cancer (e.g., non-small cell lung cancer, small cell lung cancer, pleuropulmonary blastoma, and tracheobronchial tumor), lymphoma, male breast cancer, malignant fibrous histiocytoma of bone, melanoma, Merkel cell carcinoma, mesothelioma, metastatic cancer, metastatic squamous neck cancer, midline tract carcinoma, mouth cancer, multiple endocrine neoplasia syndromes, multiple myeloma/plasma cell neoplasms, mycosis fungoides, myelodysplastic syndromes, myelodysplastic/myeloproliferative neoplasms, myeloproliferative neoplasms, nasal cavity and paranasal sinus cancer, nasopharyngeal cancer, neuroblastoma, non-Hodgkin lymphoma, oral cancer, lip and oral cavity cancer, oropharyngeal cancer, osteosarcoma, malignant fibrous histiocytoma, ovarian cancer, pancreatic cancer, pancreatic neuroendocrine tumors, papillomatosis, paraganglioma, paranasal sinus and nasal cavity cancer, parathyroid cancer, penile cancer, pharyngeal cancer, pheochromocytomas, pituitary tumor, plasma cell neoplasm, multiple myeloma, pleuropulmonary blastoma, pregnancy and breast cancer, primary central nervous system lymphoma, primary peritoneal cancer, prostate cancer, rectal cancer, recurrent cancer, renal cell cancer, retinoblastoma, rhabdomyosarcoma, salivary gland cancer, sarcoma (e.g., childhood rhabdomyosarcoma, childhood vascular tumors, Ewing sarcoma, Kaposi sarcoma, osteosarcoma, soft tissue sarcoma, uterine sarcoma), Sezary syndrome, skin cancer, small intestine cancer, soft tissue sarcoma, squamous cell carcinoma, squamous neck cancer, stomach cancer, T-cell lymphomas, testicular cancer, throat cancer, nasopharyngeal cancer, oropharyngeal cancer, hypopharyngeal cancer, thryomoma and thymic carcinomas, thyroid cancer, tracheobronchial tumors, transitional cell cancer of the renal pelvis and ureter, urethral cancer, uterine cancer, uterine sarcoma, vaginal cancer, vascular tumors, vulvar cancer, and Wilms tumor.

In some embodiments, the cancer is a solid tumor. In some embodiments, the cancer is metastatic.

The methods described herein include methods for the treatment of disorders associated with a cancer, e.g., a CD19+ cancer, e.g., a B-cell malignancy. In some embodiments, the disorder is selected from the group consisting of acute lymphoblastic leukemia (ALL), B-cell lymphoma, B-cell leukemia, and combinations thereof. In some embodiments, the disorder is a heme malignancy. In some embodiments, the heme malignancy is an advanced heme malignancy.

In some embodiments, the disorder is selected from the group consisting of Non-Hodgkin lymphoma (NHL), diffuse large B-cell lymphoma (DLBCL), follicular lymphoma, chronic lymphocytic leukemia (CLL), small lymphocytic lymphoma (SLL), mantle cell lymphoma (MCL), marginal zone lymphoma, Burkitt lymphoma, Burkitt-like lymphoma, lymphoplasmacytic lymphoma (Waldenstrom macroglobulinemia), hairy cell leukemia, primary central nervous system (CNS) lymphoma, primary intraocular lymphoma (lymphoma of the eye), and combinations thereof.

In some embodiments, the marginal zone lymphoma is selected from the group consisting of extranodal marginal zone B-cell lymphoma (also known as mucosa-associated lymphoid tissue (MALT) lymphoma, nodal marginal zone B-cell lymphoma, splenic marginal zone B-cell lymphoma, and combinations thereof.

In some embodiments, the cancer is a CD20+ cancer. In some embodiments, the CD20+ cancer is selected from the group consisting of indolent or aggressive non-Hodgkin's lymphoma (NHL). In some embodiments, the CD20+ cancer is relapsed or refractory indolent or aggressive NHL of B-cell origin. Among the aggressive and indolent subtypes are those in Table 7. In some embodiments, the cancer is both CD19+ and CD20+.

TABLE 7
Exemplary Aggressive and Indolent

| Aggressive Subtype | Indolent Subtype |
|---|---|
| Diffuse large B-cell lymphoma | Follicular lymphoma (Grades I, II, and IIIA) |
| Mantle cell lymphoma | Lymphoplasmacytic lymphoma/Waldenström |
| | macroglobulinemia |
| Transformed follicular lymphoma | Gastric MALT (MZL) |
| Follicular lymphoma (Grade IIIB) | Non-gastric MALT (MZL) |
| Transformed mucosa-associated lymphoid | Nodal marginal zone lymphoma (MZL) |
| tissue (MALT) lymphoma | |
| Primary mediastinal B-cell lymphoma | Splenic marginal zone lymphoma (MZL) |
| Lymphoblastic lymphoma | Small-cell lymphocytic lymphoma (SLL)/Chronic |
| | lymphocytic lymphoma (CLL) with nodal or |
| | splenic involvement |
| High-grade B-cell lymphomas with | |
| translocations of MYC and BCL2 and/or | |
| BCL6 (double/triple hit lymphoma) | |

### B. Patients

Suitable patients for the compositions and methods herein include those who are suffering from, who have been diagnosed with, or who are suspected of having a cellular proliferative and/or differentiative disorder, e.g., a cancer. Patients subjected to technology of the disclosure herein generally respond better to the methods and compositions herein, in part because the pharmaceutical compositions are allogeneic and target cells identified by the antigen binding domain, rather than targeting proliferating cells generally. As a result, there is less off-target impact and the patients are more likely to complete treatment regimens without substantial detrimental off-target effects.

In some embodiments, the methods of treatment provided herein may be used to treat a subject (e.g., human, monkey, dog, cat, mouse) who has been diagnosed with or is suspected of having a cellular proliferative and/or differentiative disorder, e.g., a cancer. In some embodiments, the subject is a mammal. In some embodiments, the subject is a human.

As used herein, a subject refers to a mammal, including, for example, a human.

In some embodiments, the mammal is selected from the group consisting of an armadillo, an ass, a bat, a bear, a beaver, a cat, a chimpanzee, a cow, a coyote, a deer, a dog, a dolphin, an elephant, a fox, a panda, a gibbon, a giraffe, a goat, a gopher, a hedgehog, a hippopotamus, a horse, a humpback whale, a jaguar, a kangaroo, a koala, a leopard, a lion, a llama, a lynx, a mole, a monkey, a mouse, a narwhal, an orangutan, an orca, an otter, an ox, a pig, a polar bear, a porcupine, a puma, a rabbit, a raccoon, a rat, a rhinoceros, a sheep, a squirrel, a tiger, a walrus, a weasel, a wolf, a zebra, a goat, a horse, and combinations thereof.

In some embodiments, the mammal is a human.

The subject, e.g., the human subject, can be a child, e.g., from or from about 0 to or to about 14 years in age. The subject can be a youth, e.g., from or from about 15 to or to about 24 years in age. The subject can be an adult, e.g., from or from about 25 to or to about 64 years in age. The subject can be a senior, e.g, 65+ years in age.

In some embodiments, the subject may be a human who exhibits one or more symptoms associated with a cellular proliferative and/or differentiative disorder, e.g., a cancer, e.g., a tumor. Any of the methods of treatment provided herein may be used to treat cancer at various stages. By way of example, the cancer stage includes but is not limited to early, advanced, locally advanced, remission, refractory, reoccurred after remission and progressive. In some embodiments, the subject is at an early stage of a cancer. In other embodiments, the subject is at an advanced stage of cancer. In various embodiments, the subject has a stage I, stage II, stage III or stage IV cancer. The methods of treatment described herein can promote reduction or retraction of a tumor, decrease or inhibit tumor growth or cancer cell proliferation, and/or induce, increase or promote tumor cell killing. In some embodiments, the subject is in cancer remission. The methods of treatment described herein can prevent or delay metastasis or recurrence of cancer.

In some embodiments, the subject is at risk, or genetically or otherwise predisposed (e.g., risk factor), to developing a cellular proliferative and/or differentiative disorder, e.g., a cancer, that has or has not been diagnosed.

As used herein, an “at risk” individual is an individual who is at risk of developing a condition to be treated, e.g., a cellular proliferative and/or differentiative disorder, e.g., a cancer. Generally, an “at risk” subject may or may not have detectable disease, and may or may not have displayed detectable disease prior to the treatment methods described herein. “At risk” denotes that an individual has one or more so-called risk factors, which are measurable parameters that correlate with development of a disease or condition and are known in the art. For example, an at risk subject may have one or more risk factors, which are measurable parameters that correlate with development of cancer. A subject having one or more of these risk factors has a higher probability of developing cancer than an individual without these risk factor(s). In general, risk factors may include, for example, age, sex, race, diet, history of previous disease, presence of precursor disease, genetic (e.g., hereditary) considerations, and environmental exposure. In some embodiments, the subjects at risk for cancer include, for example, those having relatives who have experienced the disease, and those whose risk is determined by analysis of genetic or biochemical markers.

In addition, the subject may be undergoing one or more standard therapies, such as chemotherapy, radiotherapy, immunotherapy, surgery, or combination thereof. Accordingly, one or more kinase inhibitors may be administered before, during, or after administration of chemotherapy, radiotherapy, immunotherapy, surgery or combination thereof.

In certain embodiments, the subject may be a human who is (i) substantially refractory to at least one chemotherapy treatment, or (ii) is in relapse after treatment with chemotherapy, or both (i) and (ii). In some of embodiments, the subject is refractory to at least two, at least three, or at least four chemotherapy treatments (including standard or experimental chemotherapies).

### C. Lymphodepletion

In some embodiments, the patient is lymphodepleted before treatment. Illustrative lymphodepleting chemotherapy regimens, along with correlative beneficial biomarkers, are described in WO 2016/191756 and WO 2019/079564, hereby incorporated by reference in their entirety. In certain embodiments, the lymphodepleting chemotherapy regimen comprises administering to the patient doses of cyclophosphamide (between 200 mg/m²/day and 2000 mg/m²/day) and doses of fludarabine (between 20 mg/m²/day and 900 mg/m²/day).

In some embodiments, lymphodepletion comprises administration of or of about 250 to about 500 mg/m² of cyclophosphamide, e.g., from or from about 250 to or to about 500, 250, 400, 500, about 250, about 400, or about 500 mg/m² of cyclophosphamide. In some embodiments, lymphodepletion comprises administration of or of about 20 mg/m²/day to or to about 40 mg/m²/day fludarabine, e.g., 30 or about 30 mg/m²/day. In some embodiments, lymphodepletion comprises administration of both cyclophosmamide and fludarabine. In some embodiments, the patient is lymphodepleted by intravenous administration of cyclophosphamide (250 mg/m²/day) and fludarabine (30 mg/m²/day). In some embodiments, the patient is lymphodepleted by intravenous administration of cyclophosphamide (500 mg/m²/day) and fludarabine (30 mg/m²/day). In some embodiments, the lymphodepletion occurs no more than 5 days prior to the first dose of NK cells. In some embodiments, the lymphodepletion occurs no more than 7 days prior to the first dose of NK cells. In some embodiments, lymphodepletion occurs daily for 3 consecutive days, starting 5 days before the first dose of NK cells (i.e., from Day −5 through Day −3). In some embodiments, the lymphodepletion occurs on day −5, day −4 and day −3.

### D. Administration

### 1. NK Cells

In some embodiments, the NK cells, e.g., the NK cells described herein, e.g., the CAR-NK cells described herein are administered as part of a pharmaceutical composition, e.g., a pharmaceutical composition described herein. Cells are administered after thawing, in some cases without any further manipulation in cases where their cryoprotectant is compatible for immediate administration. For a given individual, a treatment regimen often comprises administration over time of multiple aliquots or doses of NK cells, which can be drawn from a common batch or donor.

In some embodiments, the NK cells, e.g., the NK cells described herein, e.g., the CAR-NK cells described herein, are administered at or at about 5×10⁶ to or to about 1×10⁹ NK cells per dose. In some embodiments, the NK cells are administered at or at about 1×10⁶, at or at about 1×10⁷, at or at about 3×10⁷, at or at about 1×10⁸, at or at about 3×10⁸, or at or at about 1×10⁹ cells per dose.

The ability to offer repeat dosing may allow patients to experience or maintain a deeper or prolonged response from the therapy. For example, patients can receive response-based dosing, during which the patient continues to receive doses of CAR-NK cell therapy for as long as the patient derives a benefit. The number of doses and the number of cells administered in each dose can also be tailored to the individual patient. Thus, the CAR-NK cell therapies described herein can be tailored to each patient based on that patient's own response. Examples of patient responses include partial responses and complete responses. In some cases, the therapy can be terminated if the patient no longer derives a benefit from the CAR-NK cell therapy. In some cases, the therapy can also be reinitiated if the patient relapses. In some embodiments, therapy can last a year.

In some embodiments, the NK cells are administered weekly. In some embodiments, the NK cells are administered monthly. In some embodiments, the NK cells are administered every other month or once every three months. In some embodiments, the NK cells are administered for or for about 8 weeks. In some embodiments, the NK cells are administered between one and four times over the course of nine months.

In some embodiments, the NK cells are cryopreserved in an infusion-ready media, e.g., a cryopreservation composition suitable for intravenous administration, e.g., as described herein. In some embodiments, the NK cells are cryopreserved in vials containing from or from about 1×10⁷ to or to about 1×10⁹ cells per vial. In some embodiments, the NK cells are cryopreserved in vials containing a single dose. In some embodiments, the cells are thawed, e.g., in a 37° C. water bath, prior to administration. In some embodiments, the thawed vial(s) of NK cells are aseptically transferred to a single administration vessel, e.g., administration bag using, e.g., a vial adapter and a sterile syringe. The NK cells can be administered to the patient from the vessel through a Y-type blood/solution set filter as an IV infusion, by gravity. In some embodiments, the NK cells are administered as soon as practical, preferably less than 90 minutes, e.g., less than 80, 70, 60, 50, 40, 30, 20, or 10 minutes after thawing. In some embodiments, the NK cells are administered within 30 minutes of thawing. In some embodiments, the pharmaceutical composition is administered intravenously via syringe. In some embodiments, 1 mL, 4 mL, or 10 mL of drug product is administered to the patient intravenously via syringe.

### 2. Cytokines

In some embodiments, a cytokine is administered to the patient.

In some embodiments, the cytokine is administered together with the NK cells as part of a pharmaceutical composition. In some embodiments, the cytokine is administered separately from the NK cells, e.g., as part of a separate pharmaceutical composition.

In some embodiments, the cytokine is IL-2. In some embodiments, the IL-2 is administered subcutaneously. In some embodiments, the IL-2 is administered from between 1 to 4 or about 1 to about 4 hours following the conclusion of NK cell administration. In some embodiments, the IL-2 is administered at least 1 hour following the conclusion of NK cell administration. In some embodiments, the IL-2 is administered no more than 4 hours following the conclusion of NK cell administration. In some embodiments, the IL-2 is administered at least 1 hour after and no more than 4 hours following the conclusion of NK cell administration.

In some embodiments, the IL-2 is administered at up to 10 million IU/M², e.g., up to 1 million, 2 million, 3 million, 4 million, 5 million, 6 million, 7 million, 8 million, 9 million, or 10 million IU/m². In some embodiments, the IL-2 is administered at or at about 1 million, at or at about 2 million, at or at about 3 million, at or at about 4 million, at or at about 5 million, at or at about 6 million, at or at about 7 million, at or at about 8 million, at or at about 9 million, at or at about 10 million IU/M². In some embodiments, the IL-2 is administered at or at about 1×10⁶ IU/M². In some embodiments, the IL-2 is administered at or at about 2×10⁶ IU/M². In some embodiments, less than 1×10⁶ IU/M²IL-2 is administered to the patient. In some embodiments, a flat dose of IL-2 is administered to the patient. In some embodiments, a flat dose of 6 million IU or about 6 million IU is administered to the patient.

In some embodiments, IL-2 is not administered to the patient.

### E. Dosing

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### F. Combination Therapies

In some embodiments, the method comprises administering the NK cells described herein, e.g., the CAR-NK cells described herein, in combination with another therapy, e.g., an antibody, an NK cell engager, an antibody drug conjugate (ADC), a chemotherapy drug, e.g., a small molecule drug, an immune checkpoint inhibitor, and combinations thereof.

### 1. Antibodies

In some embodiments, the other therapy is an antibody.

In some embodiments, the antibody binds to a target selected from the group consisting of CD20, HER-2, EGFR, CD38, SLAMF7, GD2, ALKI, AMHR2, CCR2, CD137, CD19, CD26, CD32b, CD33, CD37, CD70, CD73, CD74, CD248, CLDN6, Clever-1, c-MET, CSF-1R, CXCR4, DKK1, DR5, Epha3, FGFR2b, FGFR3, FLT3, FOLR1, Globo-H, Glypican3, GM1, Grp78, HER-3, HGF, IGF-1R, ILIRAP, IL-8R, ILT4, Integrin alpha V, M-CSF, Mesothelin, MIF, MUC1, MUC16, MUC5AC, Myostatin, NKG2A, NOTCH, NOTCH2/3, PIGF, PRL3, PSMA, ROR1, SEMA4D, Sialyl Lewis A, Siglec15, TGF-b, TNFR3, TRAIL-R2, VEGF, VEGFR1, VEGFR2, Vimentin, and combinations thereof.

Suitable antibodies include, but are not limited to those shown in Table 8.

TABLE 8
Antibodies for Combination Therapy

| Target | Drug Name | Brand Name | Indication(s) | Reference |
|---|---|---|---|---|
| CD20 | Rituxan | Rituximab | DLBCL/FL, NHL, | Du et al., Auto Immun Highlights |
| | | | CLL, RA, GPA, | (2017) 8(1): 12 |
| | | | MPA | |
| CD20 | Gazyva | Obinutuzumab | CLL, FL | Gagez et al., Curr Opin Oncol. |
| | | | | 2014 September; 26(5): 484-91 |
| CD20 | Arzerra | Ofatumumab | CLL | Robak, Curr Opin Mol Ther. |
| | | | | 2008 June; 10(3): 294-309 |
| CD20 | Ocrevus | Ocrelizumab | RMS, PPMS | Genovese et al., Arthritis Rheum. |
| | | | | 2008 September; 58(9): 2652-61 |
| CD20 | Zevalin | Ibritumomab | NHL | Wiseman et al., Eur J Nucl Med. |
| | | | | 2000 July; 27(7): 766-77 |
| CD20 | | Veltuzumab | NHL, CLL | Kalaycio et al. Leuk Lymphoma. |
| | | | | 2016; 57(4): 803-11 |
| CD20 | Bexxar | Tositumomab | NHL | Vose et al., J Clin Oncol. |
| | | and Iodine I 131 | | 2000 March; 18(6): 1316-23 |
| | | tositumomab | | |
| CD20 | | Ublituximab | NHL, CLL, RMS | Sawas et al., Br J Haematol. |
| | | | | 2017 April; 177(2): 243-253 |
| NKG2A | | Monalizumab | | Hall et al., J Immunother Cancer |
| | | | | 2019; (7): 263 |

In some embodiments, the CD20 targeting antibody is a CD20 targeting antibody selected from Table 9, or a combination thereof.

TABLE 9
CD20 Targeted Antibodies

| Antibody | Internal Name | Antigen | Company | Reference |
|---|---|---|---|---|
| ofatumumab | Arzerra, Kesimpta, | CD20 | Genmab, | Sorensen et al., Neurology. |
| | GSK1841157, | | GSK, | 2014 Feb. 18; 82(7): 573-81 |
| | HuMax-CD20, 2F2, | | Novartis | |
| | OMB157 | | | |
| ibritumomab | Zevalin, 2B8, C2B8, | CD20 | Biogen, | Witzig et al., J Clin Oncol. |
| tiuxetan | Y2B8 | | CTI | 2002 May 15; 20(10): 2453-63 |
| | | | Biopharma, | |
| | | | Spectrum | |
| rituximab | MabThera, Rituxan, | CD20 | Biogen, | Salles et al., Adv Ther. |
| | C2B8, IDEC-C2B8, | | Roche | 2017 October; 34(10): 2232-2273 |
| | IDEC-102, RG105 | | | |
| obinutuzumab | Gazyvaro, Gazyva, | CD20 | Biogen, | Marcus et al., N Engl J Med. |
| | GA101, GA-101, | | Genentech, | 2017 Oct. 5; 377(14): 1331-1344 |
| | RO5072759, RG7159, | | Glycart, | |
| | R7159, humanized | | Roche | |
| | B-Ly1, afatuzumab | | | |
| tositumomab | Bexxar, I131 | CD20 | GSK | Zelenetz, Semin Oncol. |
| | tositumomab | | | 2003 April; 30(2 Suppl 4): 22-30 |
| ocrelizumab | Ocrevus, 2H7.v16, | CD20 | Biogen, | Kappos et al., Lancet. |
| | rhuMAb2H7, | | Genentech, | 2011 Nov. 19; 378(9805): 1779-87 |
| | PRO70769, RG1594 | | Xoma | |
| GP2013 | Riximyo, SDZ-RTX, | CD20 | Novartis, | Smolen et al., Ann Rheum Dis. |
| | Rixathon | | Sandoz | 2017 September; 76(9): 1598-1602 |
| rituximab- | Truxima, Tuxella, | CD20 | Celltrion, | Coiffier, Expert Rev Clin Pharmacol. |
| abbs | Ritemvia, Blitzima, | | Mundipharma | 2017 September; 10(9): 923-933 |
| | CT-P10 | | | |
| BCD-020 | AcellBia, BCD020 | CD20 | Biocad | Poddubnaya et al., Hematol Oncol. |
| | | | | 2020 February; 38(1): 67-73 |
| HLX01 | Hanlikon | CD20 | Shanghai | Shi et al., J Hematol Oncol. |
| | | | Henlius | 2020 Apr. 16; 13(1): 38 |
| IGN002 | | CD20 | ImmunGene, | Trial ID: NCT02847949 |
| | | | Valor Bio | |
| MK-8808 | | CD20 | Merck (MSD) | Trial ID: NCT01370694 |
| plamotamab | XmAb13676, | CD20, | Novartis, | Trial ID: NCT02924402 |
| | XENP13676 | CD3 | Xencor | |
| MT-3724 | | CD20 | Molecular | Huang et al., Blood Cancer J. |
| | | | Templates | 2018 Mar. 20; 8(3): 33 |
| RGB-03 | | CD20 | Gedeon Richter | Trial ID: NCT02371096 |
| IGM-2323 | | CD20, | IGM Bio | Trial ID: NCT04082936 |
| | | CD3 | | |
| CHO-H01 | | CD20 | Cho Pharma | Trial ID: NCT03221348 |
| B001 | | CD20 | Shanghai | Trial ID: NCT03332121 |
| | | | Pharma | |
| | | | Holdings | |
| BCD-132 | | CD20 | Biocad | Trial ID: NCT04056897 |
| Sunshine | | CD20 | Sunshine | Trial ID: NCT03980379 |
| Guojian 304 | | | Guojian | |
| | | | Pharma | |
| IMM0306 | | CD20, | ImmuneOnco | Trial ID: NCT04746131 |
| | | CD47 | | |
| ocaratuzumab | AME 33, AME-133v, | CD20 | AME, Lilly, | Cheney et al., MAbs. |
| | LY2469298 | | Mentrik | May-June 2014; 6(3): 749-55 |
| PRO131921 | | CD20 | Genentech | Casulo et al., Clin Immunol. |
| | | | | 2014 September; 154(1): 37-46 |
| TL011 | | CD20 | Teva | Trial ID: NCT01205737 |
| mosunetuzumab | BTCT4465A, | CD20, | Genentech | Hosseini et al., NPJ Syst Biol Appl. |
| | RG7828, RO7030816 | CD3e | | 2020 Aug. 28; 6(1): 28 |
| 2B8T2M | ALT-803 | CD20, | Altor | Trial ID: NCT01946789 |
| | | IL-15 | | |
| MIL62 | | CD20 | Beijing | Trial ID: NCT04103905 |
| | | | Mabworks | |
| TQB2303 | | CD20 | Chia Tai | Trial ID: NCT03777085 |
| | | | Tianqing | |
| | | | Pharma | |
| SBI-087 | PF-05230895, | CD20 | Pfizer, | Damjanov et al., J Rheumatol. |
| | 2LM20-4 | | Trubion | 2016 December; 43(12): 2094-2100 |
| TRU-015 | PF-5212374 | CD20 | Pfizer, | Burge et al., Clin Ther. |
| | | | Trubion | 2008 October; 30(10): 1806-16 |
| veltuzumab | hA20, IMMU-106 | CD20 | Immunomedics, | Goldenberg et al., Leuk Lymphoma. |
| | | | Nycomed, | 2010 May; 51(5): 747-55 |
| | | | Takeda | |
| odronextamab | REGN1979 | CD20, | Regeneron | Trial ID: NCT03888105 |
| | | CD3 | | |
| RO7082859 | CD20-TCB | CD20 | Roche | Trial ID: NCT03075696 |
| zuberitamab | HS006, | CD20 | Zheijang Hisun | Trial ID: NCT03485118 |
| | RHCACD20MA | | | |
| PBO-326 | | CD20 | Probiomed | Trial ID: NCT01277172 |
| ublituximab | LFB-R603, TGTX- | CD20 | LFB, TG | Fox et al., Mult Scler. |
| | 1101, TG-1101 | | Therapeutics | 2021 March; 27(3): 420-429 |
| Reditux | DRL_RI | CD20 | Dr. Reddy's | Bhati et al., Clin Rheumatol. |
| | | | | 2016 August; 35(8): 1931-1935 |
| CMAB304 | Retuxira | CD20 | Shanghai CP | Trial ID: NCT01459887 |
| | | | Guojian | |
| PF-05280586 | Rituximab-Pfizer | CD20 | Pfizer | Sharman et al., BioDrugs. |
| | | | | 2020 April; 34(2): 171-181 |
| BI 695500 | | CD20 | Boehringer | Trial ID: NCT01950273 |
| ripertamab | SCT400 | CD20 | Sinocelltech | Trial ID: NCT02206308 |
| ABP 798 | ABP798 | CD20 | Amgen | Niederwieser et al., Target Oncol. |
| | | | | 2020 October; 15(5): 599-611 |
| IBI301 | IBI301-A | CD20 | Innovent, | Jiang et al., Sci Rep. |
| | | | Lilly | 2020 Jul. 15; 10(1): 11676 |
| MabionCD20 | | CD20 | Mabion | Trial ID: NCT02617485 |
| RTXM83 | | CD20 | mAbxience | Cerutti et al., BioDrugs. |
| | | | | 2019 June; 33(3): 307-319 |
| SAIT101 | | CD20 | Samsung | Trial ID: NCT02809053 |
| | | | Bioepis | |
| epcoritamab | GEN3013, DuoBody- | CD20, | Abbvie, | Van der Horst et al., Blood Cancer J. |
| | CD3xCD20 | CD3 | Genmab | 2021 Feb. 18; 11(2): 38 |
| GB241 | | CD20 | Genor | Trial ID: NCT03003039 |
| JHL1101 | | CD20 | JHL Biotech | Trial ID: NCT03670901 |

In some embodiments, the CD20 targeting antibody is selected from the group comprising rituximab (or a biosimilar thereof), obinutuzumab (or a biosimilar thereof), ofatumumab (or a biosimilar thereof), ocrelizumab (or a biosimilar thereof), ibritumomab (or a biosimilar thereof), veltuzumab (or a biosimilar thereof), tositumomab (or a biosimilar thereof), ublituximab (or a biosimilar thereof), and combinations thereof.

### 2. Small Molecule/Chemotherapy Drugs

In some embodiments, the additional therapy is a small molecule drug. In some embodiments, the additional therapy is a chemotherapy drug. In some embodiments, the additional therapy is a small molecule chemotherapy drug. Such small molecule drugs can include existing standard-of-care treatment regimens to which adoptive NK cell therapy is added. In some cases, the use of the NK cells described herein can enhance the effects of small molecule drugs, including by enhancing the efficacy, reducing the amount of small molecule drug necessary to achieve a desired effect, or reducing the toxicity of the small molecule drug.

In some embodiments, the drug is selected from the group consisting of

In some embodiments, the drug is [(1S,2S,3R,4S,7R,9S,10S,12R,15S)-4-acetyloxy-1,9,12-trihydroxy-15-[(2R,3S)-2-hydroxy-3-[(2-methylpropan-2-yl)oxycarbonylanino]-3-phenylpropanoyl]oxy-10,14,17,17-tetramethyl-11-oxo-6-oxatetracyclo[11.3.1.0^{3,10}.0^{4,7}]heptadec-13-en-2-yl] benzoate (docetaxel) or a pharmaceutically acceptable salt thereof.

In some embodiments, the drug is [(1S,2S,3R,4S,7R,9S,10S,12R,15S)-4,12-diacetyloxy-15-[(2R,3S)-3-benzamido-2-hydroxy-3-phenylpropanoyl]oxy-1,9-dihydroxy-10,14,17,17-tetramethyl-11-oxo-6-oxatetracyclo[11.3.1.0^{3,10}.0^{4,7}]heptadec-13-en-2-yl] benzoate (paclitaxel) or a pharmaceutically acceptable salt thereof.

In some embodiments, the drug is 6-N-(4,4-dimethyl-5H-1,3-oxazol-2-yl)-4-N-[3-methyl-4-([1,2,4]triazolo[1,5-a]pyridin-7-yloxy)phenyl]quinazoline-4,6-diamine (tucatinib) or a pharmaceutically acceptable salt thereof.

In some embodiments, the drug is pentyl N-[1-[(2R,3R,4S,5R)-3,4-dihydroxy-5-methyloxolan-2-yl]-5-fluoro-2-oxopyrimidin-4-yl]carbamate (capecitabine) or a pharmaceutically acceptable salt thereof.

In some embodiments, the drug is azanide;cyclobutane-1,1-dicarboxylic acid;platinum(2+) (carboplatin) or a pharmaceutically acceptable salt thereof.

In some embodiments, the drug is methyl (1R,9R,10S,11R,12R,19R)-11-acetyloxy-12-ethyl-4-[(12S,14R)-16-ethyl-12-methoxycarbonyl-1,10-diazatetracyclo[12.3.1.0^{3,11}.0^{4,9}]octadeca-3(11),4,6,8,15-pentaen-12-yl]-10-hydroxy-5-methoxy-8-methyl-8,16-diazapentacyclo[10.6.1.0^{1,9}.0^{2,7}.0^{16,19}]nonadeca-2,4,6,13-tetraene-10-carboxylate (vinorelbine) or a pharmaceutically acceptable salt thereof.

In some embodiments, the drug is N-[3-chloro-4-[(3-fluorophenyl)methoxy]phenyl]-6-[5-[(2-methylsulfonylethylamino)methyl]furan-2-yl]quinazolin-4-amine (lapatinib) or a pharmaceutically acceptable salt thereof.

In some embodiments, the drug is (E)-N-[4-[3-chloro-4-(pyridin-2-ylmethoxy)anilino]-3-cyano-7-ethoxyquinolin-6-yl]-4-(dimethylamino)but-2-enamide (neratinib) or a pharmaceutically acceptable salt thereof.

In some embodiments, the drug is 6-acetyl-8-cyclopentyl-5-methyl-2-[(5-piperazin-1-ylpyridin-2-yl)amino]pyrido[2,3-d]pyrimidin-7-one (palbociclib) or a pharmaceutically acceptable salt thereof.

In some embodiments, the drug is 7-cyclopentyl-N,N-dimethyl-2-[(5-piperazin-1-ylpyridin-2-yl)amino]pyrrolo[2,3-d]pyrimidine-6-carboxamide (ribociclib) or a pharmaceutically acceptable salt thereof.

In some embodiments, the drug is N-[5-[(4-ethylpiperazin-1-yl)methyl]pyridin-2-1]-5-fluoro-4-(7-fluoro-2-methyl-3-propan-2-ylbenzimidazol-5-yl)pyrimidin-2-amine (abemaciclib) or a pharmaceutically acceptable salt thereof.

In some embodiments, the drug is (1R,9S,12S,15R,16E,18R,19R,21R,23S,24E,26E,28E,30S,32S,35R)-1,18-dihydroxy-12-[(2R)-1-[(1S,3R,4R)-4-(2-hydroxyethoxy)-3-methoxycycclohexyl]propan-2-yl]-19,30-dimethoxy-15,17,21,23,29,35-hexamethyl-11,36-dioxa-4-azatricyclo[30.3.1.0^{4,9}]hexatriaconta-16,24,26,28-tetraene-2,3,10,14,20-pentone (everolimus) or a pharmaceutically acceptable salt thereof.

In some embodiments, the drug is (2S)-1-N-[4-methyl-5-[2-(1,1,1-trifluoro-2-methylpropan-2-yl)pyridin-4-yl]-1,3-thiazol-2-yl]pyrrolidine-1,2-dicarboxanide (alpelisib) or a pharmaceutically acceptable salt thereof.

In some embodiments, the drug is 4-[[3-[4-(cyclopropanecarbonyl)piperazine-1-carbonyl]-4-fluorophenyl]methyl]-2H-phthalazin-1-one (olaparib) or a pharmaceutically acceptable salt thereof.

In some embodiments, the drug is (11S,12R)-7-fluoro-11-(4-fluorophenyl)-12-(2-methyl-1,2,4-triazol-3-yl)-2,3,10-triazatricyclo[7.3.1.0.^{5,13}]trideca-1,5(13),6,8-tetraen-4-one (talazoparib) or a pharmaceutically acceptable salt thereof.

In some embodiments, the drug is N-[2-[2-(dimethylamino)ethyl-methylamino]-methoxy-5-[[4-(1-methylindol-3-yl)pyrimidin-2-yl]amino]phenyl]prop-2-enamid (osimertinib) or a pharmaceutically acceptable salt thereof.

In some embodiments, the drug is N-(3-chloro-4-fluorophenyl)-7-methoxy-6-(3-morpholin-4-ylpropoxy)quinazolin-4-amine (gefitinib) or a pharmaceutically acceptable salt thereof.

In some embodiments, the drug is N-3-ethynylphenyl)-6,7-bis(2-methoxyethoxy)quinazolin-4-amine (erlotinib) or a pharmaceutically acceptable salt thereof.

In some embodiments, the drug is (E)-N4-(3-chloro-4-fluoroanilino)-7-[(3S)-oxolan-3-yl]oxyquinazolin-6-yl]-4-(dimethylamino)but-2-enamide (afatinib) or a pharmaceutically acceptable salt thereof.

In some embodiments, the drug is azane;dichloroplatinum (cisplatin, platinol) or a pharmaceutically acceptable salt thereof.

In some embodiments, the drug is azanide;cyclobutane-1,1-dicarboxylic acid;platinum(2+) (carboplatin) or a pharmaceutically acceptable salt thereof.

In some embodiments, the drug is 4-amino-1-[(2R,4R,5R)-3,3-difluoro-4-hydroxy-5-(hydroxymethyl)oxolan-2-yl]pyrimidin-2-one (gemcitabine) or a pharmaceutically acceptable salt thereof.

In some embodiments, the drug is (25)-2-[[4-[2-(2-amino-4-oxo-3,7-dihydropyrrolo[2,3-d]pyrimidin-5-yl)ethyl]benzoyl]amino]pentanedioic acid (pemetrexed) or a pharmaceutically acceptable salt thereof.

In some embodiments, the drug is N,N-bis(2-chloroethyl)-2-oxo-1,3,2λ⁵-oxazaphosphinan-2-amine (cyclophosphamide) or a pharmaceutically acceptable salt thereof.

In some embodiments, the drug is (2R,3,S,5R)-2-(6-amino-2-fluoropurin-9-yl)-5-(hydroxymethyl)oxolane-3,4-diol (fludarabine) or a pharmaceutically acceptable salt thereof.

In some embodiments, the drug is (7S,9S)-7-[(2R,4S,5S,6S)-4-amino-5-hydroxy-6-methyloxan-2-yl]oxy-6,9,11-trihydroxy-9-(2-hydroxyacetyl)-4-methoxy-8,10-dihydro-7H-tetracene-5,12-dione (doxorubicin) or a pharmaceutically acceptable salt thereof.

In some embodiments, the drug is methyl (1R,9R,10S,11R,12R,19R)-11-acetyloxy-12-ethyl-4-[(13S,15S,17S)-17-ethyl-17-hydroxy-13-methoxycarbonyl-11-diazatetracyclo[13.3.1.0^{4,12}.0^{5,10}]nonadeca-4(12),5,7,9-tetraen-13-yl]-8-formyl-10-hydroxy-5-methoxy-8,16-diazapentacyclo[10.6.1.0^{1,9}.0^{2,7}.0^{16,19}]nonadeca-2,4,6,13-tetraene-10-carboxylate (vincristine) or a pharmaceutically acceptable salt thereof.

In some embodiments, the drug is (8S,9S,10R,13S,14S,17R)-17-hydroxy-17-(2-hydroxyacetyl)-10,13-dimethyl-6,7,8,9,12,14,15,16-octahydrocyclopenta[a]phenanthrene-3,11-dione (prednisone) or a pharmaceutically acceptable salt thereof.

In some embodiments, the drug is N,3-bis(2-chloroethyl)-2-oxo-1,3,2λ⁵ oxazaphosphinan-2-amine (ifosfamide) or a pharmaceutically acceptable salt thereof.

In some embodiments, the drug is (5S,5aR,8aR,9R)-5-[[(2R,4aR,6R,7R,8R,8aS)-7,8-dihydroxy-2-methyl-4,4a,6,7,8,8a-hexahydropyrano[3,2-d[1,3]dioxin-6-yl]oxy]-9-(4-hydroxy-3,5-dimethoxyphenyl)-5a,6,8a,9-tetrahydro-5H-[2]benzofuro[6,5-f][1,3]benzodioxol-8-one (etopside) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (dexamethasone) or a pharmaceutically acceptable salt thereof.

In some embodiments, the drug is (8S,9R,10S,11S,13S,14S,16R,17R)-9-fluoro-11,17-dihydroxy-17-(2-hydroxyacetyl)-10,13,16-trimethyl-6,7,8,11,12,14,15,16-octahydrocyclopenta[a]phenanthren-3-one (cytarabine) or a pharmaceutically acceptable salt thereof.

### 3. NK Cell Engagers

In some embodiments, the additional therapy is an NK cell engager, e.g., a bispecific or trispecific antibody.

In some embodiments, the NK cell engager is a bispecific antibody against CD16 and a disease-associated antigen, e.g., cancer-associated antigen, e.g., an antigen of cancers described herein, e.g., CD19. In some embodiments, the NK cell engager is a trispecific antibody against CD16 and two disease-associated antigens, e.g., cancer-associated antigens, e.g., antigens of cancers described herein.

### 4. Checkpoint Inhibitors

In some embodiments, the additional therapy is an immune checkpoint inhibitor.

In some embodiments, the immune checkpoint inhibitor is selected from the group consisting of a PD-1 inhibitor, a PD-L1 inhibitor, a CTLA-4 inhibitor, and combinations thereof.

In some embodiments, the immune checkpoint inhibitor is selected from the group consisting of a PD-1 inhibitor, a PD-L1 inhibitor, a CTLA-4 inhibitor, a VISTA inhibitor, a BTLA inhibitor, a TIM-3 inhibitor, a KIR inhibitor, a LAG-3 inhibitor, a TIGIT inhibitor, a CD-96 inhibitor, a SIRPα inhibitor, and combinations thereof.

In some embodiments, the immune checkpoint inhibitor is selected from the group consisting of a PD-1 inhibitor, a PD-L1 inhibitor, a CTLA-4 inhibitor, a LAG-3 (CD223) inhibitor, a TIM-3 inhibitor, a B7-H3 inhibitor, a B7-H4 inhibitor, an A2aR inhibitor, a CD73 inhibitor, a NKG2A inhibitor, a PVRIG/PVRL2 inhibitor, a CEACAM1 inhibitor, a CEACAM 5 inhibitor, a CEACAM 6 inhibitor, a FAK inhibitor, a CCL2 inhibitor, a CCR2 inhibitor, a LIF inhibitor, a CD47 inhibitor, a SIRPα inhibitor, a CSF-1 inhibitor, an M-CSF inhibitor, a CSF-1R inhibitor, an IL-1 inhibitor, an IL-1R3 inhibitor, an IL-RAP inhibitor, an IL-8 inhibitor, a SEMA4D inhibitor, an Ang-2 inhibitor, a CELVER-1 inhibitor, an Axl inhibitor, a phsphatidylserine inhibitor, and combinations thereof.

In some embodiments, the immune checkpoint inhibitor is selected from those shown in Table 10, or combinations thereof.

TABLE 10
Exemplary Immune Checkpoint Inhibitors

| Target | Inhibitor |
|---|---|
| LAG-3 (CD223) | LAG525 (IMP701), REGN3767 (R3767), |
| | BI 754, 091, tebotelimab (MGD013), |
| | eftilagimod alpha (IMP321), |
| | FS118 |
| TIM-3 | MBG453, Sym023, TSR-022 |
| B7-H3, B7-H4 | MGC018, FPA150 |
| A2aR | EOS100850, AB928 |
| CD73 | CPI-006 |
| NKG2A | Monalizumab |
| PVRIG/PVRL2 | COM701 |
| CEACAM1 | CM24 |
| CEACAM 5/6 | NEO-201 |
| FAK | Defactinib |
| CCL2/CCR2 | PF-04136309 |
| LIF | MSC-1 |
| CD47/SIRPα | Hu5F9-G4 (5F9), ALX148, TTI-662, |
| | RRx-001 |
| CSF-1 | Lacnotuzumab (MCS110), LY3022855, |
| (M-CSF)/CSF-1R | SNDX-6352, emactuzumab |
| | (RG7155), pexidartinib (PLX3397) |
| IL-1 and IL-1R3 | CAN04, Canakinumab (ACZ885) |
| (IL-IRAP) | |
| IL-8 | BMS-986253 |
| SEMA4D | Pepinemab (VX15/2503) |
| Ang-2 | Trebananib |
| CLEVER-1 | FP-1305 |
| Axl | Enapotamab vedotin (EnaV) |
| Phosphatidylserine | Bavituximab |

In some embodiments, the immune checkpoint inhibitor is an antibody. In some embodiments, the PD-1 inhibitor is selected from the group consisting of pembrolizumab, nivolumab, toripalimab, cemiplimab-rwlc, sintilimab, and combinations thereof. In some embodiments, the PD-L1 inhibitor is selected from the group consisting of atezolizumab, durvalumab, avelumab, and combinations thereof. In some embodiments, the CTLA-4 inhibitor is ipilimumab. In some embodiments, the PD-1 inhibitor is selected from the group of inhibitors shown in Table 11.

TABLE 11
Exemplary PD-1 Inhibitor Antibodies

| Name | Internal Name | Antigen | Company |
|---|---|---|---|
| nivolumab | Opdivo, ONO-4538, MDX-1106, | PD-1 | BMS, Medarex, Ono |
| | BMS-936558, 5C4 | | |
| pembrolizumab | Keytruda, MK-3475, SCH | PD-1 | Merck (MSD), Schering- |
| | 900475, lambrolizumab | | Plough |
| toripalimab | JS001, JS-001, TAB001, | PD-1 | Junmeng Biosciences, Shanghai |
| | Triprizumab | | Junshi, TopAlliance Bio |
| cemiplimab-rwlc | Libtayo, cemiplimab, | PD-1 | Regeneron, Sanofi |
| | REGN2810 | | |
| sintilimab | Tyvyt, IBI308 | PD-1 | Adimab, Innovent, Lilly |
| MEDI0680 | AMP-514 | PD-1 | Amplimmune, Medimmune |
| LZM009 | | PD-1 | Livzon |
| vudalimab | XmAb20717 | CTLA4, | Xencor |
| | | PD-1 | |
| SI-B003 | | CTLA4, | Sichuan Baili |
| | | PD-1 | Pharma, Systimmune |
| Sym021 | Symphogen patent anti-PD-1 | PD-1 | Symphogen |
| LVGN3616 | | PD-1 | Lyvgen Biopharma |
| MGD019 | | CTLA4, | MacroGenics |
| | | PD-1 | |
| MEDI5752 | | CTLA4, | Medimmune |
| | | PD-1 | |
| CS1003 | | PD-1 | CStone Pharma |
| IBI319 | IBI-319 | PD-1, | Innovent, Lilly |
| | | unknown | |
| IBI315 | IBI-315 | HER2/neu, | Beijing Hanmi, Innovent |
| | | PD-1 | |
| budigalimab | ABBV-181, PR-1648817 | PD-1 | Abbvie |
| Sunshine Guojian | 609A | PD-1 | Sunshine Guojian Pharma |
| patent anti-PD-1 | | | |
| F520 | | PD-1 | Shandong New Time Pharma |
| RO7247669 | | LAG-3, | Roche |
| | | PD-1 | |
| izuralimab | XmAb23104 | ICOS, | Xencor |
| | | PD-1 | |
| LY3434172 | | PD-1, | Lilly, Zymeworks |
| | | PD-L1 | |
| SG001 | | PD-1 | CSPC Pharma |
| QL1706 | PSB205 | CTLA4, | Sound Biologics |
| | | PD-1 | |
| AMG 404 | AMG404 | PD-1 | Amgen |
| MW11 | | PD-1 | Mabwell |
| GNR-051 | | PD-1 | IBC Generium |
| Ningbo Cancer | HerinCAR-PD1 | PD-1 | Ningbo Cancer Hosp. |
| Hosp. anti-PD-1 | | | |
| CAR | | | |
| Chinese PLA | | PD-1 | Chinese PLA Gen. Hosp. |
| Gen. Hosp. | | | |
| anti-PD-1 | | | |
| cetrelimab | JNJ-63723283 | PD-1 | Janssen Biotech |
| TY101 | | PD-1 | Tayu Huaxia |
| AK112 | | PD-1, | Akeso |
| | | VEGF | |
| EMB-02 | | LAG-3, | EpimAb |
| | | PD-1 | |
| pidilizumab | CT-011, hBat-1, MDV9300 | PD-1 | CureTech, Medivation, Teva |
| sasanlimab | PF-06801591, RN-888 | PD-1 | Pfizer |
| balstilimab | AGEN2034, AGEN-2034 | PD-1 | Agenus, Ludwig Inst., Sloan- |
| | | | Kettering |
| geptanolimab | CBT-501, GB226, GB 226, | PD-1 | CBT Pharma, Genor |
| | Genolimzumab, Genormab | | |
| RO7121661 | | PD-1, | Roche |
| | | TIM-3 | |
| AK104 | | CTLA4, | Akeso |
| | | PD-1 | |
| pimivalimab | JTX-4014 | PD-1 | Jounce |
| IBI318 | IBI-318 | PD-1, | Innovent, Lilly |
| | | PD-L1 | |
| BAT1306 | | PD-1 | Bio-Thera Solutions |
| ezabenlimab | BI754091, BI 754091 | PD-1 | Boehringer |
| Henan Cancer | Teripalimab | PD-1 | Henan Cancer Hospital |
| Hospital anti-PD-1 | | | |
| tebotelimab | | LAG-3, | MacroGenics |
| | | PD-1 | |
| sindelizumab | | PD-1 | Nanjing Medical U. |
| dostarlimab | ANB011, TSR-042, ABT1 | PD-1 | AnaptysBio, Tesaro |
| tislelizumab | BGB-A317 | PD-1 | BeiGene, Celgene |
| spartalizumab | PDR001, BAP049 | PD-1 | Dana-Farber, Novartis |
| retifanlimab | MGA012, INCMGA00012 | PD-1 | Incyte, MacroGenics |
| camrelizumab | SHR-1210 | PD-1 | Incyte, Jiangsu |
| | | | Hengrui, Shanghai Hengrui |
| zimberelimab | WBP3055, GLS-010, AB122 | PD-1 | Arcus, Guangzhou Gloria |
| | | | Bio, Harbin Gloria |
| | | | Pharma, WuXi Biologics |
| penpulimab | AK105 | PD-1 | Akeso, HanX Bio, Taizhou |
| | | | Hanzhong Bio |
| prolgolimab | BCD-100 | PD-1 | Biocad |
| HX008 | | PD-1 | Taizhou Hanzhong |
| | | | Bio, Taizhou HoudeAoke Bio |
| SCT-I10A | | PD-1 | Sinocelltech |
| serplulimab | HLX10 | PD-1 | Henlix |

In some embodiments, the PD-L1 inhibitor is selected from the group of inhibitors shown in Table 12.

TABLE 12
Exemplary PD-L1 Inhibitor Antibodies

| Name | Internal Name | Antigen | Company |
|---|---|---|---|
| durvalumab | Imfinzi, MEDI-4736, | PD-L1 | AstraZeneca, Celgene, |
| | MEDI4736 | | Medimmune |
| atezolizumab | Tecentriq, MPDL3280A, | PD-L1 | Genentech |
| | RG7446, YW243.55.S70, | | |
| | RO5541267 | | |
| avelumab | Bavencio, MSB0010718C, | PD-L1 | Merck Serono, Pfizer |
| | A09-246-2 | | |
| AMP-224 | | PD-L1 | Amplimmune, GSK, Medimmune |
| cosibelimab | CK-301, TG-1501 | PD-L1 | Checkpoint Therapeutics, |
| | | | Dana-Farber, Novartis, |
| | | | TG Therapeutics |
| lodapolimab | LY3300054 | PD-L1 | Lilly |
| MCLA-145 | | 4-1BB, | Merus |
| | | PD-L1 | |
| FS118 | | LAG-3, | f-star, Merck Serono |
| | | PD-L1 | |
| INBRX-105 | ES101 | 4-1BB, | Elpiscience, Inhibrx |
| | | PD-L1 | |
| Suzhou | | PD-L1 | Suzhou Nanomab |
| Nanomab patent | | | |
| anti-PD-L1 | | | |
| MSB2311 | | PD-L1 | Mabspace |
| BCD-13 | | PD-L1 | Biocad |
| opucolimab | HLX20, HLX09 | PD-L1 | Henlix |
| IBI322 | IBI-322 | CD47, | Innovent |
| | | PD-L1 | |
| LY3415244 | | PD-L1, | Lilly, Zymeworks |
| | | TIM-3 | |
| GR1405 | | PD-L1 | Genrix Biopharma |
| LY3434172 | | PD-1, | Lilly, Zymeworks |
| | | PD-L1 | |
| CDX-527 | | CD27, | Celldex |
| | | PD-L1 | |
| FS222 | | 4-1BB, | f-star |
| | | PD-L1 | |
| LDP | | PD-L1 | Dragonboat Biopharma |
| ABL503 | | 4-1BB, | ABL Bio |
| | | PD-L1 | |
| HB0025 | | PD-L1, | Huabo Biopharm |
| | | VEGF | |
| MDX-1105 | BMS-936559, 12A4 | PD-L1 | Medarex |
| garivulimab | BGB-A333 | PD-L1 | BeiGene |
| GEN1046 | | 4-1BB, | BioNTech, Genmab |
| | | PD-L1 | |
| NM21-1480 | | 4-1BB, | Numab |
| | | PD-L1, | |
| | | Serum | |
| | | Albumin | |
| bintrafusp alfa | M7824, MSB0011359C | PD-L1, | Merck Serono, NCI |
| | | TGFβRII | |
| pacmilimab | CX-072 | PD-L1 | CytomX |
| A167 | KL-A167 | PD-L1 | Harbour Biomed Ltd., Sichuan |
| | | | Kelun Pharma |
| IBI318 | IBI-318 | PD-1, | Innovent, Lilly |
| | | PD-L1 | |
| KN046 | | CTLA4, | Alphamab |
| | | PD-L1 | |
| STI-3031 | IMC-001 | PD-L1 | Sorrento |
| SHR-1701 | | PD-L1 | Jiangsu Hengrui |
| LP002 | | PD-L1 | Taizhou HoudeAoke Bio |
| STI-1014 | ZKAB001 | PD-L1 | Lee's Pharm, Sorrento |
| envafolimab | KN035 | PD-L1 | Alphamab |
| adebrelimab | SHR-1316 | PD-L1 | Jiangsu Hengrui, Shanghai |
| | | | Hengrui |
| CS1001 | | PD-L1 | CStone Pharma |
| TQB2450 | CBT-502 | PD-L1 | CBT Pharma, Chia Tai Tianqing |
| | | | Pharma |

In some embodiments, the CTLA-4 inhibitor is selected from the group of inhibitors shown in Table 13.

TABLE 13
Exemplary CTLA4 Inhibitor Antibodies

| Name | Internal Name | Antigen | Company |
|---|---|---|---|
| ipilimumab | Yervoy, MDX-010, | CTLA4 | Medarex |
| | MDX101, 10D1, | | |
| | BMS-734016 | | |
| ATOR-1015 | ADC-1015 | CTLA4, OX40 | Alligator |
| vudalimab | XmAb20717 | CTLA4, PD-1 | Xencor |
| SI-B003 | | CTLA4, PD-1 | Sichuan Baili |
| | | | Pharma, Systimmune |
| MGD019 | | CTLA4, PD-1 | MacroGenics |
| MEDI5752 | | CTLA4, PD-1 | Medimmune |
| ADU-1604 | | CTLA4 | Aduro |
| BCD-145 | Q3W | CTLA4 | Biocad |
| CS1002 | | CTLA4 | CStone Pharma |
| REGN4659 | | CTLA4 | Regeneron |
| pavunalimab | XmAb22841 | CTLA4, LAG-3 | Xencor |
| AGEN1181 | | CTLA4 | Agenus |
| QL1706 | PSB205 | CTLA4, PD-1 | Sound Biologics |
| ADG126 | | CTLA4 | Adagene |
| KN044 | | CTLA4 | Changchun Intelli-Crown |
| ONC-392 | | CTLA4 | OncoImmune, Pfizer |
| BMS-986218 | | CTLA4 | BMS |
| BMS-986249 | | CTLA4 | BMS |
| BT-001 | TG6030 | CTLA4 | BioInvent |
| quavonlimab | MK-1308 | CTLA4 | Merck (MSD) |
| zalifrelimab | AGEN1884 | CTLA4 | Agenus, Ludwig Inst., |
| | | | Sloan-Kettering |
| AK104 | | CTLA4, PD-1 | Akeso |
| IBI310 | IBI-310 | CTLA4 | Innovent |
| KN046 | | CTLA4, PD-L1 | Alphamab |
| tremelimumab | ticilimumab, | CTLA4 | Amgen, Medimmune, Pfizer |
| | CP-675206, clone 11.2.1 | | |

In some embodiments, the immune checkpoint inhibitor is a small molecule drug. Small molecule checkpoint inhibitors are described, e.g., in WO2015/034820A1, WO2015/160641A2, WO2018/009505 A1, WO2017/066227 A1, WO2018/044963 A1, WO2018/026971 A1, WO2018/045142 A1, WO2018/005374 A1, WO2017/202275 A1, WO2017/202273 A1, WO2017/202276 A1, WO2018/006795 A1, WO2016/142852 A1, WO2016/142894 A1, WO2015/033301 A1, WO2015/033299 A1, WO2016/142886 A2, WO2016/142833 A1, WO2018/051255 A1, WO2018/051254 A1, WO2017/205464 A1, US2017/0107216 A1, WO2017/070089A1, WO2017/106634A1, US2017/0174679 A1, US2018/0057486 A1, WO2018/013789 A1, US2017/0362253 A1, WO2017/192961 A1, WO2017/118762 A1, US2014/199334 A1, WO2015/036927 A1, US2014/0294898 A1, US2016/0340391 A1, WO2016/039749 A1, WO2017/176608 A1, WO2016/077518 A1, WO2016/100608 A1, US2017/0252432 A1, WO2016/126646 A1, WO2015/044900 A1, US2015/0125491 A1, WO2015/033303 A1, WO2016/142835 A1, WO2019/008154 A1, WO2019/008152 A1, and WO2019023575A1.

In some embodiments, the PD-1 inhibitor is 2-[[4-amino-1-[5-(1-amino-2-hydroxypropyl)-1,3,4-oxadiazol-2-yl]-4-oxobutyl]carbamoylamino]-3-hydroxypropanoic acid (CA-170).

In some embodiments, the immune checkpoint inhibitor is (S)-1-(3-Bromo-4-((2-bromo-[1,1′-biphenyl]-3-yl)methoxy)benzyl)piperidine-2-carboxylic Acid.

In some embodiments, the immune checkpoint inhibitor is a peptide. See, e.g., Sasikumar et al., “Peptide and Peptide-Inspired Checkpoint Inhibitors: Protein Fragments to Cancer Immunotherapy,” Medicine in Drug Discovery 8:100073 (2020).

### VI. VARIANTS

In some embodiments, the fusion protein(s) or components thereof described herein, or the NK cell genotypes described herein, are at least 80%, e.g., at least 85%, 90%, 95%, 98%, or 100% identical to the amino acid sequence of an exemplary sequence (e.g., as provided herein), e.g., have differences at up to 1%, 2%, 5%, 10%, 15%, or 20% of the residues of the exemplary sequence replaced, e.g., with conservative mutations, e.g., including or in addition to the mutations described herein. In preferred embodiments, the variant retains desired activity of the parent.

To determine the percent identity of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions are then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

Percent identity between a subject polypeptide or nucleic acid sequence (i.e. a query) and a second polypeptide or nucleic acid sequence (i.e. target) is determined in various ways that are within the skill in the art, for instance, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed, pp 353-358; BLAST program (Basic Local Alignment Search Tool; (Altschul, S. F., W. Gish, et al. (1990) J Mol Biol 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for target proteins or nucleic acids, the length of comparison can be any length, up to and including full length of the target (e.g., 5%, 10%, 20%, 30%, 40%, 50%, 60%, 70%, 80%, 90%, 95%, or 100%). For the purposes of the present disclosure, percent identity is relative to the full length of the query sequence.

For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5.

Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein.

The term “in vivo” is used to describe an event that takes place in a subject's body. The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay. The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the term “buffer solution” refers to an aqueous solution consisting of a mixture of a weak acid and its conjugate base, or vice versa. As used herein, the term “cell culture medium” refers to a mixture for growth and proliferation of cells in vitro, which contains essential elements for growth and proliferation of cells such as sugars, amino acids, various nutrients, inorganic substances, etc. A buffer solution, as used herein, is not a cell culture medium.

As used herein, the term “bioreactor” refers to a culture apparatus capable of continuously controlling a series of conditions that affect cell culture, such as dissolved oxygen concentration, dissolved carbon dioxide concentration, pH, and temperature.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Some vectors are suitable for delivering the nucleic acid molecule(s) or polynucleotide(s) of the present application. Certain vectors are capable of directing the expression of nucleic acids to which they are operatively linked. Such vectors are referred to herein as expression vectors.

The term “operably linked” refers to two or more nucleic acid sequence or polypeptide elements that are usually physically linked and are in a functional relationship with each other. For instance, a promoter is operably linked to a coding sequence if the promoter is able to initiate or regulate the transcription or expression of a coding sequence, in which case, the coding sequence should be understood as being “under the control of” the promoter.

The terms “host cell,” “host cell line,” and “host cell culture” are used interchangeably and refer to cells into which exogenous nucleic acid has been introduced, including the progeny of such cells. Host cells include “engineered cells,” “transformants,” and “transformed cells,” which include the primary engineered (e.g., transformed) cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As appropriate, the host cells can be stably or transiently transfected with a polynucleotide encoding a fusion protein, as described herein.

The section headings used herein are for organizational purposes only and are not to be construed as limiting the subject matter described.

### VIII. EXAMPLES

The following examples are included for illustrative purposes only and are not intended to limit the scope of the invention.

### Example 1: Off-the-Shelf NK Cell Therapy Platform

One example of a method by which NK cells were expanded and stimulated is shown FIG. 1. A single unit of FDA-licensed, frozen cord blood that has a high affinity variant of the receptor CD16 (the 158 V/V variant, see, e.g., Koene et al., “FcγRIIIa-158V/F Polymorphism Influences the Binding of IgG by Natural Killer Cell FcgammaRIIIa, Independently of the FcgammaRIIIa-48L/R/H Phenotype,” Blood 90:1109-14 (1997).) and the KIR-B genotype (KIR B allele of the KIR receptor family, see, e.g., Hsu et al., “The Killer Cell Immunoglobulin-Like Receptor (KIR) Genomic Region: Gene-Order, Haplotypes and Allelic Polymorphism,” Immunological Review 190:40-52 (2002); and Pyo et al., “Different Patterns of Evolution in the Centromeric and Telomeric Regions of Group A and B Haplotypes of the Human Killer Cell Ig-like Receptor Locus,” PLoS One 5:e15115 (2010)) was selected as the source ofNK cells.

The cord blood unit was thawed and the freezing medium was removed via centrifugation. The cell preparation was then depleted of T cells using the QuadroMACS Cell Selection System (Miltenyi) and CD3 (T cell) MicroBeads. A population of 6×10⁸ total nucleated cells (TNC) were labelled with the MicroBeads and separated using the QuadroMACS device and buffer. Following depletion of T cells, the remaining cells, which were predominantly monocytes and NK cells, were washed and collected in antibiotic-free medium (CellgroSCGM). The cell preparation was then evaluated for total nucleated cell count, viability, and % CD3+ cells. As shown in FIG. 1, the cord blood NK cells were CD3 depleted.

The CD3− cell preparation was inoculated into a gas permeable cell expansion bag containing growth medium. The cells were co-cultured with replication incompetent engineered HuT-78 (eHUT-78) feeder cells to enhance expansion for master cell bank (MCB) production. The CellgroSCGM growth media was initially supplemented with anti-CD3 antibody (OKT3), human plasma, glutamine, and IL-2. As shown in FIG. 1, the NK cells are optionally engineered, e.g., to introduce CARs into the NK cells, e.g., with a lentiviral vector, during one of the co-culturing steps.

The cells were incubated as a static culture for 12-16 days at 37° C. in a 5% CO₂ balanced air environment, with additional exchanges of media occurring every 2 to 4 days. After the culture expanded more than 100-fold, the cultured cells were harvested and then suspended in freezing medium and filled into cryobags. In this example, 80 bags or vials at 10⁸ cells per bag or vial were produced during the co-culture. The cryobags were frozen using a controlled rate freezer and stored in vapor phase liquid nitrogen (LN₂) tanks below −150° C. These cryopreserved NK cells derived from the FDA-licensed cord blood unit served as the master cell bank (MCB).

To produce the drug product, a bag of frozen cells from the MCB was thawed and the freezing medium was removed. The thawed cells were inoculated into a disposable culture bag and co-cultured with feeder cells, e.g., eHUT78 feeder cells to produce the drug product. In this example, the cells are cultured in a 50 L bioreactor to produce thousands of lots of the drug product per unit of cord blood (e.g., 4,000-8,000 cryovials at 10⁹ cells/vial), which are mixed with a cryopreservation composition and frozen in a plurality of storage vessels such as cryovials. The drug product is an off-the-shelf infusion ready product that can be used for direct infusion. Each lot of the drug product can be used to infuse hundreds to thousands of patients (e.g., 100-1,000 patients, e.g. with a target dose of 4×10⁹ cells).

### Example 2: Feeder Cell Expansion

As one example, suitable feeder cells, e.g., eHut-78 cells, were thawed from a frozen stock and expanded and cultured in a 125 mL flask in growth medium comprising RPMI1640 (Life Technologies) 89% v/v, inactivated fetal bovine serum (FBS) (Life Technologies) (10% v/v), and glutamine (hyclone) (2 mM) at or at about 37° C. and at or at about 3-7% CO₂ for or for about 18-24 days. The cells were split every 2-3 days into 125 mL-2 L flasks. The cells were harvested by centrifugation and gamma irradiated. The harvested and irradiated cells were mixed with a cryopreservation medium (Cryostor CS10) in 2 mL cryovials and frozen in a controlled rate freezer, with a decrease in temperature of about 15° C. every 5 minutes to a final temperature of or of about −90° C., after which they were transferred to a liquid nitrogen tank or freezer to a final temperature of or of about −150° C. After freezing, cell viability was greater than or equal to 70% of the original number of cells (here, at least 1.0×10⁸ viable cells/mL), and 85% or more of the cells expressed mTNF-α, 85% or more of the cells expressed mbIL-21+, and 85% or more of the cells expressed 4-1BBL.

### Example 3: NK Cell Expansion and Stimulation

As one example, suitable NK cells can be prepared as follows using HuT-78 cells transduced to express 4-1BBL, membrane bound IL-21 and mutant TNFalpha (“eHut-78P cells”) as feeder cells. The feeder cells are suspended in 1% (v/v) CellGro medium and are irradiated with 20,000 cGy in a gamma-ray irradiator. Seed cells (e.g., CD3-depleted PBMC or CD3-depleted cord blood cells) are grown on the feeder cells in CellGro medium containing human plasma, glutamine, L-2, OKT-3 in static culture at 37° C. The cells are split every 2-4 days. The total culture time was 19 days. The NK cells are harvested by centrifugation and cryopreserved. Thawed NK are administered to patients in infusion medium consisting of: Phosphate Buffered Saline (PBS 1×, FujiFilm Irvine) (50% v/v), albumin (human) (20% v/v of OctaPharma albumin solution containing: 200 g/L protein, of which ≥96% is human albumin, 130-160 mmol sodium; <2 mmol potassium, 0.064-0.096 mmol/g protein N-acetyl-DL-tryptophan, 0.064-0.096 mmol/g protein, caprylic acid, ad. 1000 ml water), Dextran 40 in Dextrose (25% v/v of Hospira Dextran 40 in Dextrose Injection, USP containing: 10 g/100 mL Dextran 40 and 5 g/100 mL dextrose hydrous in water) and dimethyl sulfoxide (DMSO) (5% v/v of Avantor DMSL solution with a density of 1.101 g/cm³ at 20° C.).

In some case, the seed cells are CD3-depleted cord blood cells. A cell fraction can be depleted of CD3 cells by immunomagnetic selection, for example, using a CliniMACS T cell depletion set ((LS Depletion set (162-01) Miltenyi Biotec). Preferably, the cord blood seed cells are selected to express CD16 having the V/V polymorphism at F158 (Fc gamma RIIIa-158 V/V genotype) (Musolino et al. 2008 J Clin Oncol 26:1789). Preferably, the cord blood seed cells are KIR-B haplotype.

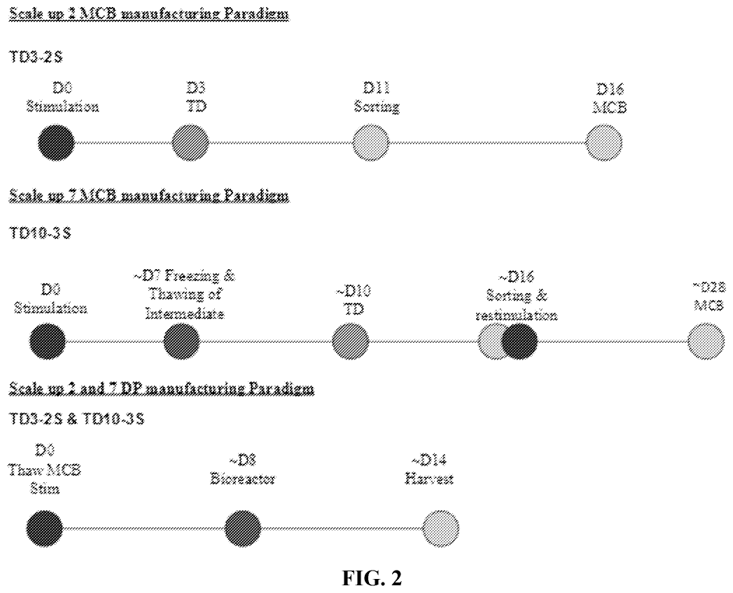

Examples of two different manufacturing timelines are shown in FIG. 2. In one scheme, a master cell bank (MCB) is generated by stimulation of a NK cell source (e.g., a single cord blood unit) with feeder cells (e.g., eHuT-78, as described herein) starting at day 0 (DO), followed by transduction, e.g., with a vector comprising a CAR described herein, e.g., as described in Example 6, at Day 3 (D3), followed by sorting, e.g., for CAR expression, at day 11 (D11), and harvesting and cryopreserving for a MCB at day 16 (D16). In another scheme, a MCB is generated by stimulation of a NK cell source (e.g., a single cord blood unit) with feeder cells (e.g., eHuT-78, as described herein) starting at day 9 (DO), followed by freezing & thawing of an intermediate at around day 7 (D7), transduction, e.g., with a vector comprising a CAR described herein, at around day 10 (D10), sorting and restimulation at around day 16 (D16) and harvesting for a MCB at about day 28 (D28). In some cases, a drug product (DP) is manufactured by thawing and stimulating a MCB (e.g., derived from one of the manufacturing timelines described above) with feeder cells (e.g., eHuT-78, as described here), starting at day 0 (DO), followed by bioreactor culturing at about day 8 (D8) and harvesting and cryopreserving for a drug DP at about day 14.In these examples, the initial NK:feeder cell ratio can be 1:2.5 and incubation can occur, for example as static culture at 37° C. in a 5% CO₂ balanced air environment in a growth medium (for example, those described herein). Sorting can be carried out, for example, using an antibody specific for the CAR. The resulting cells can be frozen in a cryopreservation medium (for example, as described herein).

### Example 4: Cord Blood as an NK Cell Source

NK cells make up five to 15% of peripheral blood lymphocytes. Traditionally, peripheral blood has been used as the source for NK cells for therapeutic use. However, as shown herein, NK cells derived from cord blood have a nearly ten-fold greater potential for expansion in the culture systems described herein than those derived from peripheral blood, without premature exhaustion or senescence of the cells. The expression of receptors of interest on the surface of NK cells, such as those involved in the activation of NK cells on engagement of tumor cells, was seen to be more consistent donor-to-donor for cord blood NKs than peripheral-blood NK cells. The use of the manufacturing process described herein consistently activated the NK cells in cord blood in a donor-independent manner, resulting in a highly scaled, active and consistent NK cell product.

### Example 5: Expanded and Stimulated NK-Cell Phenotype

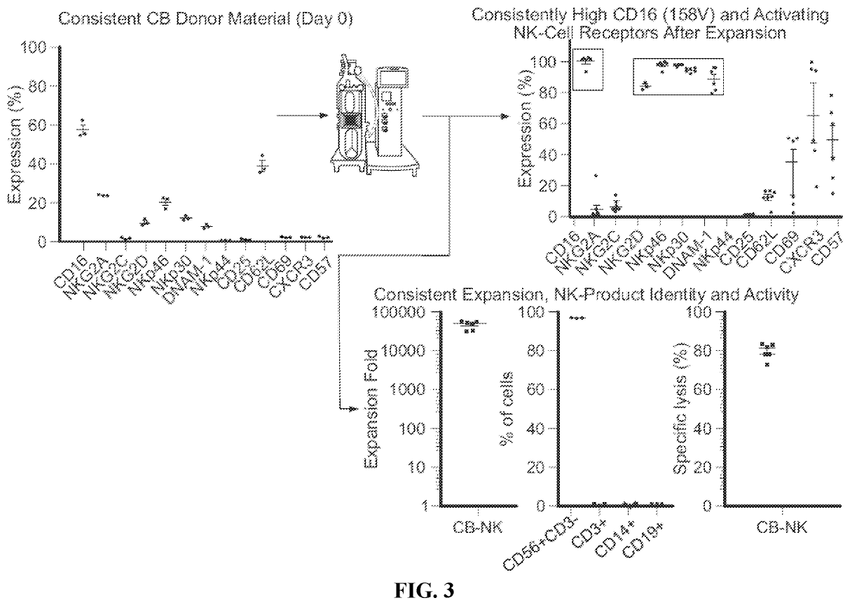

In one example, NK cells from a cord blood unit are expanded and stimulated with eHut-78 cells, according to the expansion and stimulation process described in Example 1. As shown in FIG. 3, the resulting expanded and stimulated population of NK cells have consistently high CD16 (158V) and activating NK-cell receptor expression.

### Example 6: CAR-NK Production (AB-202)

AB-202 is an off-the-shelf allogenic anti-CD19 CAR-NK manufactured from cord blood. AB-202 expresses an anti-CD19 CAR comprising SEQ ID NO: 70 and IL-15 comprising SEQ ID NO: 22. The full expression cassette (SEQ ID NO: 48) includes a signal sequence and T2A. Purity and phenotype of AB-202 was evaluated by flow cytometry. NK cell maturation was determined through expression of the markers, CD56 and CD16, while NK cell activity and regulation are conferred through a balance of activating and inhibitory receptor expression. The expression pattern of these receptors was determined by flow cytometry using receptor-specific reagent antibody staining.

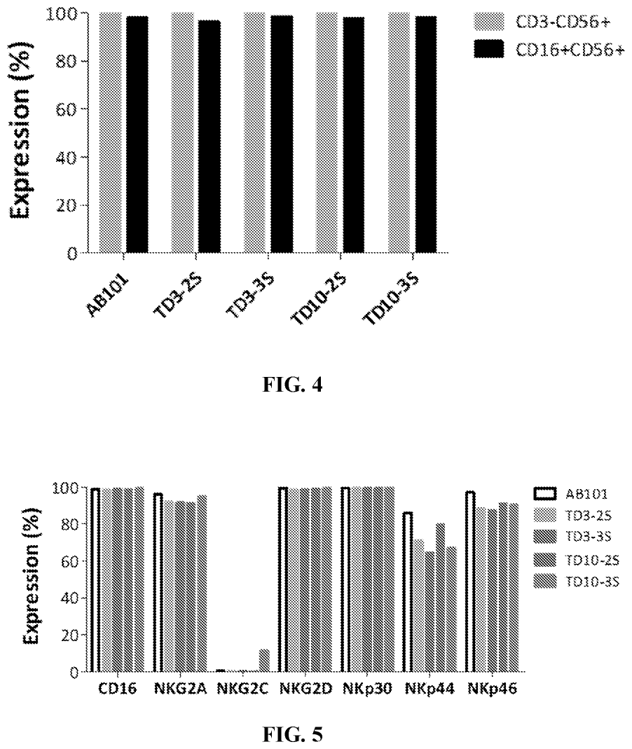

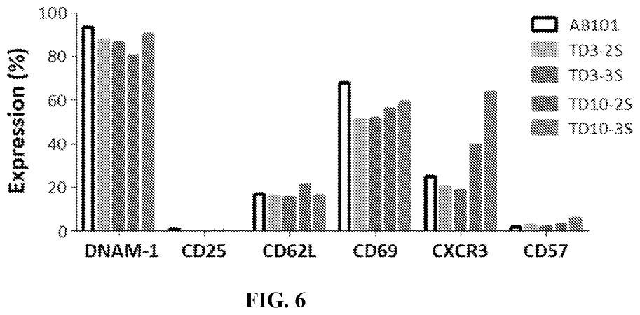

AB-202 purity and identity was determined through the assessment of surface markers CD56. CD56 is the archetypal phenotypic marker of natural killer maturation. Expression of CD16 (FcgRIII) is also an indicator of the NK cell maturation state. AB-202 from the four manufacturing schemes was similar to the non-engineered cord blood-derived NK cells (AB-101) (FIG. 4). Further characterization of AB-202 demonstrated high expression of activating receptors such as NKG2D, NKp30, NKp46, and DNAM-1 and expression of the chemokine receptor, CXCR3 (FIG. 5, FIG. 6). These activating receptors had similar expression levels across the 4 DPs. The chemokine receptor, CXCR3, which is critical for NK cell infiltration into some solid tumors, was differentially expressed with DP from the TD10-3S having the highest expression compared to the other drug production methods and AB-101.

### Example 7: AB-202 in Vitro Cytotoxicity

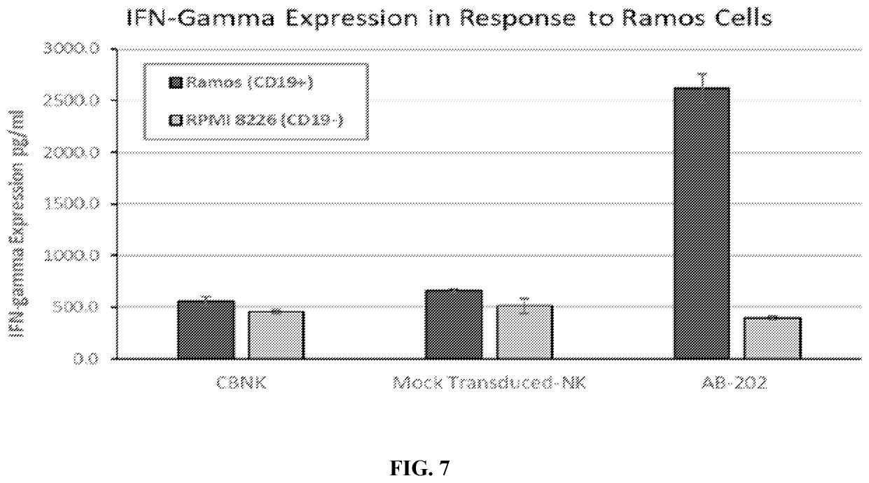

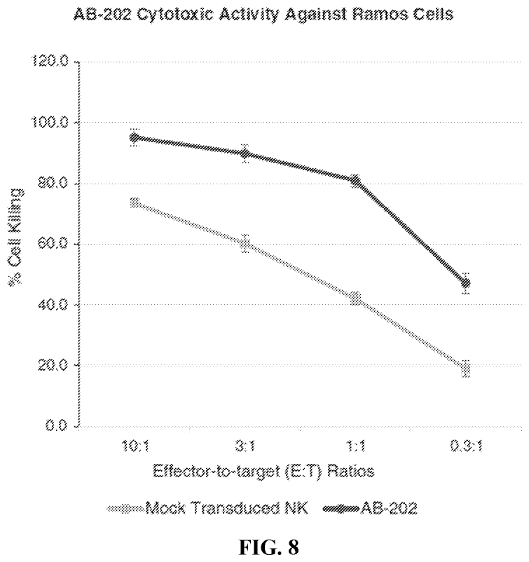

AB-202 displays significant cytotoxic activity against CD19+ target cells (Ramos). As shown in FIG. 7, IFN-gamma secretion from AB-202 cells was increased when exposed to CD19+ Ramos tumor cells. As shown in FIG. 8, AB-202 demonstrated greater activation and cytotoxic activity against CD19+ Ramos cell than non-CAR NK cells.

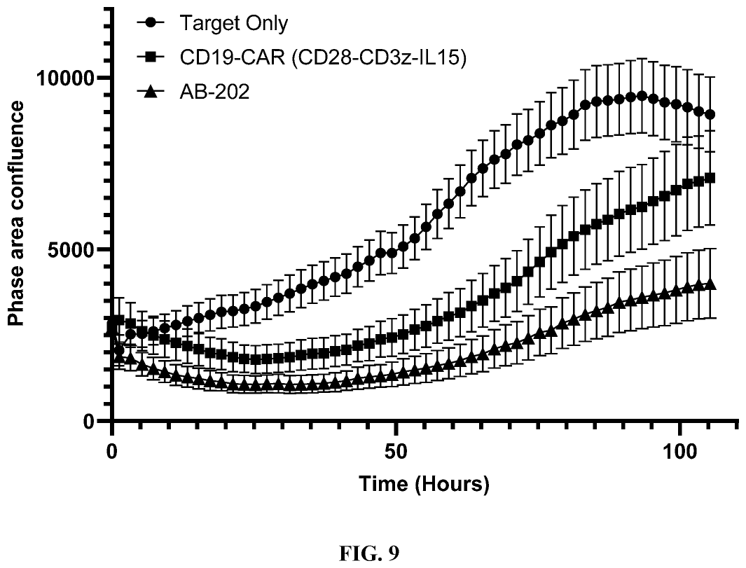

As shown in FIG. 9, AB-202 displays significantly greater killing of CD19+ target cells than the CD-19 CAR-NKs based on Liu et al., “Use of CAR-Transduced Natural Killer Cells in CD19-Positive Lymphoid Tumors,” N Engl J Med 382:545-53 (2020). The AB-202 CAR (SEQ ID NO: 70) comprises an anti-CD19 scFv based on the FMC63 clone (SEQ ID NO: 30), an IgG1 hinge, an IgG1 spacer, a CD28 transmembrane domain, a CD28 costimulatory domain, an OX40L costimulatory domain, and a CD3ζ costimulatory domain. The NK cells expressing the AB-202 CAR were compared to NK cells expressing CD19-CAR CD28-CD3z-IL15 (SEQ ID NO: 73), which includes the same FMC63 clone (SEQ ID NO: 30), IgG1 hinge, IgG1 spacer, CD28 transmembrane domain, CD28 costimulatory domain, and CD3ζ costimulatory domain. Notably, the CD19-CAR CD28-CD3z-IL15 lacks the OX40L costimulatory domain.

The NK cells were incubated with Raji B-cell lymphoma cells at an E:T ratio of 0.3:1 for more than four days. The growth of Raji cells was monitored by measuring phase area confluence using Incucyte® live cell imaging of the cultures. Both AB-202 and the NK cells expressing CD19-CAR CD28-CD3z-IL15 inhibited Raji cell growth compared to Raji cells grown in the absence of NK cells (FIG. 9). AB-202 cells, however, inhibited significantly more growth and was more cytotoxic than NK cells expressing CD19-CAR CD28-CD3z-IL15. The results show that the use of a CAR costimulatory structure that includes the intracellular signaling portion of OX40L improves the cell killing activity of NK cells.

### Example 8: AB-202 Therapy

Patients with advanced heme malignancies with progression beyond all approved therapies (CD19+ relapsed or refractory NHL) are selected for treatment with either 1×10⁸ or 1×10⁹ AB-202 cells.

Lymphodepletion (by administration of both cyclophosmamide and fludarabine) is administered only prior to the first dose of AB-201 (days −5 through −2).

The first dose of AB-202 is administered on day 1. Optional second and third doses are administered on days 29 and 57 or later, e.g., months 2, 3, and 5. Rituximab may be given with additional doses of AB-202 at months 2, 3, and 5.

### Example 9: In Vitro ADCC Activity of AB-202 and Anti-CD20 Antibodies

Thawed CBNK or AB-202 (DP) was cultured with ARH-77 B lymphoblast tumor cells and doses of anti-CD20 antibodies (Rituximab or Obinutuzumab) in a 4 h caspase assay. The CBNK were a non-engineered control lot derived from the same cord blood donor. The CBNK cells were expanded using a similar method as the donor-matched AB-202 except that the CBNK cells did not undergo a lentiviral transduction step. Briefly, the CBNK cells were produced by depleting the cord blood of CD3+ cells. The cells were stimulated with eHuT-78 feeder cells on day 0 and allowed to expand until day 7, at which point the cells were frozen. The cells were thawed and restimulated with eHuT-78 feeder cells. A third stimulation with eHuT-78 feeder cells took place on day 15. The expanded CBNK cells were harvested on day 27.

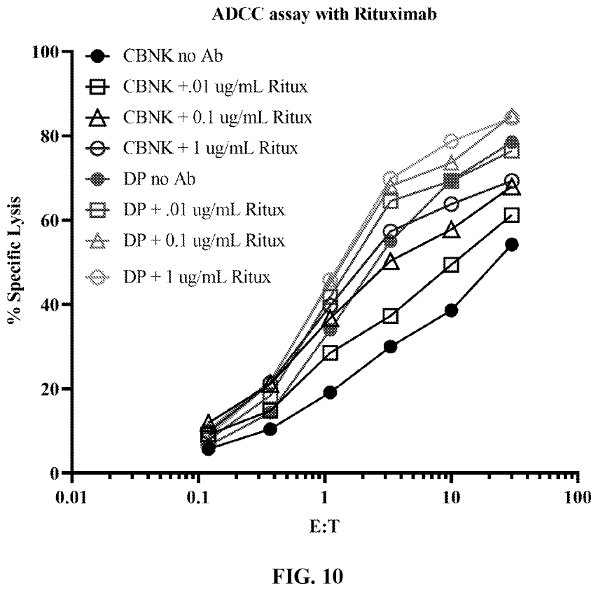

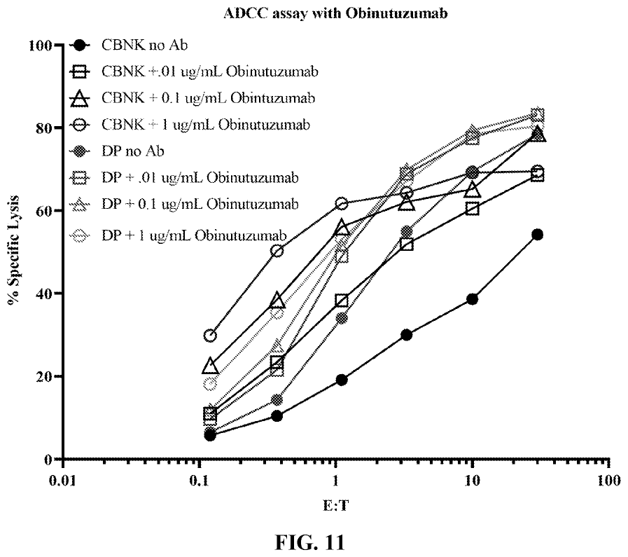

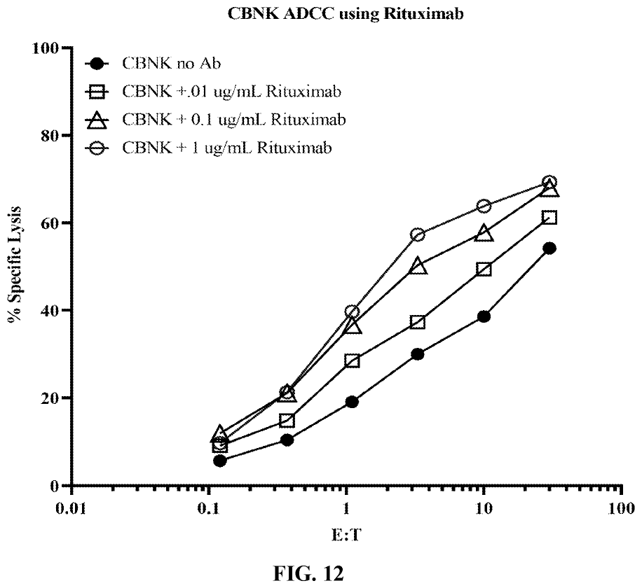

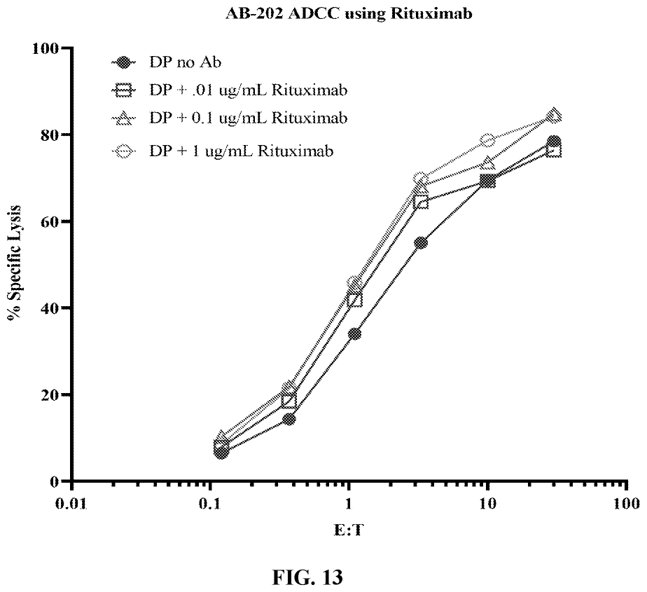

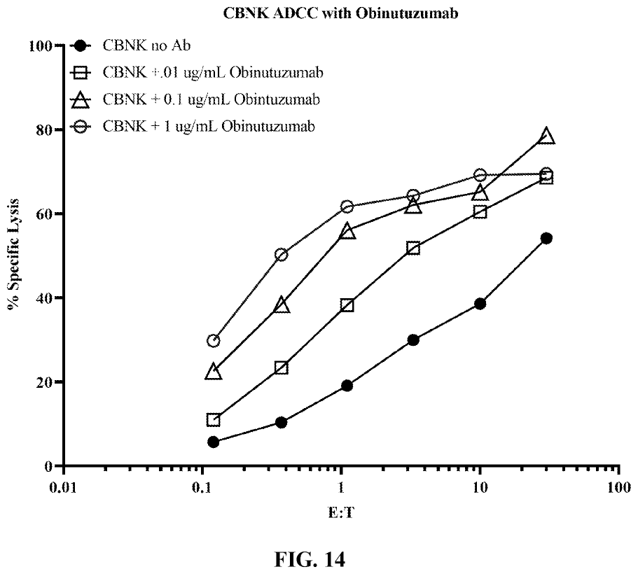

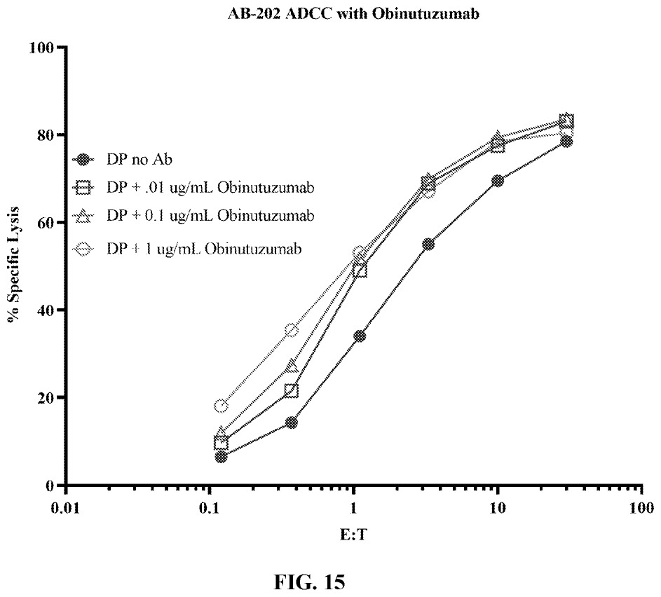

The percent specific lysis is shown for E:T range of 0.12:1 to 30:1 (FIG. 10, FIG. 11, FIG. 12, FIG. 13, FIG. 14, FIG. 15, Table 14).

TABLE 14
Percent Specific Lysis

| | EC:50 |
|---|---|
| CBNK, no antibody | 24.6 |
| CBNK + 0.01 μg/mL Rituximab | 11.0 |
| CBNK + 0.1 μg/mL Rituximab | 3.25 |
| CBNK + 1 μg/mL Rituximab | 2.39 |
| CBNK + 0.01 μg/mL Obinutuzumab | 2.99 |
| CBNK + 0.1 μg/mL Obinutuzumab | 0.85 |
| CBNK + 1 μg/mL Obinutuzumab | 0.37 |
| AB-202, no antibody | 2.8 |
| AB-202 + 0.01 μg/mL Rituximab | 1.9 |
| AB-202 + 0.1 μg/mL Rituximab | 1.56 |
| AB-202 + 1 μg/mL Rituximab | 1.49 |
| AB-202 + 0.01 μg/mL Obinutuzumab | 1.21 |
| AB-202 + 0.1 μg/mL Obinutuzumab | 1.05 |
| AB-202 + 1 μg/mL Obinutuzumab | 0.97 |

Anti-CD20 antibodies induced a dose-dependent increase in ADCC activity. AB-202 showed 8.8-fold higher killing of ARH-77 tumor cells than CBNK in the absence of antibody (EC50 of 2.8 v. 24.6). AB2-202 in the presence of anti-CD20 antibody showed higher killing than AB-202 in the absence of antibody. AB-202 killing activity was increased ˜2-3 fold1.9-fold and 2.9-fold in the presence of 1 μg/mL Rituximab and Obinutuzumab, respectively. in the presence of antibody. CBNK killing activity was increased 10.3-fold with 1 ug/mL Rituximab and 66.5-fold with 1 μg/mL Obinutuzumab versus CBNK, no antibody condition.

### Example 10: Degranulation

NK cells (non-engineered, eHuT-78-expanded cord blood NK cells (CBNK) and AB-202) were cultured in medium alone or with various B cell tumor lines. The CBNK cells were expanded according to the method of Example 10. Data shows the percentage of CD56+ cells that expressed membrane CD107a (LAMP-1), a marker of immune cell activation and cytotoxic degranulation. Data is representative of 4 experiments.

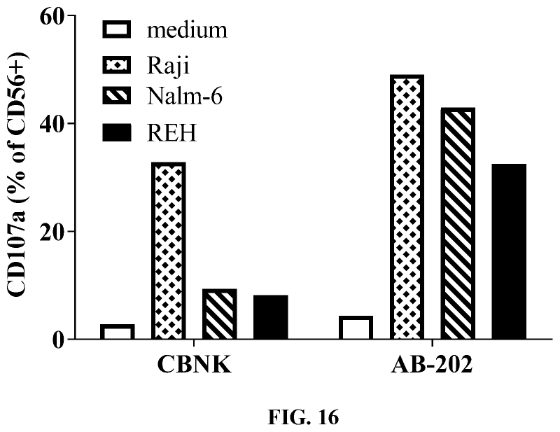

Degranulation of AB-202 cells in response to tumor cell lines was assessed by flow cytometry four hours after stimulation. As shown in FIG. 16, AB-202 displays greater degranulation than non-engineered cord blood NK cells (CBNK).

### Example 11: Intracellular Cyotkine Secretion

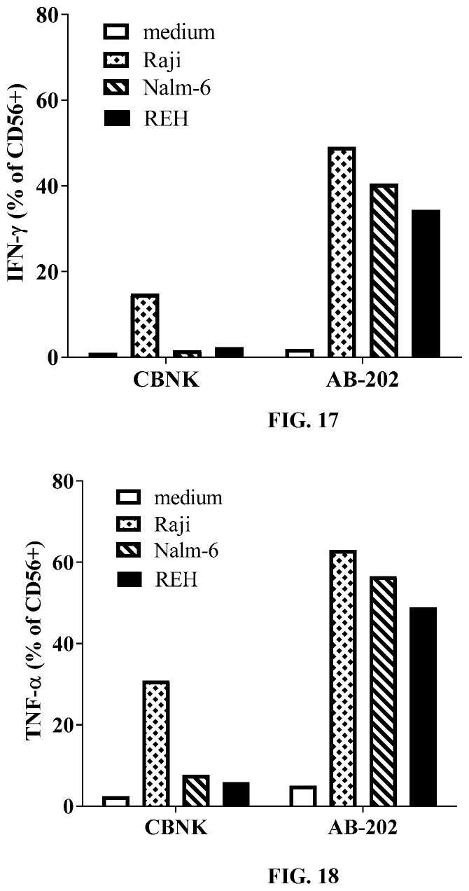

Cytokine production of AB-202 in response to various CD19+ B cell tumor lines was assessed by intracellular flow cytometry four hours after stimulation. As shown in FIG. 17, FIG. 18, and FIG. 19, AB-202 produces cytokines in response to stimulation by various CD19+ tumor cell lines.

### Example 12: In Vivo Efficacy

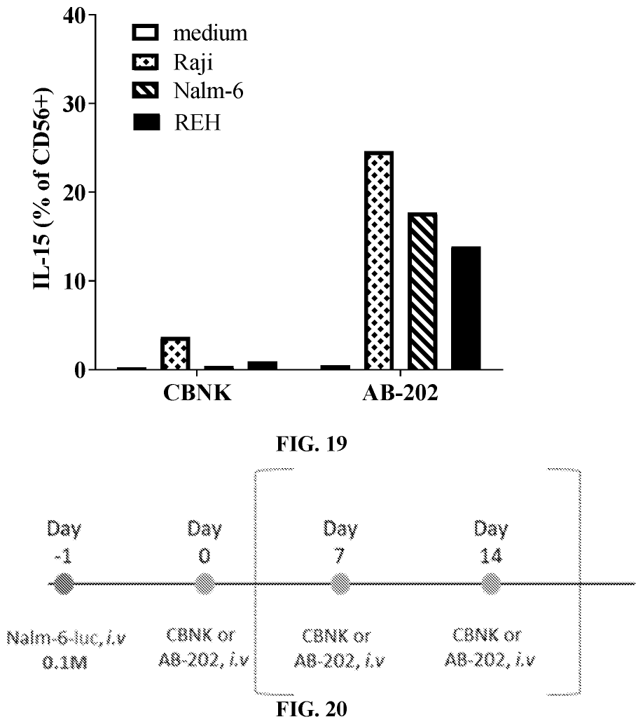

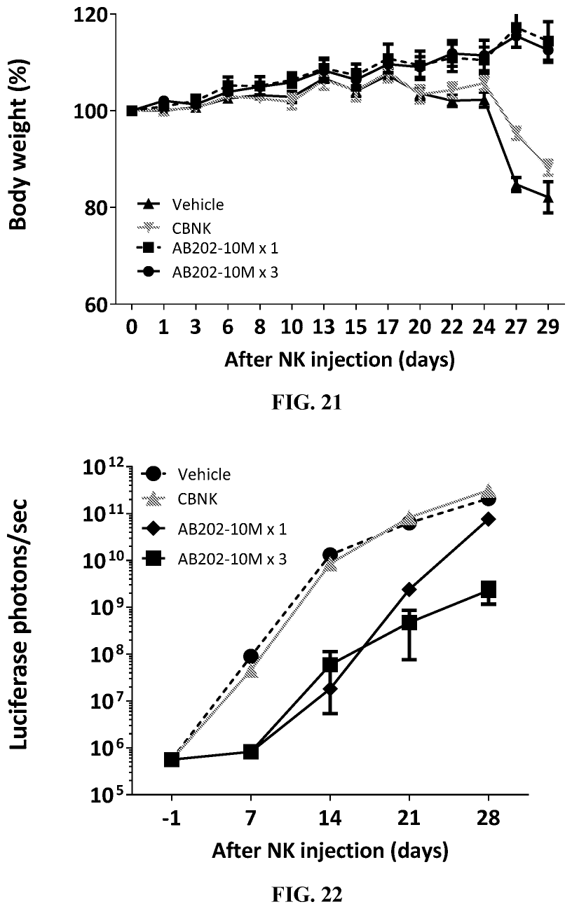

In vivo efficacy of AB-202 in a Nalm-6 model in NSG mice was carried out as shown in FIG. 20. NSG mice were injected intraveneously with 1×10⁵ luciferized Nalm-6 tumor cells. AB-202 was administered intravenously either once or three times beginning one day after tumor cell injection. Non-engineered, eHuT-78 expanded CBNK or vehicle was administered three times beginning one day after tumor cell injection. AB-202 efficacy is shown by reduction in tumor burden on days 7-28. AB-202 treated mice did not lose weight (FIG. 21). On day 28, the luciferase signal of the AB-202-injected groups was lesser than that of the vehicle and CBNK-injected groups, as shown in FIG. 22. On day 29, 2 and 1 out of 5 mice had died in the vehicle and CBNK injected groups, respectively. As shown in FIG. 22, the anti-tumor activity seems to be dose- and injection frequency-dependent. As shown in FIG. 22, AB-202 shows in vivo efficacy in the NSG xenograft model.

### Sequences

| SEQ ID NO: and | |
|---|---|
| DESCRIPTION | SEQUENCE |
| SEQ ID NO: 1 | MEYASDASLDPEAPWPPAPRARACRVLPWALVAGLLLLLLLAAACAVFLACPWAVSGARA |
| Sequence of 4- | SPGSAASPRLREGPELSPDDPAGLLDLRQGMFAQLVAQNVLLIDGPLSWYSDPGLAGVSL |
| 1BBL that can | TGGLSYKEDTKELVVAKAGVYYVFFQLELRRVVAGEGSGSVSLALHLQPLRSAAGAAALA |
| be expressed | LTVDLPPASSEARNSAFGFQGRLLHLSAGQRLGVHLHTEARARHAWQLTQGATVLGLFRV |
| by feeder | TPEIPAGLPSPRSE |
| cells | |
| SEQ ID NO: 2 | MALPVTALLLPLALLLHAARPQDRHMIRMRQLIDIVDQLKNYVNDLVPEFLPAPEDVETN |
| Sequence of | CEWSAFSCFQKAQLKSANTGNNERIINVSIKKLKRKPPSTNAGRRQKHRLTCPSCDSYEK |
| a membrane | KPPKEFLERFKSLLQKMIHQHLSSRTHGSEDSAKPTTTPAPRPPTPAPTIASQPLSLRPE |
| bound IL-21 | ACRPAAGGAVHTRGLDFACDIYIWAPLAGTCGVLLLSLVITLY |
| (mbIL-21) | |
| that can be | |
| expressed by | |
| feeder cells | |
| SEQ ID NO: 3 | MSTESMIRDVELAEEALPKKTGGPQGSRRCLFLSLFSFLIVAGATTLFCLLHFGVIGPQR |
| Sequence of a | EEFPRDLSLISPLAQPVRSSSRTPSDKPVAHVVANPQAEGQLQWLNRRANALLANGVELR |
| mutated TNF | DNQLVVPSEGLYLIYSQVLFKGQGCPSTHVLLTHTISRIAVSYQTKVNLLSAIKSPCQRE |
| alpha (mTNF-a) | TPEGAEAKPWYEPIYLGGVFQLEKGDRLSAEINRPDYLDFAESGQVYFGIIAL |
| that can be | |
| expressed by | |
| feeder cells | |
| SEQ ID NO: 4 | MERVQPLEENVGNAARPRFERNKLLLVASVIQGLGLLLCFTYICLHFSALQVSHRYPRIQ |
| Sequence of | SIKVQFTEYKKEKGFILTSQKEDEIMKVQNNSVIINCDGFYLISLKGYFSQEVNISLHYQ |
| OX40L that | KDEEPLFQLKKVRSVNSLMVASLTYKDKVYLNVTTDNTSLDDFHVNGGELILIHQNPGEF |
| can be | CVL |
| expressed by | |
| feeder cells | |
| SEQ ID NO: 5 | RSKRSRLLHSDYMNMTPRRPGPTRKHYQPYAPPRDFAAYRS |
| CD28 | |
| intracellular | |
| signaling | |
| domain | |
| SEQ ID NO: 6 | AGGAGTAAGAGGAGCAGGCTCCTGCACAGTGACTACATGAACATGACTCCCCGCCGCCCC |
| CD28 | GGGCCCACCCGCAAGCATTACCAGCCCTATGCCCCACCACGCGACTTCGCAGCCTATCGC |
| intracellular | TCC |
| signaling | |
| domain | |
| SEQ ID NO: 7 | CGGAGCAAGAGGTCCCGCCTGCTGCACAGCGACTATATGAACATGACCCCACGGAGACCC |
| Codon | GGCCCTACACGGAAACATTACCAGCCCTATGCTCCACCCCGGGACTTCGCAGCTTACAGA |
| Optimized | AGT |
| CD28 | |
| intracellular | |
| signaling | |
| domain | |
| SEQ ID NO: 8 | ERVQPLEENVGNAARPRFERNK |
| OX40L | |
| intracellular | |
| signaling | |
| domain | |
| SEQ ID NO: 9 | LEENVGNAARPRFERNK |
| OX40L | |
| intracellular | |
| signaling | |
| domain | |
| functional | |
| domain | |
| SEQ ID NO: 10 | RPRFERNK |
| OX40L | |
| intracellular | |
| signaling | |
| domain | |
| functional | |
| domain | |
| SEQ ID NO: 11 | GAAAGGGTCCAACCCCTGGAAGAGAATGTGGGAAATGCAGCCAGGCCAAGATTCGAGAGG |
| OX40L | AACAAG |
| intracellular | |
| signaling | |
| domain | |
| SEQ ID NO: 12 | GAAAGAGTGCAGCCCCTGGAAGAGAATGTCGGGAATGCCGCTCGCCCAAGATTTGAAAGG |
| Codon | AACAAA |
| optimized | |
| OX40L | |
| intracellular | |
| signaling | |
| domain | |
| SEQ ID NO: 13 | RVKFSRSADAPAYQQGQNQLYNELNLGRREEYDVLDKRRGRDPEMGGKPRRKNPQEGLYN |
| CD3ζ signaling | ELQKDKMAEAYSEIGMKGERRRGKGHDGLYQGLSTATKDTYDALHMQALPPR |
| domain | |
| SEQ ID NO: 14 | AGAGTGAAGTTCAGCAGGAGCGCAGACGCCCCCGCGTACCAGCAGGGCCAGAACCAGCTC |
| CD3ζ signaling | TATAACGAGCTCAATCTAGGACGAAGAGAGGAGTACGATGTTTTGGACAAGAGACGTGGC |
| domain | CGGGACCCTGAGATGGGGGGAAAGCCGAGAAGGAAGAACCCTCAGGAAGGCCTGTACAAT |
| | GAACTGCAGAAAGATAAGATGGCGGAGGCCTACAGTGAGATTGGGATGAAAGGCGAGCGC |
| | CGGAGGGGCAAGGGGCACGATGGCCTTTACCAGGGTCTCAGTACAGCCACCAAGGACACC |
| | TACGACGCCCTTCACATGCAGGCCCTGCCCCCTCGC |
| SEQ ID NO: 15 | CGAGTGAAGTTCAGCAGGTCCGCCGACGCTCCTGCATACCAGCAGGGACAGAACCAGCTG |
| Codon | TATAACGAGCTGAATCTGGGCCGGAGAGAGGAATACGACGTGCTGGACAAAAGGCGGGGC |
| optimized | CGGGACCCCGAAATGGGAGGGAAGCCACGACGGAAAAACCCCCAGGAGGGCCTGTACAAT |
| CD3ζ signaling | GAGCTGCAAAAGGACAAAATGGCCGAGGCTTATTCTGAAATCGGGATGAAGGGAGAGAGA |
| domian | AGGCGCGGAAAAGGCCACGATGGCCTGTACCAGGGGCTGAGCACCGCTACAAAGGACACC |
| | TATGATGCACTGCACATGCAGGCCCTGCCCCCTCGG |
| SEQ ID NO: 16 | GDVEXNPGP |
| 2A cleavage | |
| motif | |
| SEQ ID NO: 17 | GSGEGRGSLLTCGDVEENPGP |
| T2A cleavage | |
| site | |
| SEQ ID NO: 18 | GGCTCAGGTGAGGGGCGCGGGAGCCTGCTGACTTGTGGGGATGTAGAGGAAAATCCTGGT |
| T2A cleavage | CCT |
| site | |
| SEQ ID NO: 19 | GSGATNFSLLKQAGDVEENPGP |
| P2A cleavage | |
| site | |
| SEQ ID NO: 20 | GSGQCTNYALLKLAGDVESNPGP |
| E2A cleavage | |
| site | |
| SEQ ID NO: 21 | GSGVKQTLNFDLLKLAGDVESNPGP |
| F2A cleavage | |
| site | |
| SEQ ID NO: 22 | MRISKPHLRSISIQCYLCLLLNSHFLTEAGIHVFILGCFSAGLPKTEANWVNVISDLKKI |
| IL-15 | EDLIQSMHIDATLYTESDVHPSCKVTAMKCFLLELQVISLESGDASIHDTVENLIILANN |
| | SLSSNGNVTESGCKECEELEEKNIKEFLQSFVHIVQMFINTS- |
| SEQ ID NO: 23 | ATGAGAATCAGCAAACCACACCTCCGGAGCATATCAATCCAGTGTTACTTGTGCCTTCTT |
| IL-15 | TTGAACTCCCATTTCCTCACCGAGGCAGGCATTCATGTGTTCATATTGGGGTGCTTTAGT |
| | GCTGGGCTTCCGAAAACGGAAGCTAACTGGGTAAACGTCATCAGTGACCTTAAAAAAATT |
| | GAGGATCTTATCCAATCAATGCACATCGACGCGACTCTCTACACAGAATCTGACGTACAC |
| | CCGTCATGCAAAGTCACGGCAATGAAGTGTTTTCTTCTCGAGCTCCAAGTAATTTCCCTG |
| | GAGTCTGGCGATGCCTCCATCCACGATACGGTTGAAAATCTGATTATATTGGCCAACAAT |
| | AGCCTCAGTTCTAACGGTAACGTGACTGAAAGTGGCTGCAAAGAGTGCGAAGAGCTCGAA |
| | GAAAAGAATATCAAGGAGTTCCTCCAATCATTTGTTCACATTGTGCAAATGTTTATCAAC |
| | ACCTCTTGA |
| SEQ ID NO: 24 | ATGCGCATAAGTAAGCCTCATCTGCGGTCCATTTCTATACAATGTTATCTGTGCTTGCTT |
| IL-15 | TTGAACTCCCACTTTCTTACGGAAGCAGGCATTCATGTGTTCATTCTGGGTTGTTTTTCt |
| | GCCGGGCTGCCCAAAACCGAGGCCAACTGGGTCAACGTGATCAGCGACCTCAAGAAGATC |
| | GAGGATTTGATTCAAAGTATGCATATAGACGCCACACTCTATACTGAGTCCGACGTTCAC |
| | CCGAGTTGTAAAGTTACGGCTATGAAGTGCTTTTTGTTGGAACTCCAGGTGATTTCCCTT |
| | GAATCCGGCGATGCGAGCATCCACGATACGGTAGAGAATCTTATTATTCTGGCGAATAAT |
| | TCTCTGTCTTCAAATGGGAATGTAACTGAGAGCGGTTGTAAAGAATGCGAAGAACTTGAA |
| | GAAAAGAATATCAAGGAATTTCTTCAGAGTTTCGTGCATATTGTTCAAATGTTCATCAAC |
| | ACATCCTGA |
| SEQ ID NO: 25 | RSKRSRLLHSDYMNMTPRRPGPTRKHYQPYAPPRDFAAYRSERVQPLEENVGNAARPRFE |
| CD28/OX40L/CDζ | RNKRVKFSRSADAPAYQQGQNQLYNELNLGRREEYDVLDKRRGRDPEMGGKPRRKNPQEG |
| | LYNELQKDKMAEAYSEIGMKGERRRGKGHDGLYQGLSTATKDTYDALHMQALPPR |
| SEQ ID NO: 26 | RSKRSRLLHSDYMNMTPRRPGPTRKHYQPYAPPRDFAAYRSERVQPLEENVGNAARPRFE |
| CD28/OX40L/ | RNKRVKFSRSADAPAYQQGQNQLYNELNLGRREEYDVLDKRRGRDPEMGGKPRRKNPQEG |
| CDζ/T2A/IL1-5 | LYNELQKDKMAEAYSEIGMKGERRRGKGHDGLYQGLSTATKDTYDALHMQALPPRGSGEG |
| | RGSLLTCGDVEENPGPMRISKPHLRSISIQCYLCLLLNSHFLTEAGIHVFILGCFSAGLP |
| | KTEANWVNVISDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHIVQMFINTS- |
| SEQ ID NO: 27 | MALPVTALLLPLALLLHAARP |
| CD8α signal | |
| sequence | |
| SEQ ID NO: 28 | ATGGCCTTACCAGTGACCGCCTTGCTCCTGCCGCTGGCCTTGCTGCTCCACGCCGCCAGG |
| CD8α signal | CCG |
| sequence | |
| SEQ ID NO: 29 | ATGGCACTTCCTGTTACAGCCCTCCTGCTCCCACTGGCTTTGCTGCTGCATGCTGCACGA |
| Codon | CCG |
| Optimized | |
| CD8α signal | |
| sequence | |
| SEQ ID NO: 30 | DIQMTQTTSSLSASLGDRVTISCRASQDISKYLNWYQQKPDGTVKLLIYHTSRLHSGVPS |
| anti-CD19 scFv | RFSGSGSGTDYSLTISNLEQEDIATYFCQQGNTLPYTFGGGTKLEITGGGGSGGGGSGGG |
| | GSEVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTSVTV |
| | SS |
| SEQ ID NO: 31 | GACATTCAAATGACACAAACTACGAGCTCCCTCTCCGCCTCCCTCGGTGACAGGGTGACC |
| anti-CD19 scFv | ATCTCATGTCGGGCTAGTCAGGACATAAGCAAATACTTGAACTGGTACCAGCAAAAGCCA |
| | GACGGGACCGTGAAGCTGCTCATCTATCACACTAGCCGCCTTCACTCTGGGGTCCCCTCT |
| | CGCTTTAGCGGTTCCGGCTCTGGTACAGACTATTCCCTTACTATTTCAAATCTTGAGCAG |
| | GAAGATATCGCTACATATTTTTGTCAACAGGGGAATACTCTTCCGTATACATTCGGGGGC |
| | GGAACAAAACTTGAAATTACAGGCGGCGGTGGTTCAGGAGGTGGTGGGAGCGGTGGGGGG |
| | GGATCAGAaGTtAAGCTGCAAGAATCAGGGCCCGGACTGGTAGCGCCCAGTCAAAGTCTT |
| | TCTGTTACTTGTACAGTATCCGGGGTGTCCCTTCCAGATTACGGGGTATCTTGGATTAGG |
| | CAACCTCCTCGGAAAGGATTGGAGTGGTTGGGTGTTATCTGGGGCAGTGAAACCACTTAC |
| | TATAATAGTGCTCTTAAAAGTAGACTCACCATAATAAAGGATAATAGCAAGAGCCAGGTA |
| | TTCCTGAAAATGAACAGCTTGCAAACGGATGATACCGCTATCTACTACTGCGCCAAACAC |
| | TATTATTATGGCGGTAGTTACGCTATGGATTATTGGGGCCAAGGaACGTCCGTCACCGTT |
| | AGTAGC |
| SEQ ID NO: 32 | EPKSCDKTHTCP |
| IgG1 hinge | |
| SEQ ID NO: 33 | GAGCCAAAGTCTTGCGACAAGACTCATACCTGCCCG |
| IgG1 hinge | |
| SEQ ID NO: 34 | PCPAPPVAGPSVFLFPPKPKDTLMISRTPEVTCVVVDVSHEDPEVKFNWYVDGVEVHNAK |
| Spacer (IgG1, | TKPREEQYQSTYRVVSVLTVLHQDWLNGKEYKCKVSNKALPAPIEKTISKAKGQPREPQV |
| CH2 (mu)-CH3 | YTLPPSRDELTKNQVSLTCLVKGFYPSDIAVEWESNGQPENNYKTTPPVLDSDGSFFLYS |
| | KLTVDKSRWQQGNVFSCSVMHEALHNHYTQKSLSLSPGK |
| SEQ ID NO: 35 | CCTTGTCCCGCACCACCTGTGGCAGGTCCCAGCGTGTTTCTGTTTCCGCCCAAGCCCAAG |
| Spacer (IgG1, | GACACACTCATGATCTCTAGGACACCCGAGGTTACATGCGTCGTGGTTGATGTTTCTCAT |
| CH2 (mu)-CH3 | GAAGACCCCGAAGTGAAGTTTAACTGGTATGTCGACGGAGTTGAGGTCCATAATGCTAAA |
| | ACAAAACCACGCGAGGAACAATATCAAAGCACCTATCGGGTCGTTAGTGTCCTTACTGTA |
| | CTTCACCAGGACTGGCTCAACGGAAAGGAATATAAGTGCAAAGTCAGCAATAAAGCCTTG |
| | CCAGCGCCCATCGAAAAAACTATCAGCAAGGCCAAG |
| SEQ ID NO: 36 | PCPAPELLGGPSVFLFPPKPKDTLMISRTPEVTCVVVDVSHEDPEVKFNWYVDGVEVHNA |
| Spacer (IgG1, | KTKPREEQYNSTYRVVSVLTVLHQDWLNGKEYKCKVSNKALPAPIEKTISKAKGQPREPQ |
| CH2 (mu)-CH3 | VYTLPPSRDELTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 37 | CCTTGTCCCGCACCACCTGTGGCAGGTCCCAGCGTGTTTCTGTTTCCGCCCAAGCCCAAG |
| Spacer (IgG1, | GACACACTCATGATCTCTAGGACACCCGAGGTTACATGCGTCGTGGTTGATGTTTCTCAT |
| CH2 (mu)-CH3 | GAAGACCCCGAAGTGAAGTTTAACTGGTATGTCGACGGAGTTGAGGTCCATAATGCTAAA |
| | ACAAAACCACGCGAGGAACAATATCAAAGCACCTATCGGGTCGTTAGTGTCCTTACTGTA |
| | CTTCACCAGGACTGGCTCAACGGAAAGGAATATAAGTGCAAAGTCAGCAATAAAGCCTTG |
| | CCAGCGCCCATCGAAAAAACTATCAGCAAGGCCAAGGGCCAACCCAGAGAACCCCAAGTG |
| | TACACACTCCCTCCTAGCCGGGATGAATTGACCAAAAATCAaGTCtctCTCACTTGTCTG |
| | GTAAAGGGATTCTATCCAAGTGATATTGCTGTCGAATGGGAGAGCAACGGGCAACCGGAG |
| | AACAATTACAAAACGACACCTCCCGTCCTTGATAGTGACGGGTCCTTCTTCCTGTATTCA |
| | AAGCTTACAGTCGACAAAAGTCGCTGGCAGCAGGGGAATGTCTTTAGCTGTAGTGTCATG |
| | CACGAAGCTTTGCATAACCACTACACGCAAAAGTCTCTTAGCCTGTCCCCAGGAAAG |
| SEQ ID NO: 38 | FWVLVVVGGVLACYSLLVTVAFIIFWV |
| CD28 | |
| transmembrane | |
| domain | |
| SEQ ID NO: 39 | TTCTGGGTACTGGTGGTTGTGGGCGGCGTTCTGGCTTGCTATTCACTCCTCGTTACGGTT |
| CD28 | GCATTTATAATATTTTGGGTA |
| transmembrane | |
| domain | |
| SEQ ID NO: 40 | IYIWAPLAGTCGVLLLSLVITLYC |
| CD8 trans- | |
| membrane | |
| domain | |
| SEQ ID NO: 41 | ATCTACATTTGGGCCCCTCTGGCTGGAACATGTGGCGTGCTGCTGCTGTCCCTGGTCATT |
| CD8 trans- | ACTCTGTATTGT |
| membrane | |
| domain | |
| SEQ ID NO: 42 | MALPVTALLLPLALLLHAARPDIQMTQTTSSLSASLGDRVTISCRASQDISKYLNWYQQK |
| CD19 CAR-WT-28 | PDGTVKLLIYHTSRLHSGVPSRFSGSGSGTDYSLTISNLEQEDIATYFCQQGNTLPYTFG |
| (with signal) | GGTKLEITGGGGSGGGGSGGGGSEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPELLGGPSVFLFPPKPKDTLM |
| | ISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEAL |
| | HNHYTQKSLSLSPGKFWVLVVVGGVLACYSLLVTVAFIIFWVRSKRSRLLHSDYMNMTPR |
| | RPGPTRKHYQPYAPPRDFAAYRSERVQPLEENVGNAARPRFERNKRVKFSRSADAPAYQQ |
| | GQNQLYNELNLGRREEYDVLDKRRGRDPEMGGKPRRKNPQEGLYNELQKDKMAEAYSEIG |
| | MKGERRRGKGHDGLYQGLSTATKDTYDALHMQALPPR |
| SEQ ID NO: 43 | ATGGCACTTCCTGTTACAGCCCTCCTGCTCCCACTGGCTTTGCTGCTGCATGCTGCACGA |
| CD19 CAR-WT-28 | CCGGACATTCAAATGACACAAACTACGAGCTCCCTCTCCGCCTCCCTCGGTGACAGGGTG |
| | ACCATCTCATGTCGGGCTAGTCAGGACATAAGCAAATACTTGAACTGGTACCAGCAAAAG |
| | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | GAGTTGCTGGGTGGTCCCAGCGTGTTTCTGTTTCCGCCCAAGCCCAAGGACACACTCATG |
| | ATCTCTAGGACACCCGAGGTTACATGCGTCGTGGTTGATGTTTCTCATGAAGACCCCGAA |
| | GTGAAGTTTAACTGGTATGTCGACGGAGTTGAGGTCCATAATGCTAAAACAAAACCACGC |
| | GAGGAACAATATAATAGCACCTATCGGGTCGTTAGTGTCCTTACTGTACTTCACCAGGAC |
| | TGGCTCAACGGAAAGGAATATAAGTGCAAAGTCAGCAATAAAGCCTTGCCAGCGCCCATC |
| | GAAAAAACTATCAGCAAGGCCAAGGGCCAACCCAGAGAACCCCAAGTGTACACACTCCCT |
| | CCTAGCCGGGATGAATTGACCAAAAATCAaGTCtctCTCACTTGTCTGGTAAAGGGATTC |
| | TATCCAAGTGATATTGCTGTCGAATGGGAGAGCAACGGGCAACCGGAGAACAATTACAAA |
| | ACGACACCTCCCGTCCTTGATAGTGACGGGTCCTTCTTCCTGTATTCAAAGCTTACAGTC |
| | GACAAAAGTCGCTGGCAGCAGGGGAATGTCTTTAGCTGTAGTGTCATGCACGAAGCTTTG |
| | CATAACCACTACACGCAAAAGTCTCTTAGCCTGTCCCCAGGAAAGTTCTGGGTACTGGTG |
| | GTTGTGGGCGGCGTTCTGGCTTGCTATTCACTCCTCGTTACGGTTGCATTTATAATATTT |
| | TGGGTACGCTCAAAGCGCTCAAGATTGCTCCATTCTGATTACATGAACATGACCCCTCGC |
| | CGGCCGGGTCCCACTCGCAAACACTACCAACCTTATGCCCCTCCGCGGGATTTCGCGGCC |
| | TACAGGTCAGAGAGAGTACAACCTCTTGAGGAGAATGTCGGCAACGCTGCCAGGCCGCGG |
| | TTTGAGCGAAATAAGCGAGTCAAGTTTTCTCGAAGTGCCGACGCTCCTGCCTATCAGCAA |
| | GGCCAGAATCAACTGTATAACGAGCTTAACTTGGGCCGGAGGGAGGAGTACGATGTGCTG |
| | GATAAGCGCAGGGGACGGGACCCGGAAATGGGCGGAAAGCCCAGGCGGAAAAACCCACAG |
| | GAAGGACTCTACAATGAACTCCAAAAGGACAAAATGGCAGAGGCCTATTCAGAGATTGGG |
| | ATGAAGGGAGAAAGGAGGCGAGGAAAAGGGCACGACGGCTTGTATCAGGGCTTGTCTACG |
| | GCCACTAAAGACACCTATGATGCCCTGCATATGCAGGCACTTCCCCCCAGA |
| SEQ ID NO: 44 | MALPVTALLLPLALLLHAARPDIQMTQTTSSLSASLGDRVTISCRASQDISKYLNWYQQK |
| CD19 CAR-WT-28 | PDGTVKLLIYHTSRLHSGVPSRFSGSGSGTDYSLTISNLEQEDIATYFCQQGNTLPYTFG |
| with T2A and | GGTKLEITGGGGSGGGGSGGGGSEVKLQESGPGLVAPSQSLSVTCTVSGVSLPDYGVSWI |
| IL-15 | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPELLGGPSVFLFPPKPKDTLM |
| | ISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEAL |
| | HNHYTQKSLSLSPGKFWVLVVVGGVLACYSLLVTVAFIIFWVRSKRSRLLHSDYMNMTPR |
| | RPGPTRKHYQPYAPPRDFAAYRSERVQPLEENVGNAARPRFERNKRVKFSRSADAPAYQQ |
| | GQNQLYNELNLGRREEYDVLDKRRGRDPEMGGKPRRKNPQEGLYNELQKDKMAEAYSEIG |
| | MKGERRRGKGHDGLYQGLSTATKDTYDALHMQALPPRGSGEGRGSLLTCGDVEENPGPMR |
| | ISKPHLRSISIQCYLCLLLNSHFLTEAGIHVFILGCFSAGLPKTEANWVNVISDLKKIED |
| | LIQSMHIDATLYTESDVHPSCKVTAMKCFLLELQVISLESGDASIHDTVENLIILANNSL |
| | SSNGNVTESGCKECEELEEKNIKEFLQSFVHIVQMFINTS- |
| SEQ ID NO: 45 | ATGGCACTTCCTGTTACAGCCCTCCTGCTCCCACTGGCTTTGCTGCTGCATGCTGCACGA |
| CD19 CAR-WT-28 | CCGGACATTCAAATGACACAAACTACGAGCTCCCTCTCCGCCTCCCTCGGTGACAGGGTG |
| with T2A and | ACCATCTCATGTCGGGCTAGTCAGGACATAAGCAAATACTTGAACTGGTACCAGCAAAAG |
| IL-15 | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | GAGTTGCTGGGTGGTCCCAGCGTGTTTCTGTTTCCGCCCAAGCCCAAGGACACACTCATG |
| | ATCTCTAGGACACCCGAGGTTACATGCGTCGTGGTTGATGTTTCTCATGAAGACCCCGAA |
| | GTGAAGTTTAACTGGTATGTCGACGGAGTTGAGGTCCATAATGCTAAAACAAAACCACGC |
| | GAGGAACAATATAATAGCACCTATCGGGTCGTTAGTGTCCTTACTGTACTTCACCAGGAC |
| | TGGCTCAACGGAAAGGAATATAAGTGCAAAGTCAGCAATAAAGCCTTGCCAGCGCCCATC |
| | GAAAAAACTATCAGCAAGGCCAAGGGCCAACCCAGAGAACCCCAAGTGTACACACTCCCT |
| | CCTAGCCGGGATGAATTGACCAAAAATCAaGTCtctCTCACTTGTCTGGTAAAGGGATTC |
| | TATCCAAGTGATATTGCTGTCGAATGGGAGAGCAACGGGCAACCGGAGAACAATTACAAA |
| | ACGACACCTCCCGTCCTTGATAGTGACGGGTCCTTCTTCCTGTATTCAAAGCTTACAGTC |
| | GACAAAAGTCGCTGGCAGCAGGGGAATGTCTTTAGCTGTAGTGTCATGCACGAAGCTTTG |
| | CATAACCACTACACGCAAAAGTCTCTTAGCCTGTCCCCAGGAAAGTTCTGGGTACTGGTG |
| | GTTGTGGGCGGCGTTCTGGCTTGCTATTCACTCCTCGTTACGGTTGCATTTATAATATTT |
| | TGGGTACGCTCAAAGCGCTCAAGATTGCTCCATTCTGATTACATGAACATGACCCCTCGC |
| | CGGCCGGGTCCCACTCGCAAACACTACCAACCTTATGCCCCTCCGCGGGATTTCGCGGCC |
| | TACAGGTCAGAGAGAGTACAACCTCTTGAGGAGAATGTCGGCAACGCTGCCAGGCCGCGG |
| | TTTGAGCGAAATAAGCGAGTCAAGTTTTCTCGAAGTGCCGACGCTCCTGCCTATCAGCAA |
| | GGCCAGAATCAACTGTATAACGAGCTTAACTTGGGCCGGAGGGAGGAGTACGATGTGCTG |
| | GATAAGCGCAGGGGACGGGACCCGGAAATGGGCGGAAAGCCCAGGCGGAAAAACCCACAG |
| | GAAGGACTCTACAATGAACTCCAAAAGGACAAAATGGCAGAGGCCTATTCAGAGATTGGG |
| | ATGAAGGGAGAAAGGAGGCGAGGAAAAGGGCACGACGGCTTGTATCAGGGCTTGTCTACG |
| | GCCACTAAAGACACCTATGATGCCCTGCATATGCAGGCACTTCCCCCCAGAGGAAGTGGG |
| | GAAGGCCGAGGTTCATTGCTCACCTGTGGCGATGTGGAAGAAAATCCAGGTCCGATGCGC |
| | ATAAGTAAGCCTCATCTGCGGTCCATTTCTATACAATGTTATCTGTGCTTGCTTTTGAAC |
| | TCCCACTTTCTTACGGAAGCAGGCATTCATGTGTTCATTCTGGGTTGTTTTTCtGCCGGG |
| | CTGCCCAAAACCGAGGCCAACTGGGTCAACGTGATCAGCGACCTCAAGAAGATCGAGGAT |
| | TTGATTCAAAGTATGCATATAGACGCCACACTCTATACTGAGTCCGACGTTCACCCGAGT |
| | TGTAAAGTTACGGCTATGAAGTGCTTTTTGTTGGAACTCCAGGTGATTTCCCTTGAATCC |
| | GGCGATGCGAGCATCCACGATACGGTAGAGAATCTTATTATTCTGGCGAATAATTCTCTG |
| | TCTTCAAATGGGAATGTAACTGAGAGCGGTTGTAAAGAATGCGAAGAACTTGAAGAAAAG |
| | AATATCAAGGAATTTCTTCAGAGTTTCGTGCATATTGTTCAAATGTTCATCAACACATCC |
| | TGA |
| SEQ ID NO: 46 | MALPVTALLLPLALLLHAARPDIQMTQTTSSLSASLGDRVTISCRASQDISKYLNWYQQK |
| CD19 CAR-MU-28 | PDGTVKLLIYHTSRLHSGVPSRESGSGSGTDYSLTISNLEQEDIATYFCQQGNTLPYTFG |
| (with signal) | GGTKLEITGGGGSGGGGSGGGGSEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPPVAGPSVFLFPPKPKDTLMI |
| | SRTPEVTCVVVDVSHEDPEVKFNWYVDGVEVHNAKTKPREEQYQSTYRVVSVLTVLHQDW |
| | LNGKEYKCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSVMHEALH |
| | NHYTQKSLSLSPGKFWVLVVVGGVLACYSLLVTVAFIIFWVRSKRSRLLHSDYMNMTPRR |
| | PGPTRKHYQPYAPPRDFAAYRSERVQPLEENVGNAARPRFERNKRVKFSRSADAPAYQQG |
| | QNQLYNELNLGRREEYDVLDKRRGRDPEMGGKPRRKNPQEGLYNELQKDKMAEAYSEIGM |
| | KGERRRGKGHDGLYQGLSTATKDTYDALHMQALPPR |
| SEQ ID NO: 47 | ATGGCACTTCCTGTTACAGCCCTCCTGCTCCCACTGGCTTTGCTGCTGCATGCTGCACGA |
| CD19 CAR-MU-28 | CCGGACATTCAAATGACACAAACTACGAGCTCCCTCTCCGCCTCCCTCGGTGACAGGGTG |
| | ACCATCTCATGTCGGGCTAGTCAGGACATAAGCAAATACTTGAACTGGTACCAGCAAAAG |
| | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | CCTGTGGCAGGTCCCAGCGTGTTTCTGTTTCCGCCCAAGCCCAAGGACACACTCATGATC |
| | TCTAGGACACCCGAGGTTACATGCGTCGTGGTTGATGTTTCTCATGAAGACCCCGAAGTG |
| | AAGTTTAACTGGTATGTCGACGGAGTTGAGGTCCATAATGCTAAAACAAAACCACGCGAG |
| | GAACAATATCAAAGCACCTATCGGGTCGTTAGTGTCCTTACTGTACTTCACCAGGACTGG |
| | CTCAACGGAAAGGAATATAAGTGCAAAGTCAGCAATAAAGCCTTGCCAGCGCCCATCGAA |
| | AAAACTATCAGCAAGGCCAAGGGCCAACCCAGAGAACCCCAAGTGTACACACTCCCTCCT |
| | AGCCGGGATGAATTGACCAAAAATCAaGTCtctCTCACTTGTCTGGTAAAGGGATTCTAT |
| | CCAAGTGATATTGCTGTCGAATGGGAGAGCAACGGGCAACCGGAGAACAATTACAAAACG |
| | ACACCTCCCGTCCTTGATAGTGACGGGTCCTTCTTCCTGTATTCAAAGCTTACAGTCGAC |
| | AAAAGTCGCTGGCAGCAGGGGAATGTCTTTAGCTGTAGTGTCATGCACGAAGCTTTGCAT |
| | AACCACTACACGCAAAAGTCTCTTAGCCTGTCCCCAGGAAAGTTCTGGGTACTGGTGGTT |
| | GTGGGCGGCGTTCTGGCTTGCTATTCACTCCTCGTTACGGTTGCATTTATAATATTTTGG |
| | GTACGCTCAAAGCGCTCAAGATTGCTCCATTCTGATTACATGAACATGACCCCTCGCCGG |
| | CCGGGTCCCACTCGCAAACACTACCAACCTTATGCCCCTCCGCGGGATTTCGCGGCCTAC |
| | AGGTCAGAGAGAGTACAACCTCTTGAGGAGAATGTCGGCAACGCTGCCAGGCCGCGGTTT |
| | GAGCGAAATAAGCGAGTCAAGTTTTCTCGAAGTGCCGACGCTCCTGCCTATCAGCAAGGC |
| | CAGAATCAACTGTATAACGAGCTTAACTTGGGCCGGAGGGAGGAGTACGATGTGCTGGAT |
| | AAGCGCAGGGGACGGGACCCGGAAATGGGCGGAAAGCCCAGGCGGAAAAACCCACAGGAA |
| | GGACTCTACAATGAACTCCAAAAGGACAAAATGGCAGAGGCCTATTCAGAGATTGGGATG |
| | AAGGGAGAAAGGAGGCGAGGAAAAGGGCACGACGGCTTGTATCAGGGCTTGTCTACGGCC |
| | ACTAAAGACACCTATGATGCCCTGCATATGCAGGCACTTCCCCCCAGA |
| SEQ ID NO: 48 | MALPVTALLLPLALLLHAARPDIQMTQTTSSLSASLGDRVTISCRASQDISKYLNWYQQK |
| CD19 CAR-MU-28 | PDGTVKLLIYHTSRLHSGVPSRFSGSGSGTDYSLTISNLEQEDIATYFCQQGNTLPYTFG |
| with T2A and | GGTKLEITGGGGSGGGGSGGGGSEVKLQESGPGLVAPSQSLSVTCTVSGVSLPDYGVSWI |
| IL-15 | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPPVAGPSVFLFPPKPKDTLMI |
| | SRTPEVTCVVVDVSHEDPEVKFNWYVDGVEVHNAKTKPREEQYQSTYRVVSVLTVLHQDW |
| | LNGKEYKCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSVMHEALH |
| | NHYTQKSLSLSPGKFWVLVVVGGVLACYSLLVTVAFIIFWVRSKRSRLLHSDYMNMTPRR |
| | PGPTRKHYQPYAPPRDFAAYRSERVQPLEENVGNAARPRFERNKRVKFSRSADAPAYQQG |
| | QNQLYNELNLGRREEYDVLDKRRGRDPEMGGKPRRKNPQEGLYNELQKDKMAEAYSEIGM |
| | KGERRRGKGHDGLYQGLSTATKDTYDALHMQALPPRGSGEGRGSLLTCGDVEENPGPMRI |
| | SKPHLRSISIQCYLCLLLNSHFLTEAGIHVFILGCFSAGLPKTEANWVNVISDLKKIEDL |
| | IQSMHIDATLYTESDVHPSCKVTAMKCFLLELQVISLESGDASIHDTVENLIILANNSLS |
| | SNGNVTESGCKECEELEEKNIKEFLQSFVHIVQMFINTS- |
| SEQ ID NO: 49 | ATGGCACTTCCTGTTACAGCCCTCCTGCTCCCACTGGCTTTGCTGCTGCATGCTGCACGA |
| CD19 CAR-MU-28 | CCGGACATTCAAATGACACAAACTACGAGCTCCCTCTCCGCCTCCCTCGGTGACAGGGTG |
| with T2A and | ACCATCTCATGTCGGGCTAGTCAGGACATAAGCAAATACTTGAACTGGTACCAGCAAAAG |
| IL-15 | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | CCTGTGGCAGGTCCCAGCGTGTTTCTGTTTCCGCCCAAGCCCAAGGACACACTCATGATC |
| | TCTAGGACACCCGAGGTTACATGCGTCGTGGTTGATGTTTCTCATGAAGACCCCGAAGTG |
| | AAGTTTAACTGGTATGTCGACGGAGTTGAGGTCCATAATGCTAAAACAAAACCACGCGAG |
| | GAACAATATCAAAGCACCTATCGGGTCGTTAGTGTCCTTACTGTACTTCACCAGGACTGG |
| | CTCAACGGAAAGGAATATAAGTGCAAAGTCAGCAATAAAGCCTTGCCAGCGCCCATCGAA |
| | AAAACTATCAGCAAGGCCAAGGGCCAACCCAGAGAACCCCAAGTGTACACACTCCCTCCT |
| | AGCCGGGATGAATTGACCAAAAATCAaGTCtctCTCACTTGTCTGGTAAAGGGATTCTAT |
| | CCAAGTGATATTGCTGTCGAATGGGAGAGCAACGGGCAACCGGAGAACAATTACAAAACG |
| | ACACCTCCCGTCCTTGATAGTGACGGGTCCTTCTTCCTGTATTCAAAGCTTACAGTCGAC |
| | AAAAGTCGCTGGCAGCAGGGGAATGTCTTTAGCTGTAGTGTCATGCACGAAGCTTTGCAT |
| | AACCACTACACGCAAAAGTCTCTTAGCCTGTCCCCAGGAAAGTTCTGGGTACTGGTGGTT |
| | GTGGGCGGCGTTCTGGCTTGCTATTCACTCCTCGTTACGGTTGCATTTATAATATTTTGG |
| | GTACGCTCAAAGCGCTCAAGATTGCTCCATTCTGATTACATGAACATGACCCCTCGCCGG |
| | CCGGGTCCCACTCGCAAACACTACCAACCTTATGCCCCTCCGCGGGATTTCGCGGCCTAC |
| | AGGTCAGAGAGAGTACAACCTCTTGAGGAGAATGTCGGCAACGCTGCCAGGCCGCGGTTT |
| | GAGCGAAATAAGCGAGTCAAGTTTTCTCGAAGTGCCGACGCTCCTGCCTATCAGCAAGGC |
| | CAGAATCAACTGTATAACGAGCTTAACTTGGGCCGGAGGGAGGAGTACGATGTGCTGGAT |
| | AAGCGCAGGGGACGGGACCCGGAAATGGGCGGAAAGCCCAGGCGGAAAAACCCACAGGAA |
| | GGACTCTACAATGAACTCCAAAAGGACAAAATGGCAGAGGCCTATTCAGAGATTGGGATG |
| | AAGGGAGAAAGGAGGCGAGGAAAAGGGCACGACGGCTTGTATCAGGGCTTGTCTACGGCC |
| | ACTAAAGACACCTATGATGCCCTGCATATGCAGGCACTTCCCCCCAGAGGAAGTGGGGAA |
| | GGCCGAGGTTCATTGCTCACCTGTGGCGATGTGGAAGAAAATCCAGGTCCGATGCGCATA |
| | AGTAAGCCTCATCTGCGGTCCATTTCTATACAATGTTATCTGTGCTTGCTTTTGAACTCC |
| | CACTTTCTTACGGAAGCAGGCATTCATGTGTTCATTCTGGGTTGTTTTTCtGCCGGGCTG |
| | CCCAAAACCGAGGCCAACTGGGTCAACGTGATCAGCGACCTCAAGAAGATCGAGGATTTG |
| | ATTCAAAGTATGCATATAGACGCCACACTCTATACTGAGTCCGACGTTCACCCGAGTTGT |
| | AAAGTTACGGCTATGAAGTGCTTTTTGTTGGAACTCCAGGTGATTTCCCTTGAATCCGGC |
| | GATGCGAGCATCCACGATACGGTAGAGAATCTTATTATTCTGGCGAATAATTCTCTGTCT |
| | TCAAATGGGAATGTAACTGAGAGCGGTTGTAAAGAATGCGAAGAACTTGAAGAAAAGAAT |
| | ATCAAGGAATTTCTTCAGAGTTTCGTGCATATTGTTCAAATGTTCATCAACACATCCTGA |
| SEQ ID NO: 50 | MALPVTALLLPLALLLHAARPDIQMTQTTSSLSASLGDRVTISCRASQDISKYLNWYQQK |
| CD19 CAR-WT-8 | PDGTVKLLIYHTSRLHSGVPSRFSGSGSGTDYSLTISNLEQEDIATYFCQQGNTLPYTFG |
| (with signal) | GGTKLEITGGGGSGGGGSGGGGSEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPELLGGPSVFLFPPKPKDTLM |
| | ISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEAL |
| | HNHYTQKSLSLSPGKIYIWAPLAGTCGVLLLSLVITLYCRSKRSRLLHSDYMNMTPRRPG |
| | PTRKHYQPYAPPRDFAAYRSERVQPLEENVGNAARPRFERNKRVKFSRSADAPAYQQGQN |
| | QLYNELNLGRREEYDVLDKRRGRDPEMGGKPRRKNPQEGLYNELQKDKMAEAYSEIGMKG |
| | ERRRGKGHDGLYQGLSTATKDTYDALHMQALPPR |
| SEQ ID NO: 51 | ATGGCACTTCCTGTTACAGCCCTCCTGCTCCCACTGGCTTTGCTGCTGCATGCTGCACGA |
| CD19 CAR-WT-8 | CCGGACATTCAAATGACACAAACTACGAGCTCCCTCTCCGCCTCCCTCGGTGACAGGGTG |
| | ACCATCTCATGTCGGGCTAGTCAGGACATAAGCAAATACTTGAACTGGTACCAGCAAAAG |
| | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | GAGTTGCTGGGTGGTCCCAGCGTGTTTCTGTTTCCGCCCAAGCCCAAGGACACACTCATG |
| | ATCTCTAGGACACCCGAGGTTACATGCGTCGTGGTTGATGTTTCTCATGAAGACCCCGAA |
| | GTGAAGTTTAACTGGTATGTCGACGGAGTTGAGGTCCATAATGCTAAAACAAAACCACGC |
| | GAGGAACAATATAATAGCACCTATCGGGTCGTTAGTGTCCTTACTGTACTTCACCAGGAC |
| | TGGCTCAACGGAAAGGAATATAAGTGCAAAGTCAGCAATAAAGCCTTGCCAGCGCCCATC |
| | GAAAAAACTATCAGCAAGGCCAAGGGCCAACCCAGAGAACCCCAAGTGTACACACTCCCT |
| | CCTAGCCGGGATGAATTGACCAAAAATCAaGTCtctCTCACTTGTCTGGTAAAGGGATTC |
| | TATCCAAGTGATATTGCTGTCGAATGGGAGAGCAACGGGCAACCGGAGAACAATTACAAA |
| | ACGACACCTCCCGTCCTTGATAGTGACGGGTCCTTCTTCCTGTATTCAAAGCTTACAGTC |
| | GACAAAAGTCGCTGGCAGCAGGGGAATGTCTTTAGCTGTAGTGTCATGCACGAAGCTTTG |
| | CATAACCACTACACGCAAAAGTCTCTTAGCCTGTCCCCAGGAAAGATCTACATTTGGGCC |
| | CCTCTGGCTGGAACATGTGGCGTGCTGCTGCTGTCCCTGGTCATTACTCTGTATTGTCGC |
| | TCAAAGCGCTCAAGATTGCTCCATTCTGATTACATGAACATGACCCCTCGCCGGCCGGGT |
| | CCCACTCGCAAACACTACCAACCTTATGCCCCTCCGCGGGATTTCGCGGCCTACAGGTCA |
| | GAGAGAGTACAACCTCTTGAGGAGAATGTCGGCAACGCTGCCAGGCCGCGGTTTGAGCGA |
| | AATAAGCGAGTCAAGTTTTCTCGAAGTGCCGACGCTCCTGCCTATCAGCAAGGCCAGAAT |
| | CAACTGTATAACGAGCTTAACTTGGGCCGGAGGGAGGAGTACGATGTGCTGGATAAGCGC |
| | AGGGGACGGGACCCGGAAATGGGCGGAAAGCCCAGGCGGAAAAACCCACAGGAAGGACTC |
| | TACAATGAACTCCAAAAGGACAAAATGGCAGAGGCCTATTCAGAGATTGGGATGAAGGGA |
| | GAAAGGAGGCGAGGAAAAGGGCACGACGGCTTGTATCAGGGCTTGTCTACGGCCACTAAA |
| | GACACCTATGATGCCCTGCATATGCAGGCACTTCCCCCCAGA |
| SEQ ID NO: 52 | MALPVTALLLPLALLLHAARPDIQMTQTTSSLSASLGDRVTISCRASQDISKYLNWYQQK |
| CD19 CAR-WT-8 | PDGTVKLLIYHTSRLHSGVPSRFSGSGSGTDYSLTISNLEQEDIATYFCQQGNTLPYTFG |
| with T2A and | GGTKLEITGGGGSGGGGSGGGGSEVKLQESGPGLVAPSQSLSVTCTVSGVSLPDYGVSWI |
| IL-15 | RQPPRKGLEWLGVIWGSETTYYNSALKSRLTIIKDNSKSQVFLKMNSLQTDDTAIYYCAK |
| (with signal) | HYYYGGSYAMDYWGQGTSVTVSSEPKSCDKTHTCPPCPAPELLGGPSVFLFPPKPKDTLM |
| | ISRTPEVTCVVVDVSHEDPEVKFNWYVDGVEVHNAKTKPREEQYNSTYRVVSVLTVLHQD |
| | WLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEAL |
| | HNHYTQKSLSLSPGKIYIWAPLAGTCGVLLLSLVITLYCRSKRSRLLHSDYMNMTPRRPG |
| | PTRKHYQPYAPPRDFAAYRSERVQPLEENVGNAARPRFERNKRVKFSRSADAPAYQQGQN |
| | QLYNELNLGRREEYDVLDKRRGRDPEMGGKPRRKNPQEGLYNELQKDKMAEAYSEIGMKG |
| | ERRRGKGHDGLYQGLSTATKDTYDALHMQALPPRGSGEGRGSLLTCGDVEENPGPMRISK |
| | PHLRSISIQCYLCLLLNSHFLTEAGIHVFILGCFSAGLPKTEANWVNVISDLKKIEDLIQ |
| | SMHIDATLYTESDVHPSCKVTAMKCFLLELQVISLESGDASIHDTVENLIILANNSLSSN |
| | GNVTESGCKECEELEEKNIKEFLQSFVHIVQMFINTS- |
| SEQ ID NO: 53 | ATGGCACTTCCTGTTACAGCCCTCCTGCTCCCACTGGCTTTGCTGCTGCATGCTGCACGA |
| CD19 CAR-WT-8 | CCGGACATTCAAATGACACAAACTACGAGCTCCCTCTCCGCCTCCCTCGGTGACAGGGTG |
| with T2A and | ACCATCTCATGTCGGGCTAGTCAGGACATAAGCAAATACTTGAACTGGTACCAGCAAAAG |
| IL-15 | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | GAGTTGCTGGGTGGTCCCAGCGTGTTTCTGTTTCCGCCCAAGCCCAAGGACACACTCATG |
| | ATCTCTAGGACACCCGAGGTTACATGCGTCGTGGTTGATGTTTCTCATGAAGACCCCGAA |
| | GTGAAGTTTAACTGGTATGTCGACGGAGTTGAGGTCCATAATGCTAAAACAAAACCACGC |
| | GAGGAACAATATAATAGCACCTATCGGGTCGTTAGTGTCCTTACTGTACTTCACCAGGAC |
| | TGGCTCAACGGAAAGGAATATAAGTGCAAAGTCAGCAATAAAGCCTTGCCAGCGCCCATC |
| | GAAAAAACTATCAGCAAGGCCAAGGGCCAACCCAGAGAACCCCAAGTGTACACACTCCCT |
| | CCTAGCCGGGATGAATTGACCAAAAATCAaGTCtctCTCACTTGTCTGGTAAAGGGATTC |
| | TATCCAAGTGATATTGCTGTCGAATGGGAGAGCAACGGGCAACCGGAGAACAATTACAAA |
| | ACGACACCTCCCGTCCTTGATAGTGACGGGTCCTTCTTCCTGTATTCAAAGCTTACAGTC |
| | GACAAAAGTCGCTGGCAGCAGGGGAATGTCTTTAGCTGTAGTGTCATGCACGAAGCTTTG |
| | CATAACCACTACACGCAAAAGTCTCTTAGCCTGTCCCCAGGAAAGATCTACATTTGGGCC |
| | CCTCTGGCTGGAACATGTGGCGTGCTGCTGCTGTCCCTGGTCATTACTCTGTATTGTCGC |
| | TCAAAGCGCTCAAGATTGCTCCATTCTGATTACATGAACATGACCCCTCGCCGGCCGGGT |
| | CCCACTCGCAAACACTACCAACCTTATGCCCCTCCGCGGGATTTCGCGGCCTACAGGTCA |
| | GAGAGAGTACAACCTCTTGAGGAGAATGTCGGCAACGCTGCCAGGCCGCGGTTTGAGCGA |
| | AATAAGCGAGTCAAGTTTTCTCGAAGTGCCGACGCTCCTGCCTATCAGCAAGGCCAGAAT |
| | CAACTGTATAACGAGCTTAACTTGGGCCGGAGGGAGGAGTACGATGTGCTGGATAAGCGC |
| | AGGGGACGGGACCCGGAAATGGGCGGAAAGCCCAGGCGGAAAAACCCACAGGAAGGACTC |
| | TACAATGAACTCCAAAAGGACAAAATGGCAGAGGCCTATTCAGAGATTGGGATGAAGGGA |
| | GAAAGGAGGCGAGGAAAAGGGCACGACGGCTTGTATCAGGGCTTGTCTACGGCCACTAAA |
| | GACACCTATGATGCCCTGCATATGCAGGCACTTCCCCCCAGAGGAAGTGGGGAAGGCCGA |
| | GGTTCATTGCTCACCTGTGGCGATGTGGAAGAAAATCCAGGTCCGATGCGCATAAGTAAG |
| | CCTCATCTGCGGTCCATTTCTATACAATGTTATCTGTGCTTGCTTTTGAACTCCCACTTT |
| | CTTACGGAAGCAGGCATTCATGTGTTCATTCTGGGTTGTTTTTCtGCCGGGCTGCCCAAA |
| | ACCGAGGCCAACTGGGTCAACGTGATCAGCGACCTCAAGAAGATCGAGGATTTGATTCAA |
| | AGTATGCATATAGACGCCACACTCTATACTGAGTCCGACGTTCACCCGAGTTGTAAAGTT |
| | ACGGCTATGAAGTGCTTTTTGTTGGAACTCCAGGTGATTTCCCTTGAATCCGGCGATGCG |
| | AGCATCCACGATACGGTAGAGAATCTTATTATTCTGGCGAATAATTCTCTGTCTTCAAAT |
| | GGGAATGTAACTGAGAGCGGTTGTAAAGAATGCGAAGAACTTGAAGAAAAGAATATCAAG |
| | GAATTTCTTCAGAGTTTCGTGCATATTGTTCAAATGTTCATCAACACATCCTGA |
| SEQ ID NO: 54 | MALPVTALLLPLALLLHAARPDIQMTQTTSSLSASLGDRVTISCRASQDISKYLNWYQQK |
| CD19 CAR-MU-8 | PDGTVKLLIYHTSRLHSGVPSRFSGSGSGTDYSLTISNLEQEDIATYFCQQGNTLPYTFG |
| (with signal) | GGTKLEITGGGGSGGGGSGGGGSEVKLQESGPGLVAPSQSLSVTCTVSGVSLPDYGVSWI |
| | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPPVAGPSVFLFPPKPKDTLMI |
| | SRTPEVTCVVVDVSHEDPEVKFNWYVDGVEVHNAKTKPREEQYQSTYRVVSVLTVLHQDW |
| | LNGKEYKCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSVMHEALH |
| | NHYTQKSLSLSPGKIYIWAPLAGTCGVLLLSLVITLYCRSKRSRLLHSDYMNMTPRRPGP |
| | TRKHYQPYAPPRDFAAYRSERVQPLEENVGNAARPRFERNKRVKFSRSADAPAYQQGQNQ |
| | LYNELNLGRREEYDVLDKRRGRDPEMGGKPRRKNPQEGLYNELQKDKMAEAYSEIGMKGE |
| | RRRGKGHDGLYQGLSTATKDTYDALHMQALPPR |
| SEQ ID NO: 55 | ATGGCACTTCCTGTTACAGCCCTCCTGCTCCCACTGGCTTTGCTGCTGCATGCTGCACGA |
| CD19 CAR-MU-8 | CCGGACATTCAAATGACACAAACTACGAGCTCCCTCTCCGCCTCCCTCGGTGACAGGGTG |
| | ACCATCTCATGTCGGGCTAGTCAGGACATAAGCAAATACTTGAACTGGTACCAGCAAAAG |
| | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | CCTGTGGCAGGTCCCAGCGTGTTTCTGTTTCCGCCCAAGCCCAAGGACACACTCATGATC |
| | TCTAGGACACCCGAGGTTACATGCGTCGTGGTTGATGTTTCTCATGAAGACCCCGAAGTG |
| | AAGTTTAACTGGTATGTCGACGGAGTTGAGGTCCATAATGCTAAAACAAAACCACGCGAG |
| | GAACAATATCAAAGCACCTATCGGGTCGTTAGTGTCCTTACTGTACTTCACCAGGACTGG |
| | CTCAACGGAAAGGAATATAAGTGCAAAGTCAGCAATAAAGCCTTGCCAGCGCCCATCGAA |
| | AAAACTATCAGCAAGGCCAAGGGCCAACCCAGAGAACCCCAAGTGTACACACTCCCTCCT |
| | AGCCGGGATGAATTGACCAAAAATCAaGTCtctCTCACTTGTCTGGTAAAGGGATTCTAT |
| | CCAAGTGATATTGCTGTCGAATGGGAGAGCAACGGGCAACCGGAGAACAATTACAAAACG |
| | ACACCTCCCGTCCTTGATAGTGACGGGTCCTTCTTCCTGTATTCAAAGCTTACAGTCGAC |
| | AAAAGTCGCTGGCAGCAGGGGAATGTCTTTAGCTGTAGTGTCATGCACGAAGCTTTGCAT |
| | AACCACTACACGCAAAAGTCTCTTAGCCTGTCCCCAGGAAAGATCTACATTTGGGCCCCT |
| | CTGGCTGGAACATGTGGCGTGCTGCTGCTGTCCCTGGTCATTACTCTGTATTGTCGCTCA |
| | AAGCGCTCAAGATTGCTCCATTCTGATTACATGAACATGACCCCTCGCCGGCCGGGTCCC |
| | ACTCGCAAACACTACCAACCTTATGCCCCTCCGCGGGATTTCGCGGCCTACAGGTCAGAG |
| | AGAGTACAACCTCTTGAGGAGAATGTCGGCAACGCTGCCAGGCCGCGGTTTGAGCGAAAT |
| | AAGCGAGTCAAGTTTTCTCGAAGTGCCGACGCTCCTGCCTATCAGCAAGGCCAGAATCAA |
| | CTGTATAACGAGCTTAACTTGGGCCGGAGGGAGGAGTACGATGTGCTGGATAAGCGCAGG |
| | GGACGGGACCCGGAAATGGGCGGAAAGCCCAGGCGGAAAAACCCACAGGAAGGACTCTAC |
| | AATGAACTCCAAAAGGACAAAATGGCAGAGGCCTATTCAGAGATTGGGATGAAGGGAGAA |
| | AGGAGGCGAGGAAAAGGGCACGACGGCTTGTATCAGGGCTTGTCTACGGCCACTAAAGAC |
| | ACCTATGATGCCCTGCATATGCAGGCACTTCCCCCCAGA |
| SEQ ID NO: 56 | MALPVTALLLPLALLLHAARPDIQMTQTTSSLSASLGDRVTISCRASQDISKYLNWYQQK |
| CD19 CAR-MU-8 | PDGTVKLLIYHTSRLHSGVPSRFSGSGSGTDYSLTISNLEQEDIATYFCQQGNTLPYTFG |
| with T2A and | GGTKLEITGGGGSGGGGSGGGGSEVKLQESGPGLVAPSQSLSVTCTVSGVSLPDYGVSWI |
| IL-15 | RQPPRKGLEWLGVIWGSETTYYNSALKSRLTIIKDNSKSQVFLKMNSLQTDDTAIYYCAK |
| | HYYYGGSYAMDYWGQGTSVTVSSEPKSCDKTHTCPPCPAPPVAGPSVFLFPPKPKDTLMI |
| | SRTPEVTCVVVDVSHEDPEVKFNWYVDGVEVHNAKTKPREEQYQSTYRVVSVLTVLHQDW |
| | LNGKEYKCKVSNKALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGNVFSCSVMHEALH |
| | NHYTQKSLSLSPGKIYIWAPLAGTCGVLLLSLVITLYCRSKRSRLLHSDYMNMTPRRPGP |
| | TRKHYQPYAPPRDFAAYRSERVQPLEENVGNAARPRFERNKRVKFSRSADAPAYQQGQNQ |
| | LYNELNLGRREEYDVLDKRRGRDPEMGGKPRRKNPQEGLYNELQKDKMAEAYSEIGMKGE |
| | RRRGKGHDGLYQGLSTATKDTYDALHMQALPPRGSGEGRGSLLTCGDVEENPGPMRISKP |
| | HLRSISIQCYLCLLLNSHFLTEAGIHVFILGCFSAGLPKTEANWVNVISDLKKIEDLIQS |
| | MHIDATLYTESDVHPSCKVTAMKCFLLELQVISLESGDASIHDTVENLIILANNSLSSNG |
| | NVTESGCKECEELEEKNIKEFLQSFVHIVQMFINTS- |
| SEQ ID NO: 57 | ATGGCACTTCCTGTTACAGCCCTCCTGCTCCCACTGGCTTTGCTGCTGCATGCTGCACGA |
| CD19 CAR-MU-8 | CCGGACATTCAAATGACACAAACTACGAGCTCCCTCTCCGCCTCCCTCGGTGACAGGGTG |
| with T2A and | ACCATCTCATGTCGGGCTAGTCAGGACATAAGCAAATACTTGAACTGGTACCAGCAAAAG |
| IL-15 | CCAGACGGGACCGTGAAGCTGCTCATCTATCACACTAGCCGCCTTCACTCTGGGGTCCCC |
| | TCTCGCTTTAGCGGTTCCGGCTCTGGTACAGACTATTCCCTTACTATTTCAAATCTTGAG |
| | CAGGAAGATATCGCTACATATTTTTGTCAACAGGGGAATACTCTTCCGTATACATTCGGG |
| | GGCGGAACAAAACTTGAAATTACAGGCGGCGGTGGTTCAGGAGGTGGTGGGAGCGGTGGG |
| | GGGGGATCAGAaGTtAAGCTGCAAGAATCAGGGCCCGGACTGGTAGCGCCCAGTCAAAGT |
| | CTTTCTGTTACTTGTACAGTATCCGGGGTGTCCCTTCCAGATTACGGGGTATCTTGGATT |
| | AGGCAACCTCCTCGGAAAGGATTGGAGTGGTTGGGTGTTATCTGGGGCAGTGAAACCACT |
| | TACTATAATAGTGCTCTTAAAAGTAGACTCACCATAATAAAGGATAATAGCAAGAGCCAG |
| | GTATTCCTGAAAATGAACAGCTTGCAAACGGATGATACCGCTATCTACTACTGCGCCAAA |
| | CACTATTATTATGGCGGTAGTTACGCTATGGATTATTGGGGCCAAGGaACGTCCGTCACC |
| | GTTAGTAGCGAGCCAAAGTCTTGCGACAAGACTCATACCTGCCCGCCTTGTCCCGCACCA |
| | CCTGTGGCAGGTCCCAGCGTGTTTCTGTTTCCGCCCAAGCCCAAGGACACACTCATGATC |
| | TCTAGGACACCCGAGGTTACATGCGTCGTGGTTGATGTTTCTCATGAAGACCCCGAAGTG |
| | AAGTTTAACTGGTATGTCGACGGAGTTGAGGTCCATAATGCTAAAACAAAACCACGCGAG |
| | GAACAATATCAAAGCACCTATCGGGTCGTTAGTGTCCTTACTGTACTTCACCAGGACTGG |
| | CTCAACGGAAAGGAATATAAGTGCAAAGTCAGCAATAAAGCCTTGCCAGCGCCCATCGAA |
| | AAAACTATCAGCAAGGCCAAGGGCCAACCCAGAGAACCCCAAGTGTACACACTCCCTCCT |
| | AGCCGGGATGAATTGACCAAAAATCAaGTCtctCTCACTTGTCTGGTAAAGGGATTCTAT |
| | CCAAGTGATATTGCTGTCGAATGGGAGAGCAACGGGCAACCGGAGAACAATTACAAAACG |
| | ACACCTCCCGTCCTTGATAGTGACGGGTCCTTCTTCCTGTATTCAAAGCTTACAGTCGAC |
| | AAAAGTCGCTGGCAGCAGGGGAATGTCTTTAGCTGTAGTGTCATGCACGAAGCTTTGCAT |
| | AACCACTACACGCAAAAGTCTCTTAGCCTGTCCCCAGGAAAGATCTACATTTGGGCCCCT |
| | CTGGCTGGAACATGTGGCGTGCTGCTGCTGTCCCTGGTCATTACTCTGTATTGTCGCTCA |
| | AAGCGCTCAAGATTGCTCCATTCTGATTACATGAACATGACCCCTCGCCGGCCGGGTCCC |
| | ACTCGCAAACACTACCAACCTTATGCCCCTCCGCGGGATTTCGCGGCCTACAGGTCAGAG |
| | AGAGTACAACCTCTTGAGGAGAATGTCGGCAACGCTGCCAGGCCGCGGTTTGAGCGAAAT |
| | AAGCGAGTCAAGTTTTCTCGAAGTGCCGACGCTCCTGCCTATCAGCAAGGCCAGAATCAA |
| | CTGTATAACGAGCTTAACTTGGGCCGGAGGGAGGAGTACGATGTGCTGGATAAGCGCAGG |
| | GGACGGGACCCGGAAATGGGCGGAAAGCCCAGGCGGAAAAACCCACAGGAAGGACTCTAC |
| | AATGAACTCCAAAAGGACAAAATGGCAGAGGCCTATTCAGAGATTGGGATGAAGGGAGAA |
| | AGGAGGCGAGGAAAAGGGCACGACGGCTTGTATCAGGGCTTGTCTACGGCCACTAAAGAC |
| | ACCTATGATGCCCTGCATATGCAGGCACTTCCCCCCAGAGGAAGTGGGGAAGGCCGAGGT |
| | TCATTGCTCACCTGTGGCGATGTGGAAGAAAATCCAGGTCCGATGCGCATAAGTAAGCCT |
| | CATCTGCGGTCCATTTCTATACAATGTTATCTGTGCTTGCTTTTGAACTCCCACTTTCTT |
| | ACGGAAGCAGGCATTCATGTGTTCATTCTGGGTTGTTTTTCtGCCGGGCTGCCCAAAACC |
| | GAGGCCAACTGGGTCAACGTGATCAGCGACCTCAAGAAGATCGAGGATTTGATTCAAAGT |
| | ATGCATATAGACGCCACACTCTATACTGAGTCCGACGTTCACCCGAGTTGTAAAGTTACG |
| | GCTATGAAGTGCTTTTTGTTGGAACTCCAGGTGATTTCCCTTGAATCCGGCGATGCGAGC |
| | ATCCACGATACGGTAGAGAATCTTATTATTCTGGCGAATAATTCTCTGTCTTCAAATGGG |
| | AATGTAACTGAGAGCGGTTGTAAAGAATGCGAAGAACTTGAAGAAAAGAATATCAAGGAA |
| | TTTCTTCAGAGTTTCGTGCATATTGTTCAAATGTTCATCAACACATCCTGA |
| SEQ ID NO: 58 | RASQDISKYL |
| anti-CD19 scFv | |
| CDRL1 | |
| SEQ ID NO: 59 | HTSRLHS |
| anti-CD19 scFv | |
| CDRL2 | |
| SEQ ID NO: 60 | QQGNTLPYT |
| anti-CD19 scFv | |
| CDRL3 | |
| SEQ ID NO: 61 | DYGVS |
| anti-CD19 scFv | |
| CDRH1 | |
| SEQ ID NO: 62 | VIWGSETTYYNSALKS |
| anti-CD19 scFv | |
| CDRH2 | |
| SEQ ID NO: 63 | HYYYGGSYAMDY |
| anti-CD19 scFv | |
| CDRH3 | |
| SEQ ID NO: 64 | DIQMTQTTSSLSASLGDRVTISCRASQDISKYLNWYQQKPDGTVKLLIYHTSRLHSGVPS |
| anti-CD19 scFv | RFSGSGSGTDYSLTISNLEQEDIATYFCQQGNTLPYTFGGGTKLEIT |
| VL | |
| SEQ ID NO: 65 | EVKLQESGPGLVAPSQSLSVTCTVSGVSLPDYGVSWIRQPPRKGLEWLGVIWGSETTYYN |
| anti-CD19 scFv | SALKSRLTIIKDNSKSQVFLKMNSLQTDDTAIYYCAKHYYYGGSYAMDYWGQGTSVTVSS |
| VH | |
| SEQ ID NO: 66 | MPPPRLLFFLLFLTPMEVRPEEPLVVKVEEGDNAVLQCLKGTSDGPTQQLTWSRESPLKP |
| CD19 | FLKLSLGLPGLGIHMRPLAIWLFIFNVSQQMGGFYLCQPGPPSEKAWQPGWTVNVEGSGE |
| | LFRWNVSDLGGLGCGLKNRSSEGPSSPSGKLMSPKLYVWAKDRPEIWEGEPPCLPPRDSL |
| | NQSLSQDLTMAPGSTLWLSCGVPPDSVSRGPLSWTHVHPKGPKSLLSLELKDDRPARDMW |
| | VMETGLLLPRATAQDAGKYYCHRGNLTMSFHLEITARPVLWHWLLRTGGWKVSAVTLAYL |
| | IFCLCSLVGILHLQRALVLRRKRKRMTDPTRRFFKVTPPPGSGPQNQYGNVLSLPTPTSG |
| | LGRAQRWAAGLGGTAPSYGNPSSDVQADGALGSRSPPGVGPEEEEGEGYEEPDSEEDSEF |
| | YENDSNLGQDQLSQDGSGYENPEDEPLGPEDEDSFSNAESYENEDEELTQPVARTMDFLS |
| | PHGSAWDPSREATSLGSQSYEDMRGILYAAPQLRSIRGQPGPNHEEDADSYENMDNPDGP |
| | DPAWGGGGRMGTWSTR |
| SEQ ID NO: 67 | GGGGSGGGGSGGGGS |
| SEQ ID NO: 68 | MKWVTFISLLFLFSSAYSRGVFRRDAHKSEVAHRFKDLGEENFKALVLIAFAQYLQQCPF |
| Human Albumin | EDHVKLVNEVTEFAKTCVADESAENCDKSLHTLFGDKLCTVATLRETYGEMADCCAKQEP |
| | ERNECFLQHKDDNPNLPRLVRPEVDVMCTAFHDNEETFLKKYLYEIARRHPYFYAPELLF |
| | FAKRYKAAFTECCQAADKAACLLPKLDELRDEGKASSAKQRLKCASLQKFGERAFKAWAV |
| | ARLSQRFPKAEFAEVSKLVTDLTKVHTECCHGDLLECADDRADLAKYICENQDSISSKLK |
| | ECCEKPLLEKSHCIAEVENDEMPADLPSLAADFVESKDVCKNYAEAKDVFLGMFLYEYAR |
| | RHPDYSVVLLLRLAKTYETTLEKCCAAADPHECYAKVFDEFKPLVEEPQNLIKQNCELFE |
| | QLGEYKFQNALLVRYTKKVPQVSTPTLVEVSRNLGKVGSKCCKHPEAKRMPCAEDYLSVV |
| | LNQLCVLHEKTPVSDRVTKCCTESLVNRRPCFSALEVDETYVPKEFNAETFTFHADICTL |
| | SEKERQIKKQTALVELVKHKPKATKEQLKAVMDDFAAFVEKCCKADDKETCFAEEGKKLV |
| | AASQAALGL |
| SEQ ID NO: 69 | DIQMTQTTSSLSASLGDRVTISCRASQDISKYLNWYQQKPDGTVKLLIYHTSRLHSGVPS |
| CD19 CAR-WT-28 | RFSGSGSGTDYSLTISNLEQEDIATYFCQQGNTLPYTFGGGTKLEITGGGGSGGGGSGGG |
| | GSEVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTSVTV |
| | SSEPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGKFWVLVV |
| | VGGVLACYSLLVTVAFIIFWVRSKRSRLLHSDYMNMTPRRPGPTRKHYQPYAPPRDFAAY |
| | RSERVQPLEENVGNAARPRFERNKRVKFSRSADAPAYQQGQNQLYNELNLGRREEYDVLD |
| | KRRGRDPEMGGKPRRKNPQEGLYNELQKDKMAEAYSEIGMKGERRRGKGHDGLYQGLSTA |
| | TKDTYDALHMQALPPR |
| SEQ ID NO: 70 | DIQMTQTTSSLSASLGDRVTISCRASQDISKYLNWYQQKPDGTVKLLIYHTSRLHSGVPS |
| CD19 CAR-MU-28 | RFSGSGSGTDYSLTISNLEQEDIATYFCQQGNTLPYTFGGGTKLEITGGGGSGGGGSGGG |
| | GSEVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTSVTV |
| | SSEPKSCDKTHTCPPCPAPPVAGPSVFLFPPKPKDTLMISRTPEVTCVVVDVSHEDPEVK |
| | FNWYVDGVEVHNAKTKPREEQYQSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKFWVLVVV |
| | GGVLACYSLLVTVAFIIFWVRSKRSRLLHSDYMNMTPRRPGPTRKHYQPYAPPRDFAAYR |
| | SERVQPLEENVGNAARPRFERNKRVKESRSADAPAYQQGQNQLYNELNLGRREEYDVLDK |
| | RRGRDPEMGGKPRRKNPQEGLYNELQKDKMAEAYSEIGMKGERRRGKGHDGLYQGLSTAT |
| | KDTYDALHMQALPPR |
| SEQ ID NO: 71 | DIQMTQTTSSLSASLGDRVTISCRASQDISKYLNWYQQKPDGTVKLLIYHTSRLHSGVPS |
| CD19 CAR-WT-8 | RFSGSGSGTDYSLTISNLEQEDIATYFCQQGNTLPYTFGGGTKLEITGGGGSGGGGSGGG |
| | GSEVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTSVTV |
| | SSEPKSCDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGKIYIWAP |
| | LAGTCGVLLLSLVITLYCRSKRSRLLHSDYMNMTPRRPGPTRKHYQPYAPPRDFAAYRSE |
| | RVQPLEENVGNAARPRFERNKRVKFSRSADAPAYQQGQNQLYNELNLGRREEYDVLDKRR |
| | GRDPEMGGKPRRKNPQEGLYNELQKDKMAEAYSEIGMKGERRRGKGHDGLYQGLSTATKD |
| | TYDALHMQALPPR |
| SEQ ID NO: 72 | DIQMTQTTSSLSASLGDRVTISCRASQDISKYLNWYQQKPDGTVKLLIYHTSRLHSGVPS |
| CD19 CAR-MU-8 | RFSGSGSGTDYSLTISNLEQEDIATYFCQQGNTLPYTFGGGTKLEITGGGGSGGGGSGGG |
| | GSEVKLQESGPGLVAPSQSLSVTCTVSGVSLPDYGVSWIRQPPRKGLEWLGVIWGSETTY |
| | YNSALKSRLTIIKDNSKSQVFLKMNSLQTDDTAIYYCAKHYYYGGSYAMDYWGQGTSVTV |
| | SSEPKSCDKTHTCPPCPAPPVAGPSVFLFPPKPKDTLMISRTPEVTCVVVDVSHEDPEVK |
| | FNWYVDGVEVHNAKTKPREEQYQSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKIYIWAPL |
| | AGTCGVLLLSLVITLYCRSKRSRLLHSDYMNMTPRRPGPTRKHYQPYAPPRDFAAYRSER |
| | VQPLEENVGNAARPRFERNKRVKFSRSADAPAYQQGQNQLYNELNLGRREEYDVLDKRRG |
| | RDPEMGGKPRRKNPQEGLYNELQKDKMAEAYSEIGMKGERRRGKGHDGLYQGLSTATKDT |
| | YDALHMQALPPR |
| SEQ ID NO: 73 | DIQMTQTTSSLSASLGDRVTISCRASQDISKYLNWYQQKPDGTVKLLIYHTSRLHSGVPS |
| Anti-CD19 CAR | RFSGSGSGTDYSLTISNLEQEDIATYFCQQGNTLPYTFGGGTKLEITGGGGSGGGGSGGG |
| (CD28-CD3z- | GSEVKLQESGPGLVAPSQSLSVTCTVSGVSLPDYGVSWIRQPPRKGLEWLGVIWGSETTY |
| IL15) | YNSALKSRLTIIKDNSKSQVFLKMNSLQTDDTAIYYCAKHYYYGGSYAMDYWGQGTSVTV |
| | SSEPKSCDKTHTCPPCPAPPVAGPSVFLFPPKPKDTLMISRTPEVTCVVVDVSHEDPEVK |
| | FNWYVDGVEVHNAKTKPREEQYQSTYRVVSVLTVLHQDWLNGKEYKCKVSNKALPAPIEK |
| | TISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGKFWVLVVV |
| | GGVLACYSLLVTVAFIIFWVRSKRSRLLHSDYMNMTPRRPGPTRKHYQPYAPPRDFAAYR |
| | SRVKFSRSADAPAYQQGQNQLYNELNLGRREEYDVLDKRRGRDPEMGGKPRRKNPQEGLY |
| | NELQKDKMAEAYSEIGMKGERRRGKGHDGLYQGLSTATKDTYDALHMQALPPR |
| SEQ ID NO: 74 | GACATTCAAATGACACAAACTACGAGCTCCCTCTCCGCCTCCCTCGGTGACAGGGTGACC |
| Anti-CD19 CAR | ATCTCATGTCGGGCTAGTCAGGACATAAGCAAATACTTGAACTGGTACCAGCAAAAGCCA |
| (CD28-CD3z- | GACGGGACCGTGAAGCTGCTCATCTATCACACTAGCCGCCTTCACTCTGGGGTCCCCTCT |
| IL15) | CGCTTTAGCGGTTCCGGCTCTGGTACAGACTATTCCCTTACTATTTCAAATCTTGAGCAG |
| | GAAGATATCGCTACATATTTTTGTCAACAGGGGAATACTCTTCCGTATACATTCGGGGGC |
| | GGAACAAAACTTGAAATTACAGGCGGCGGTGGTTCAGGAGGTGGTGGGAGCGGTGGGGGG |
| | GGATCAGAaGTtAAGCTGCAAGAATCAGGGCCCGGACTGGTAGCGCCCAGTCAAAGTCTT |
| | TCTGTTACTTGTACAGTATCCGGGGTGTCCCTTCCAGATTACGGGGTATCTTGGATTAGG |
| | CAACCTCCTCGGAAAGGATTGGAGTGGTTGGGTGTTATCTGGGGCAGTGAAACCACTTAC |
| | TATAATAGTGCTCTTAAAAGTAGACTCACCATAATAAAGGATAATAGCAAGAGCCAGGTA |
| | TTCCTGAAAATGAACAGCTTGCAAACGGATGATACCGCTATCTACTACTGCGCCAAACAC |
| | TATTATTATGGCGGTAGTTACGCTATGGATTATTGGGGCCAAGGaACGTCCGTCACCGTT |
| | AGTAGCGAGCCAAAGTCTTGCGACAAGACTCATACCTGCCCGCCTTGTCCCGCACCACCT |
| | GTGGCAGGTCCCAGCGTGTTTCTGTTTCCGCCCAAGCCCAAGGACACACTCATGATCTCT |
| | AGGACACCCGAGGTTACATGCGTCGTGGTTGATGTTTCTCATGAAGACCCCGAAGTGAAG |
| | TTTAACTGGTATGTCGACGGAGTTGAGGTCCATAATGCTAAAACAAAACCACGCGAGGAA |
| | CAATATCAAAGCACCTATCGGGTCGTTAGTGTCCTTACTGTACTTCACCAGGACTGGCTC |
| | AACGGAAAGGAATATAAGTGCAAAGTCAGCAATAAAGCCTTGCCAGCGCCCATCGAAAAA |
| | ACTATCAGCAAGGCCAAGGGCCAACCCAGAGAACCCCAAGTGTACACACTCCCTCCTAGC |
| | CGGGATGAATTGACCAAAAATCAaGTCtctCTCACTTGTCTGGTAAAGGGATTCTATCCA |
| | AGTGATATTGCTGTCGAATGGGAGAGCAACGGGCAACCGGAGAACAATTACAAAACGACA |
| | CCTCCCGTCCTTGATAGTGACGGGTCCTTCTTCCTGTATTCAAAGCTTACAGTCGACAAA |
| | AGTCGCTGGCAGCAGGGGAATGTCTTTAGCTGTAGTGTCATGCACGAAGCTTTGCATAAC |
| | CACTACACGCAAAAGTCTCTTAGCCTGTCCCCAGGAAAGTTCTGGGTACTGGTGGTTGTG |
| | GGCGGCGTTCTGGCTTGCTATTCACTCCTCGTTACGGTTGCATTTATAATATTTTGGGTA |
| | CGCTCAAAGCGCTCAAGATTGCTCCATTCTGATTACATGAACATGACCCCTCGCCGGCCG |
| | GGTCCCACTCGCAAACACTACCAACCTTATGCCCCTCCGCGGGATTTCGCGGCCTACAGG |
| | TCACGAGTCAAGTTTTCTCGAAGTGCCGACGCTCCTGCCTATCAGCAAGGCCAGAATCAA |
| | CTGTATAACGAGCTTAACTTGGGCCGGAGGGAGGAGTACGATGTGCTGGATAAGCGCAGG |
| | GGACGGGACCCGGAAATGGGCGGAAAGCCCAGGCGGAAAAACCCACAGGAAGGACTCTAC |
| | AATGAACTCCAAAAGGACAAAATGGCAGAGGCCTATTCAGAGATTGGGATGAAGGGAGAA |
| | AGGAGGCGAGGAAAAGGGCACGACGGCTTGTATCAGGGCTTGTCTACGGCCACTAAAGAC |
| | ACCTATGATGCCCTGCATATGCAGGCACTTCCCCCCAGA |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-CD19 chimeric antigen receptor (CAR) comprising:
an extracellular antigen binding domain comprising an anti-CD19 antibody or antigen binding fragment thereof, and
an intracellular signaling region comprising an OX40L intracellular signaling domain.

2. The anti-CD19 CAR of claim 1, wherein the OX40L intracellular signaling domain comprises an amino acid sequence set forth in SEQ ID NO: 8, SEQ ID NO: 9, or SEQ ID NO: 10.

3. The anti-CD19 CAR of claim 1, wherein the OX40L intracellular signaling domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 8, SEQ ID NO: 9, or SEQ ID NO: 10.

4. The anti-CD19 CAR of claim 1, wherein the anti-CD19 antibody or antigen binding fragment thereof comprises a light chain complementarity determining region 1 (CDRL1) comprising SEQ ID NO: 58, a light chain complementarity determining region 2 (CDRL2) comprising SEQ ID NO: 59; a light chain complementarity determining region 3 (CDRL3) comprising SEQ ID NO: 60, a heavy chain complementarity determining region 1 (CDRH1 comprising SEQ ID NO: 61; a heavy chain complementarity determining region 2 (CDRH2) comprising SEQ ID NO: 62; and a heavy chain complementarity determining region 3 (CDRH3) comprising SEQ ID NO: 63.

5. The anti-CD19 CAR of claim 1, wherein the anti-CD19 antibody or antigen binding fragment thereof comprises a light chain variable (V_{L}) region comprising SEQ ID NO: 64 and a heavy chain variable (V_{H}) region comprising SEQ ID NO: 65.

6. The anti-CD19 CAR of claim 1, wherein the anti-CD19 antibody or antigen binding fragment thereof comprises a V_{L} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 64 and a V_{H} region comprising an amino acid sequence having or having at least 85%, 86%, 87%, 88%, 89%, 90%, 91%, 92%, 93%, 94%, 95%, 96%, 97%, 98%, or 99% identity to SEQ ID NO: 65.

7.-12. (canceled)

13. The anti-CD19 CAR of claim 1, wherein the extracellular antigen binding domain comprises the amino acid sequence set forth in SEQ ID NO: 30.

14. The anti-CD19 CAR of claim 1, wherein the extracellular antigen binding domain comprises an amino acid sequence having or having at least 85%, 86%, 87%, 88%, 89%, 90%, 91%, 92%, 93%, 94%, 95%, 96%, 97%, 98%, or 99% identity to SEQ ID NO: 30.

15. The anti-CD19 CAR of claim 1, wherein the anti-CD19 CAR specifically binds to a B-lymphocyte antigen CD19 (CD19) protein.

16. The anti-CD19 CAR of claim 15, wherein the CD19 protein comprises the amino acid sequence of SEQ ID NO: 66.

17. The anti-CD19 CAR of claim 1, wherein the CAR comprises transmembrane region, optionally a CD28 transmembrane domain.

18. (canceled)

19. The anti-CD19 CAR of claim 17, further comprising a hinge domain between the extracellular antigen binding domain and the transmembrane domain.

20.-22. (canceled)

23. The anti-CD19 CAR of claim 1, wherein the intracellular signaling region further comprises a CD28 intracellular signaling domain.

24. The anti-CD19 CAR of claim 1, wherein the intracellular signaling region further comprises a CD3-zeta (CD3ζ) signaling domain.

25.-33. (canceled)

34. A polynucleotide comprising a nucleic acid encoding the anti-CD19 CAR of claim 1.

35.-52. (canceled)

53. A vector or a cell comprising the polynucleotide of claim 34.

54.-63. (canceled)

64. A population of cells comprising a plurality of the cells of claim 53.

65. (canceled)

66. (canceled)

67. A pharmaceutical composition comprising the population of cells of claim 64.

68.-83. (canceled)

84. A frozen vial comprising the composition of claim 67.

85. A method of treatment comprising administering the composition of claim 67 to a subject having a disease or condition associated with CD19.

86.-129. (canceled)
